(19) United States
(12) Patent Application Publication
(10) Pub. No.: US 2003/0185848 A1
(43) Pub. Date: Oct. 2, 2003

(54) Methods and compositions for vaccination comprising nucleic acid and/or polypeptide sequences of Chlamydia psittaci

(75) Inventors: Stephen A. Johnston, Dallas, TX (US); Katherine Stemke-Hale, Dallas, TX (US); Kathryn F. Sykes, Dallas, TX (US); Bernhard Kaltenboeck, Auburn, AL (US)

Correspondence Address: FULBRIGHT & JAWORSKI L.L.P., SUITE 2400, 600 CONGRESS AVENUE, AUSTIN, TX, 78701, US

(73) Assignee: Board of Regents, The University of Texas System

(21) Appl. No.: 09/738,269

(22) Filed: Dec. 15, 2000

(51) Int. Cl. A61K 39/02; A61K 48/00; C07H 21/04; C12N 1/21; C12N 15/74; C07K 14/295

(52) U.S. Cl. 424/190.1; 514/44; 435/69.3; 435/252.3; 435/320.1; 530/350; 536/23.7

(57) ABSTRACT

The instant invention relates to antigens and nucleic acids encoding such antigens obtainable by screening the Chlamydia psittaci genome. In more specific aspects, the invention relates to methods of isolating such antigens and nucleic acids and to methods of using such isolated antigens for producing immune responses in bovines or other non-human animals. The ability of an antigen to produce an immune response may be employed in vaccination of bovines or antibody preparation techniques.

The government owns rights in the present invention pursuant to DARPA grant number MDA 972-97-1-0013.

### BACKGROUND OF THE INVENTION

1. Field of the Invention

The present invention relates generally to the fields of immunology, bacteriology and molecular biology. More particularly, the invention relates to methods for screening and obtaining vaccines generated from the administration of expression libraries constructed from a Chlamydia psittaci genome. In particular embodiments, it concerns methods and compositions for the vaccination of vertebrate animals against Chlamydia psittaci bacterial infections, wherein vaccination of the animal is via a protein or gene derived from part or all of the genes validated as vaccines.

2. Description of Related Art

Intracellular bacteria of the genus Chlamydia are important pathogens in both man and vertebrate animals, causing blindness in man, sexually transmitted disease, and community-acquired pneumonia, and most likely act as co-factors in atherosclerotic plaque formation in human coronary heart disease.

Ubiquitous Chlamydia (C) psittaci infections in cattle cause mastitis, infertility and abortion. A primary economic impact of Chlamydia psittaci in dairy cattle is the loss of milk production and quality. Serological evidence for infection with ruminant C. psittaci is found in virtually all cattle (Kaltenbock et al., 1997). These infections typically do not cause overt signs of disease, but under stress of the host animal may elicit transient inflammation of the mammary gland and uterus. These stress-related herd health problems, while not clinically pronounced, result in major losses for animal agriculture due to reduced output and quality of animal products like milk.

Most existing vaccines for the treatment of bacterial infections are composed of live/attenuated or killed pathogens (Babiuk, 1999). Live/attenuated vaccines present the risk of residual, or reacquisition of, pathogenicity, and are associated with a high cost of production. In addition, efficacious live/attenuated vaccines cannot be developed against many pathogens, or are impractical to produce. Killed pathogens typically have less utility than live/attenuated vaccines, as they are not usually effective in eliciting cellular immune responses. An alternative is subunit vaccines that consist of one or a few proteins of the pathogen (Babiuk, 1999; Ellis, 1999). The proteins being developed for these vaccines are typically based on a dominant immune response in infected hosts, and/or on surmised importance in the disease process. Due to the high genetic complexity of bacteria or protozoa, the empirical approach to identify these proteins often requires extensive research on the pathogen's biology and produces a small, biased set of potential vaccine candidates. However, this is currently the only practical method when proteins are the commodity for testing a vaccine.

The development of genetic (DNA) immunization (Tang et al., 1992) not only offers a new method of vaccine delivery, but also enables a new, unbiased, approach to vaccine discovery. One of the inventors proposed that the whole genome of a pathogen could be searched for protein vaccine candidates by directly assessing protection from challenge, termed expression library immunization (ELI) (U.S. Pat. No. 5,703,057, specifically incorporated herein by reference). It involves making an expression library representing the whole genome of the pathogen in a genetic immunization vector. The library is subdivided into smaller groups, and DNA from each library is used to vaccinate animals that are subsequently challenged. The advantage of this approach is that all of the potentially protective genes could be discovered and used in any useful combination to reconstitute a vaccine devoid of non-protective, immunopathological, or immunosuppressive antigens. The potential of ELI was demonstrated in a murine Mycoplasma pulmonis infection, against which random M. pulmonis libraries were protective (Barry et al., 1995). Since then, others have reported on protective libraries (Brayton et al., 1998; Piedrafita et al., 1999), but the reduction of these libraries to individual genes has not been demonstrated.

As described above, the widespread human and animal infections by the genus Chlamydia psittaci represents a particular challenge for vaccinology. C. psittaci infections in cattle cause mastitis, infertility and abortion. A primary economic impact of Chlamydia psittaci in dairy cattle is the loss of milk production and quality. Thus, an effective vaccine against Chlamydia psittaci bacterial infections in cattle would be of great economic importance. However, there presently have been no effective vaccines developed against Chlamydia psittaci.

### SUMMARY OF THE INVENTION

In some embodiments, the invention relates to isolated polynucleotides having a region that comprises a sequence of SEQ ID NO:6, SEQ ID NO:8, SEQ ID NO:10, SEQ ID NO:12, SEQ ID NO:14, SEQ ID NO:16, SEQ ID NO:18, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:58, or SEQ ID NO:60, a complement of any of these sequences or fragments thereof. In some more specific embodiments, the invention relates to such polynucleotide comprising a region having a sequence comprising at least 17, 20, 25, 30, 35, 40, 45, 50, 60, 70, 80, 90, 100, 125, 150, 200, or more contiguous nucleotides in common with at least one of SEQ ID NO:6, SEQ ID NO:8, SEQ ID NO:10, SEQ ID NO:12, SEQ ID NO:14, SEQ ID NO:16, SEQ ID NO:18, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:58, or SEQ ID NO:60, or its complement. Of course, such polynucleotides may comprise a region having all nucleotides in common with at least one of SEQ ID NO:6, SEQ ID NO:8, SEQ ID NO:δ0, SEQ ID NO:12, SEQ ID NO:14, SEQ ID NO:16, SEQ ID NO: 18, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:58, or SEQ ID NO:60, or its complement.

In other aspect, the invention relates to polypeptides having sequences of SEQ ID NO:7, SEQ ID NO:9, SEQ ID NO:1, SEQ ID NO:13, SEQ ID NO:15, SEQ ID NO:17, SEQ ID NO:19, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:59, SEQ ID NO:61 or fragments thereof. The invention also relates to methods of producing such polypeptides using recombinant methods, for example, using the polynucleotides described above.

The invention relates to antibodies against Chlamydia psittaci antigens, including those directed against an antigen having sequences of SEQ ID NO:7, SEQ ID NO:9, SEQ ID NO:1, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, or SEQ ID NO:61, or an antigenic fragment thereof. The antibodies may be polyclonal or monoclonal and produced by methods known in the art.

The invention relates to vaccines for the immunization of bovines against Chlamydia psittaci. Such vaccines may comprise a pharmaceutically acceptable carrier, and at least one polynucleotide having a Chlamydia psittaci sequence. Such vaccines may comprise at least one polynucleotide that has a sequence isolated from a Chlamydia psittaci genomic DNA expression library. In some preferred embodiments, the vaccine comprises at least one polynucleotide having a sequence of SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO52:, SEQ ID NO:54, SEQ ID NO:56, SEQ ID NO: 58, or SEQ ID NO:60, or fragment thereof. In some specific preferred embodiments, the vaccine comprises at least one polynucleotide having a sequence of SEQ ID NO:6, SEQ ID NO:8, SEQ ID NO:10, SEQ ID NO:12, SEQ ID NO:14, SEQ ID NO:16, SEQ ID NO:20, SEQ ID NO:22, SEQ ID NO:24, or SEQ ID NO:26, or fragment thereof, while in some even more specific embodiments, the vaccine comprises at least one polynucleotide having a sequence of SEQ ID NO:6, SEQ ID NO:10, SEQ ID NO:14, SEQ ID NO:20, or SEQ ID NO:24. The polynucleotide may be comprised in a genetic immunization vector. Some vectors useful in the invention comprise a gene encoding a mouse ubiquitin fusion polypeptide and/or a promoter operable in eukaryotic cells, for example a CMV promoter. The polynucleotide may be cloned into a viral expression vector, for example, a viral expression vector selected from the group consisting of adenovirus, adeno-associated virus, retrovirus and herpes-simplex virus.

In some embodiments, the vaccine comprises at least a first polynucleotide having a Chlamydia psittaci sequence and a second polynucleotide having a Chlamydia psittaci sequence, wherein the first polynucleotide and the second polynucleotide have different Chlamydia psittaci sequences. In some preferred embodiments, the first polynucleotide has a sequence of SEQ ID NO:50.

Other embodiments of vaccines for the immunization of a bovine against Chlamydia psittaci comprise a pharmaceutically acceptable carrier and at least one Chlamydia psittaci antigen. The at least one Chlamydia psittaci antigen can be an antigen having a sequence of SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, or SEQ ID NO:61, or an antigenic fragment thereof. In some specific embodiments, the vaccine comprises at least one Chlamydia psittaci antigen having a sequence of SEQ ID NO:7, SEQ ID NO:9, SEQ ID NO:11, SEQ ID NO:13, SEQ ID NO:15, SEQ ID NO:17, SEQ ID NO:21, SEQ ID NO:23, SEQ ID NO:25, or SEQ ID NO:27 or an antigenic fragment thereof. In some even more specific embodiments, the at least one Chlamydia psittaci antigen has a sequence of SEQ ID NO:7, SEQ ID NO:11, SEQ ID NO:15, SEQ ID NO:21, or SEQ ID NO:25.

The invention contemplates methods of immunizing a bovine comprising providing to the bovine at least one Chlamydia psittaci antigen, or antigenic fragment thereof, in an amount effective to induce an immune response. The antigens described above are examples of particularly useful antigens in this regard. The provision of at least one Chlamydia psittaci antigen may comprise: (a) preparing a cloned expression library from fragmented genomic DNA, cDNA or sequenced genes of Chlamydia psittaci; (b) administering at least one clone of the library in a pharmaceutically acceptable carrier into the bovine; and (c) expressing at least one Chlamydia psittaci antigen in the bovine. The polynucleotide may be administered, for example, by a intramuscular injection or epidermal injection or intravenous, subcutaneous, intralesional, intraperitoneal, oral or inhaled routes of administration. An intramuscular injection may comprise least 1.0 μg to 200 μg of the polynucleotide, whereas an epidermal injection may comprise at least 0.01 μg to 5.0 μg of the polynucleotide. Second intramuscular injection or epidermal injections may be administered, for example, at least about three weeks after the first injection. The polynucleotide may be comprised in a viral expression vector.

Alternatively, the provision of the Chlamydia antigen(s) may comprise: (a) preparing a pharmaceutical composition comprising at least one polynucleotide having a sequence of SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO52:, SEQ ID NO:54, SEQ ID NO:56, SEQ ID NO: 58, or SEQ ID NO:60, or fragment thereof; (b) administering one or more clones of the library in a pharmaceutically acceptable carrier into the bovine; and (c) expressing one or more Chlamydia antigens in the bovine. The antigen may be administered in much the same manner as described for polynucleotides above, and other manners known to those of skill in the art.

In another alternative, the provision of the Chlamydia antigen(s) may comprise: (a) preparing a pharmaceutical composition of at least one Chlamydia antigen having a sequence of SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, or SEQ ID NO:61, or an antigenic fragment thereof; and (b) administering the at least one antigen or fragment into the animal.

This specification discloses methods of obtaining polynucleotide sequences effective for generating an immune response against Chlamydia psittaci comprising: (a) preparing a cloned expression library from fragmented genomic DNA of the species Chlamydia psittaci; (b) administering one or more clones of the library in a pharmaceutically acceptable carrier into the animal in an amount effective to induce an immune response; and (c) selecting from the library the polynucleotide sequences that induce an immune response, wherein the immune response in the animal is protective against Chlamydia psittaci infection. Such methods may further comprise testing the animal for immune resistance against a Chlamydia psittaci bacterial infection by challenging the animal with Chlamydia psittaci. The genomic DNA is fragmented physically or by restriction enzymes, and in some preferred embodiments, the fragments are about 200-1000 base pairs. In some cases each clone in the library may comprise a gene encoding a mouse ubiquitin fusion polypeptide designed to link the expression library polynucleotides to the ubiquitin gene. In some embodiments, the library is about 1×10³ to about 1×10⁶ clones, with some preferred embodiments using a library having 1×10⁵ clones. In some cases, about 0.01 μg to about 200 μg of DNA, cDNA or sequenced gene from the clones is administered into the animal, for example by intramuscular injection or epidermal injection. In many cases, the cloned expression library further comprises a promoter operably linked to the DNA that permits expression in a vertebrate animal cell.

The invention also relates to methods of assaying for the presence of Chlamydia psittaci infection in a bovine comprising: (a) obtaining an antibody directed against a Chlamydia psittaci antigen; (b) obtaining a sample from the bovine; (c) admixing the antibody with the sample; and (d) assaying the sample for antigen-antibody binding, wherein the antigen-antibody binding indicates Chlamydia psittaci infection in the bovine. In many cases, the antibody directed against the antigen is a monoclonal antibody. In some preferred embodiments, assaying the sample for antigen-antibody binding is done by precipitin reaction, radioimmunoassay, ELISA, Western blot or immunofluorescence. The invention also relates to kits for assaying bovines for a Chlamydia psittaci infection comprising, in a suitable container: (a) a pharmaceutically acceptable carrier; and (b) an antibody directed against a Chlamydia psittaci antigen. The method also relates to a method of assaying for the presence of a Chlamydia psittaci infection in a bovine comprising: (a) obtaining an oligonucleotide probe comprising a sequence comprised within one of SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO52:, SEQ ID NO:54, SEQ ID NO:56, SEQ ID NO: 58, or SEQ ID NO:60, or a complement thereof; and (b) employing the probe in a PCR detection protocol. Kits for such protocols are also within the scope of the invention.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Scheme for Expression Library Immunization.

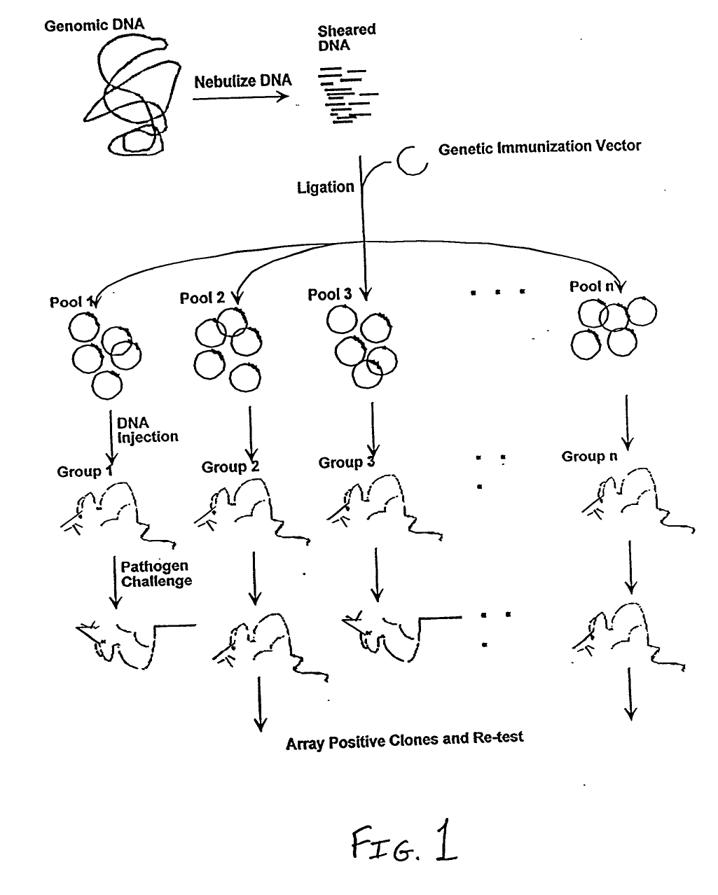

FIG. 2. Production of the C. psittaci Library. The C. psittaci library was produced by first physically shearing the genomic DNA, strain BGM/B577, and size selecting fragments of 300-800 base pairs. The fragments were ligated into the Bgl II site of pCMVi-Ubs(+P3); see Sykes and Johnston, 1999 for details. The nucleotide sequence shown in this figure is given as SEQ ID NO:1.

FIG. 3. Flowchart depicting the process for deconvolution of the libraries. Each round consists of preparation of DNA samples, vaccination of mice, challenge and determination of the relative protection in each group.

FIG. 4. Results of protection assays in Rounds 1, 2 and 3. Protection was scored as lung weight relative to average of the vaccinated, maximum protection, positive control and the non-vaccinated, challenged, maximum disease, negative control. The relative protection score was calculated by assigning the score 1 to animals with lung weight equal to the vaccinated control and the score 0 to animals with lung weights equal to the challenged, non-vaccinated control. These points define a line; animals with lower lung weight, hence better protection, have a higher relative protection score. Animals that have worse disease than challenged, non-vaccinated controls, i.e. heavier lungs, will have a negative relative protection score. The unchallenged Naive group consistently had lung weights slightly lower than the maximum protection, positive controls (Vaccinated) due to the peribronchiolar accumulation of lymphatic cells. In Rounds 2 and 3 the pools of plasmids from columns in the two-dimensional arrays are assigned numbers and the rows assigned letters. The solid bars indicate pools that were designated as protective and entered into the subsequent round. The error bars represent one standard deviation on either side of the mean.

FIG. 5. Results of protection assays of testing individual gene fragments in Round 4. Protection was scored as lung weight relative to the average of the vaccinated, maximum protection, positive control (Vaccinated=1) and the non-vaccinated, challenged, maximum disease, negative control (Challenged=0). The Pool<50AA is the DNA consisting of the pool of the 32 plasmids from Round 3 having predicted open-reading frames less than 50 amino acids long. Pool>50AA is the DNA consisting of all the 14 plasmids containing C. psittaci inserts encoding in-frame proteins more than 50 amino acids long. The numbers of each individual gene fragment tested correspond to the numbers in FIG. 4. The error bars represent one standard deviation of the mean.

FIG. 6. Summary of characterization of the single gene fragments of Round 4. The Relative Protection score of each C. psittaci (CP) gene fragment is provided along with the designation of the gene in C. pneumonia that has the highest similarity (C. pneumonia homolog). In two cases, gene fragment CP #4 and CP #12, the C. psittaci gene could also be identified. On the right is a linear map showing the location in each gene of the fragment that conferred protection (shaded).

FIG. 7. Protection data from DNA pools. CP1-6 is a negative pool from round 1. To test whether a single protective gene could be detected in a negative pool, 25 ng of either CP4 #4 or CP4 #11 was added to 50 μg of CP1-6.

### DESCRIPTION OF ILLUSTRATIVE EMBODIMENTS

The present invention provides compositions and methods for the immunization of vertebrate animals, including humans, against infections using nucleic acid sequences and polypeptides elucidated by screening Chlamydia psittaci. These compositions and methods will be useful for immunization against Chlamydia psittaci bacterial infections and other infections and disease states. In particular embodiments, a vaccine composition directed against Chlamydia psittaci infections is provided. The vaccine according to the present invention comprises Chlamydia psittaci genes and polynucleotides identified by the inventors, that confer protective resistance in vertebrate animals to Chlamydia psittaci bacterial infections, and other infections. In other embodiments, the invention provides methods for immunizing an animal against Chlamydia psittaci infections, methods for preparing a cloned library via expression library immunization and methods for screening and identifying Chlamydia psittaci genes that confer protection against infection.

A. Expression Library Immunization

In particular embodiments, the immunization of vertebrate animals according to the present invention includes a cloned library of Chlamydia psittaci expression constructs. In specific embodiments, a cloned expression library of Chlamydia psittaci is provided. Expression library immunization, ELI herein, is well known in the art (U.S. Pat. No. 5,703,057, specifically incorporated herein by reference). In certain embodiments, the invention provides an ELI method applicable to virtually any pathogen and requires no knowledge of the biological properties of the pathogen. The method operates on the assumption, generally accepted by those skilled in the art, that all the potential antigenic determinants of any pathogen are encoded in its genome. The inventors have previously devised methods of identifying vaccines using a genomic expression library representing all of the antigenic determinants of a pathogen (U.S. Pat. No. 5,703,057). The method uses to its advantage the simplicity of genetic immunization to sort through a genome for immunological reagents in an unbiased, systematic fashion.

The preparation of an expression library is performed using the techniques and methods familiar one of skill in the art. The pathogen's genome, may or may not be known or possibly may even have been cloned. Thus one obtains DNA (or cDNA), representing substantially the entire genome of the pathogen (e.g., Chlamydia psittaci). The DNA is broken up, by physical fragmentation or restriction endonuclease, into segments of some length so as to provide a library of about 10⁵ (approximately 18× the genome size) members. The library is then tested by inoculating a subject with purified DNA of the library or sub-library and the subject challenged with a pathogen, wherein immune protection of the subject from pathogen challenge indicates a clone that confers a protective immune response against infection.

B. Nucleic Acids

The present invention provides Chlamydia psittaci polynucleotide compositions and methods that induce a protective immune response in vertebrate animals challenged with a Chlamydia psittaci bacterial infection. The preparation and purification of antigenic Chlamydia psittaci polypeptides, or fragments thereof (Section C) and antibody preparations directed against Chlamydia psittaci antigens, or fragments thereof (Section E) are described below.

Thus, in certain embodiments of the present invention, genes or polynucleotides encoding Chlamydia psittaci polypeptides or fragments thereof are provided. It is contemplated in other embodiments, that a polynucleotide encoding a Chlamydia psittaci polypeptide or polypeptide fragment will be expressed in prokaryotic or eukaryotic cells and the polypeptides purified for use as anti-Chlamydia psittaci antigens in the vaccination of vertebrate animals or in generating antibodies immunoreactive with Chlamydia psittaci polypeptides (i.e., antigens). The genomes of C. pneumoniae and C. trachomatis have been completely sequenced. The Chlamydia genes are quite similar, with the four most protective genes identified being 30-71% identical and 45-85% similar in amino acid sequence.

1. Nucleic Acids Encoding Chlamydia psittaci Polypeptides

The present invention provides polynucleotides encoding antigenic Chlamydia psittaci polypeptides capable of inducing a protective immune response in vertebrate animals and for use as an antigen to generate anti-Chlamydia psittaci or other pathogen antibodies. In certain instances, it may be desirable to express Chlamydia psittaci polynucleotides encoding a particular antigenic Chlamydia psittaci polypeptide domain or sequence to be used as a vaccine or in generating anti-Chlamydia psittaci or other pathogen antibodies. Nucleic acids according to the present invention may encode an entire Chlamydia psittaci gene, or any other fragment of the Chlamydia psittaci sequences set forth herein. The nucleic acid may be derived from genomic DNA, i.e., cloned directly from the genome of a particular organism. In other embodiments, however, the nucleic acid may comprise complementary DNA (cDNA). A protein may be derived from the designated sequences for use in a vaccine or to isolate useful antibodies.

The term “cDNA” is intended to refer to DNA prepared using messenger RNA (mRNA) as template. The advantage of using a cDNA, as opposed to genomic DNA or DNA polymerized from a genomic, non- or partially-processed RNA template, is that the cDNA primarily contains coding sequences of the corresponding protein. There may be times when the full or partial genomic sequence is preferred, such as where the non-coding regions are required for optimal expression.

It also is contemplated that a given Chlamydia psittaci polynucleotide may be represented by natural variants that have slightly different nucleic acid sequences but, nonetheless, encode the same polypeptide (see Table 2 below). In addition, it is contemplated that a given Chlamydia psittaci polypeptide may be generated using alternate codons that result in a different nucleic acid sequence but encodes the same polypeptide.

As used in this application, the term “a nucleic acid encoding a Chlamydia psittaci polynucleotide” refers to a nucleic acid molecule that has been isolated free of total cellular nucleic acid. The term “functionally equivalent codon” is used herein to refer to codons that encode the same amino acid, such as the six codons for arginine or serine (Table 2, below), and also refers to codons that encode biologically equivalent amino acids, as discussed in the following pages.

TABLE 1

| Amino Acids | | | Codons | | | | | |
|---|---|---|---|---|---|---|---|---|
| Alanine | Ala | A | GCA | GCC | GCG | GCU | | |
| Cysteine | Cys | C | UGC | UGU | | | | |
| Aspartic acid | Asp | D | GAC | GAU | | | | |
| Glutamic acid | Glu | E | GAA | GAG | | | | |
| Phenylalanine | Phe | F | UUC | UUU | | | | |
| Glycine | Gly | G | GGA | GGC | GGG | GGU | | |
| Histidine | His | H | CAC | CAU | | | | |
| Isoleucine | Ile | I | AUA | AUC | AUU | | | |
| Lysine | Lys | K | AAA | AAG | | | | |
| Leucine | Leu | L | UUA | UUG | CUA | CUC | CUG | CUU |
| Methionine | Met | M | AUG | | | | | |
| Asparagine | Asn | N | AAC | AAU | | | | |
| Proline | Pro | P | CCA | CCC | CCG | CCU | | |
| Glutamine | Gln | Q | CAA | CAG | | | | |
| Arginine | Arg | R | AGA | AGG | CGA | CGC | CGG | CGU |
| Serine | Ser | S | AGC | AGU | UCA | UCC | UCG | UCU |
| Threonine | Thr | T | ACA | ACC | ACG | ACU | | |
| Valine | Val | V | GUA | GUC | GUG | GUU | | |
| Tryptophan | Trp | W | UGG | | | | | |
| Tyrosine | Tyr | Y | UAC | UAU | | | | |

Allowing for the degeneracy of the genetic code, sequences that have at least about 50%, usually at least about 60%, more usually about 70%, most usually about 80%, preferably at least about 90% and most preferably about 95% of nucleotides that are identical to the nucleotides of given Chlamydia psittaci gene or polynucleotide. Sequences that are essentially the same as those set forth in a Chlamydia psittaci gene or polynucleotide may also be functionally defined as sequences that are capable of hybridizing to a nucleic acid segment containing the complement of a Chlamydia psittaci polynucleotide under standard conditions.

The DNA segments of the present invention include those encoding functional and/or immunologically equivalent Chlamydia psittaci proteins and peptides, as described above. Such sequences may arise as a consequence of codon redundancy and amino acid functional equivalency that are known to occur naturally within nucleic acid sequences and the proteins thus encoded. Alternatively, functionally and/or immunogenically equivalent proteins or peptides may be created via the application of recombinant DNA technology, in which changes in the protein structure may be engineered, based on considerations of the properties of the amino acids being exchanged. Changes designed by man may be introduced through the application of site-directed mutagenesis techniques or may be introduced randomly and screened later for the desired function, as described below.

2. Oligonucleotide Sequences

Naturally, the present invention also encompasses DNA segments that are complementary, or essentially complementary to the sequences of a Chlamydia psittaci polynucleotide. Nucleic acid sequences that are “complementary” are those that are capable of base-pairing according to the standard Watson-Crick complementary rules. As used herein, the term “complementary sequences” means nucleic acid sequences that are substantially complementary, as may be assessed by the same nucleotide comparison set forth above, or as defined as being capable of hybridizing to the nucleic acid segment of a Chlamydia psittaci polynucleotide under relatively stringent conditions such as those described herein. Such sequences may encode the entire Chlamydia psittaci polypeptide or functional or non-functional fragments thereof.

Alternatively, the hybridizing segments may be shorter oligonucleotides. Sequences of 17 bases long should occur only once in the human genome and, therefore, suffice to specify a unique target sequence. Although shorter oligomers are easier to make and increase in vivo accessibility, numerous other factors are involved in determining the specificity of hybridization. Both binding affinity and sequence specificity of an oligonucleotide to its complementary target increases with increasing length. It is contemplated that exemplary oligonucleotides of 8, 9, 10, 11, 12, 13, 14, 15, 16, 17, 18, 19, 20, 25, 30, 35, 40, 45, 50, 55, 60, 65, 70, 75, 80, 85, 90, 95, 100 or more base pairs will be used, although others are contemplated. Longer polynucleotides encoding 250, 500, 1000, 1212, 1500, 2000, 2500, 3000 or 3500 bases and longer are contemplated as well. Such oligonucleotides will find use, for example, as probes in Southern and Northern blots and as primers in amplification reactions, or for vaccines.

Suitable hybridization conditions will be well known to those of skill in the art. In certain applications, for example, substitution of amino acids by site-directed mutagenesis, it is appreciated that lower stringency conditions are required. Under these conditions, hybridization may occur even though the sequences of probe and target strand are not perfectly complementary, but are mismatched at one or more positions. Conditions may be rendered less stringent by increasing salt concentration and decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Thus, hybridization conditions can be readily manipulated, and thus will generally be a method of choice depending on the desired results.

In other embodiments, hybridization may be achieved under conditions of, for example, 50 mM Tris-HCl (pH 8.3), 75 mM KCl, 3 MM MgCl₂, 10 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C. Formamide and SDS also may be used to alter the hybridization conditions.

One method of using probes and primers of the present invention is in the search for genes related to Chlamydia psittaci or, more particularly, homologs of Chlamydia psittaci from other species. Normally, the target DNA will be a genomic or cDNA library, although screening may involve analysis of RNA molecules. By varying the stringency of hybridization, and the region of the probe, different degrees of homology may be discovered.

Another way of exploiting probes and primers of the present invention is in site-directed, or site-specific mutagenesis. Site-specific mutagenesis is a technique useful in the preparation of individual peptides, or biologically functional equivalent proteins or peptides, through specific mutagenesis of the underlying DNA. The technique further provides a ready ability to prepare and test sequence variants, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 25 nucleotides in length is preferred, with about 5 to 10 residues on both sides of the junction of the sequence being altered.

The technique typically employs a bacteriophage vector that exists in both a single stranded and double stranded form. Typical vectors useful in site-directed mutagenesis include vectors such as the M13 phage. These phage vectors are commercially available and their use is generally well known to those skilled in the art. Double stranded plasmids are also routinely employed in site directed mutagenesis, which eliminates the step of transferring the gene of interest from a phage to a plasmid.

In general, site-directed mutagenesis is performed by first obtaining a single-stranded vector, or melting of two strands of a double stranded vector which includes within its sequence a DNA sequence encoding the desired protein. An oligonucleotide primer bearing the desired mutated sequence is synthetically prepared. This primer is then annealed with the single-stranded DNA preparation, taking into account the degree of mismatch when selecting hybridization conditions, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected that include recombinant vectors bearing the mutated sequence arrangement.

The preparation of sequence variants of the selected gene using site-directed mutagenesis is provided as a means of producing potentially useful species and is not meant to be limiting, as there are other ways in which sequence variants of genes may be obtained. For example, recombinant vectors encoding the desired gene may be treated with mutagenic agents, such as hydroxylamine, to obtain sequence variants.

C. Polypeptides and Antigens

For the purposes of the present invention a Chlamydia psittaci polypeptide used as an antigen may be a naturally-occurring Chlamydia psittaci polypeptide that has been extracted using protein extraction techniques well known to those of skill in the art. In particular embodiments, a Chlamydia psittaci antigen is identified by ELI and prepared in a pharmaceutically acceptable carrier for the vaccination of an animal against Chlamydia psittaci infection.

In alternative embodiments, the Chlamydia psittaci polypeptide or antigen may be a synthetic peptide. In still other embodiments, the peptide may be a recombinant peptide produced through molecular engineering techniques. The present section describes the methods and compositions involved in producing a composition of Chlamydia psittaci polypeptides for use as antigens in the present invention.

1. Chlamydia psittaci Polypeptides as Antigens

Section A describes methods for preparing a cloned Chlamydia psittaci library for ELI. Described also are methods for screening and identifying Chlamydia psittaci genes that confer protection against Chlamydia psittaci infection. Thus, Chlamydia psittaci polypeptide encoding genes or their corresponding cDNA identified in the present invention can be inserted into an appropriate cloning vehicle for the production of Chlamydia psittaci polypeptides as antigens for the present invention. In addition, sequence variants of the polypeptide can be prepared. These may, for instance, be minor sequence variants of the polypeptide that arise due to natural variation within the population or they may be homologues found in other species. They also may be sequences that do not occur naturally, but that are sufficiently similar that they function similarly and/or elicit an immune response that cross-reacts with natural forms of the polypeptide. Sequence variants can be prepared by standard methods of site-directed mutagenesis such as those described below in the following section.

Another synthetic or recombinant variation of a Chlamydia psittaci-antigen is a polyepitopic moiety comprising repeats of epitopic determinants found naturally on Chlamydia psittaci proteins. Such synthetic polyepitopic proteins can be made up of several homomeric repeats of any one Chlamydia psittaci protein epitope; or can comprise of two or more heteromeric epitopes expressed on one or several Chlamydia psittaci protein epitopes.

Amino acid sequence variants of the polypeptide can be substitutional, insertional or deletion variants. Deletion variants lack one or more residues of the native protein which are not essential for function or immunogenic activity, and are exemplified by the variants lacking a transmembrane sequence described above. Another common type of deletion variant is one lacking secretory signal sequences or signal sequences directing a protein to bind to a particular part of a cell.

Substitutional variants typically contain the exchange of one amino acid for another at one or more sites within the protein, and may be designed to modulate one or more properties of the polypeptide such as stability against proteolytic cleavage. Substitutions preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

Insertional variants include fusion proteins such as those used to allow rapid purification of the polypeptide and also can include hybrid proteins containing sequences from other proteins and polypeptides which are homologues of the polypeptide. For example, an insertional variant could include portions of the amino acid sequence of the polypeptide from one species, together with portions of the homologous polypeptide from another species. Other insertional variants can include those in which additional amino acids are introduced within the coding sequence of the polypeptide. These typically are smaller insertions than the fusion proteins described above and are introduced, for example, into a protease cleavage site.

In one embodiment, major antigenic determinants of the polypeptide may be identified by an empirical approach in which portions of the gene encoding the polypeptide are expressed in a recombinant host, and the resulting proteins tested for their ability to elicit an immune response. For example, the polymerase chain reaction (PCR) can be used to prepare a range of cDNAs encoding peptides lacking successively longer fragments of the C-terminus of the protein. The immunogenic activity of each of these peptides then identifies those fragments or domains of the polypeptide that are essential for this activity. Further experiments in which only a small number of amino acids are removed or added at each iteration then allows the location of other antigenic determinants of the polypeptide. Thus, the polymerase chain reaction, a technique for amplifying a specific segment of DNA via multiple cycles of denaturation-renaturation, using a thermostable DNA polymerase, deoxyribonucleotides and primer sequences is contemplated in the present invention (Mullis, 1990; Mullis et al., 1992).

Another embodiment for the preparation of the polypeptides according to the invention is the use of peptide mimetics. Mimetics are peptide-containing molecules that mimic elements of protein secondary structure. Because many proteins exert their biological activity via relatively small regions of their folded surfaces, their actions can be reproduced by much smaller designer (mimetic) molecules that retain the bioactive surfaces and have potentially improved pharmacokinetic/dynamic properties (Fairlie et al., 1998).

The underlying rationale behind the use of peptide mimetics is that the peptide backbone of proteins exists chiefly to orient amino acid side chains in such a way as to facilitate molecular interactions, such as those of antibody and antigen. However, unlike proteins, peptides often lack well defined three dimensional structure in aqueous solution and tend to be conformationally mobile. Progress has been made with the use of molecular constraints to stabilize the bioactive conformations. By affixing or incorporating templates that fix secondary and tertiary structures of small peptides, synthetic molecules (protein surface mimetics) can be devised to mimic the localized elements of protein structure that constitute bioactive surfaces. Methods for mimicking individual elements of secondary structure (helices, turns, strands, sheets) and for assembling their combinations into tertiary structures (helix bundles, multiple loops, helix-loop-helix motifs) have been reviewed (Fairlie et al., 1998; Moore, 1994).

Methods for predicting, preparing, modifying, and screening mimetic peptides are described in U.S. Pat. No. 5,933,819 and U.S. Pat. No. 5,869,451 (each specifically incorporated herein by reference). It is contemplated in the present invention, that peptide mimetics will be useful in screening modulators of an immune response.

Modifications and changes may be made in the structure of a gene and still obtain a functional molecule that encodes a protein or polypeptide with desirable characteristics. The following is a discussion based upon changing the amino acids of a protein to create an equivalent, or even an improved, second-generation molecule. The amino acid changes may be achieved by changing the codons of the DNA sequence, according to the following data.

For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid substitutions can be made in a protein sequence, and its underlying DNA coding sequence, and nevertheless obtain a protein with like properties. It is thus contemplated by the inventor that various changes may be made in the DNA sequences of genes without appreciable loss of their biological utility or activity. Table 1 shows the codons that encode particular amino acids.

In making such changes, the hydropathic index of amino acids may be considered. The importance of the hydropathic amino acid index in conferring interactive biologic function on a protein is generally understood in the art (Kyte & Doolittle, 1982).

It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

Each amino acid has been assigned a hydropathic index on the basis of their hydrophobicity and charge characteristics (Kyte & Doolittle, 1982), these are: Isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those which are within ±1 are particularly preferred, and those within ±0.5 are even more particularly preferred.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine *−0.5); cysteine (−1.0); methionine (−1.3); valine (−1.5); leucine (−1.8); isoleucine (−1.8); tyrosine (−2.3); phenylalanine (−2.5); tryptophan (−3.4).

It is understood that an amino acid can be substituted for another having a similar hydrophilicity value and still obtain a biologically equivalent and immunologically equivalent protein. In such changes, the substitution of amino acids whose hydrophilicity values are within ±2 is preferred, those that are within +1 are particularly preferred, and those within +0.5 are even more particularly preferred.

As outlined above, amino acid substitutions generally are based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine.

2. Synthetic Polypeptides

Contemplated in the present invention are Chlamydia psittaci proteins and related peptides for use as antigens. In certain embodiments, the synthesis of a Chlamydia psittaci peptide fragment is considered. The peptides of the invention can be synthesized in solution or on a solid support in accordance with conventional techniques. Various automatic synthesizers are commercially available and can be used in accordance with known protocols. See, for example, Stewart and Young, (1984); Tam et al., (1983); Merrifield, (1986); and Barany and Merrifield (1979), each incorporated herein by reference. Alternatively, recombinant DNA technology may be employed wherein a nucleotide sequence which encodes a peptide of the invention is inserted into an expression vector, transformed or transfected into an appropriate host cell and cultivated under conditions suitable for expression.

3. Chlamydia psittaci Polypeptide/Antigen Purification

Chlamydia psittaci polypeptides of the present invention are used as antigens for inducing a protective immune response in an animal and for the preparation of anti-Chlamydia psittaci antibodies. Thus, certain aspects of the present invention concern the purification, and in particular embodiments, the substantial purification, of a Chlamydia psittaci polypeptide that is described herein above. The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50% or more of the proteins in the composition.

Various methods for quantifying the degree of purification of the protein or peptide will be known to those of skill in the art in light of the present disclosure. These include, for example, determining the specific activity of an active fraction, or assessing the number of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater-fold purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

High Performance Liquid Chromatography (HPLC) is characterized by a very rapid separation with extraordinary resolution of peaks. This is achieved by the use of very fine particles and high pressure to maintain and adequate flow rate. Separation can be accomplished in a matter of minutes, or a most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, etc.).

D. Gene Delivery

In certain embodiments of the invention, an expression construct comprising a Chlamydia psittaci gene or polynucleotide segment under the control of a heterologous promoter operable in eukaryotic cells is provided. The general approach in certain aspects of the present invention is to provide a cell with an expression construct encoding a specific Chlamydia psittaci protein, polypeptide or peptide fragment, thereby permitting the antigenic expression of the protein, polypeptide or peptide fragment to take effect in the cell. Following delivery of the expression construct, the protein, polypeptide or peptide fragment encoded by the expression construct is synthesized by the transcriptional and translational machinery of the cell, as well as any that may be provided by the expression construct.

Viral and non-viral vector systems are the two predominate categories for the delivery of an expression construct encoding a therapeutic protein, polypeptide, polypeptide fragment. Both vector systems are described in the following sections. There also are two primary approaches utilized in the delivery of an expression construct for the purposes of gene therapy; either indirect, ex vivo methods or direct, in vivo methods. Ex vivo gene transfer comprises vector modification of (host) cells in culture and the administration or transplantation of the vector modified cells to a gene therapy recipient. In vivo gene transfer comprises direct introduction of the vector (e.g., injection, inhalation) into the target source or therapeutic gene recipient.

In certain embodiments of the invention, the nucleic acid encoding the gene or polynucleotide may be stably integrated into the genome of the cell. In yet further embodiments, the nucleic acid may be stably or transiently maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and/or where in the cell the nucleic acid remains is dependent on the type of vector employed. The following gene delivery methods provide the framework for choosing and developing the most appropriate gene delivery system for a preferred application.

1. Non-Viral Polynucleotide Delivery

In one embodiment of the invention, a polynucleotide expression construct consists of naked recombinant DNA or plasmids. In preferred embodiments of the invention, a Chlamydia psittaci polynucleotide is administered to a subject via injection and/or particle bombardment (e.g., a gene gun). Thus, in one preferred embodiment, polynucleotide expression constructs are transferred into cells by accelerating DNA-coated microprojectiles to a high velocity, allowing the DNA-coated microprojectiles to pierce cell membranes and enter cells. In another preferred embodiment, polynucleotides are administered to a subject by injection. Injection of a polynucleotide expression construct may be given by intramuscular, intravenous, subcutaneous, ir intraperitoneal injection, as long as the polynucleotide expression construct can effectively be delivered to a desired target.

a. Particle Bombardment

Particle Bombardment depends on the ability to accelerate DNA-coated microprojectiles to a high velocity allowing them to pierce cell membranes and enter cells without killing them (Klein et al., 1987). Several devices for accelerating small particles have been developed. The most commonly used forms rely on high-pressure helium gas (Sanford et al., 1991), of which one of the present inventors is a co-inventor. The microprojectiles used have consisted of biologically inert substances such as tungsten or gold beads.

For microprojectile bombardment transformation using the constructs of the instant invention, both physical and biological parameters may be optimized. Physical factors are those that involve manipulating the DNA/microprojectile precipitate or those that affect the flight and velocity of either the macro- or microprojectiles. Biological factors include all steps involved in manipulation of cells before and immediately after bombardment, such as the osmotic adjustment of target cells to help alleviate the trauma associated with bombardment, the orientation of an immature embryo or other target tissue relative to the particle trajectory, and also the nature of the transforming DNA, such as linearized DNA or intact supercoiled plasmids.

Accordingly, it is contemplated that one may wish to adjust various bombardment parameters in small scale studies to fully optimize the conditions. One may particularly wish to adjust physical parameters such as DNA concentration, gap distance, flight distance, tissue distance, and helium pressure. It is further contemplated that the grade of helium may effect transformation efficiency. One also may optimize the trauma reduction factors (TRFs) by modifying conditions which influence the physiological state of the recipient cells and which may therefore influence transformation and integration efficiencies. For example, the osmotic state, tissue hydration and the subculture stage or cell cycle of the recipient cells may be adjusted for optimum transformation.

Other physical factors include those that involve manipulating the DNA/microprojectile precipitate or those that affect the flight and velocity of either the macro- or microprojectiles. Biological factors include all steps involved in manipulation of cells immediately before and after bombardment. The pre-bombardment culturing conditions, such as osmotic environment, the bombardment parameters, and the plasmid configuration have been adjusted to yield the maximum numbers of stable transformants.

For microprojectile bombardment, one will attach (i.e., “coat”) DNA to the microprojectiles such that it is delivered to recipient cells in a form suitable for transformation thereof. In this respect, at least some of the transforming DNA must be available to the target cell for transformation to occur, while at the same time during delivery the DNA must be attached to the microprojectile. Therefore, availability of the transforming DNA from the microprojectile may comprise the physical reversal of interactions between transforming DNA and the microprojectile following delivery of the microprojectile to the target cell. This need not be the case, however, as availability to a target cell may occur as a result of breakage of unbound segments of DNA or of other molecules which comprise the physical attachment to the microprojectile. Availability may further occur as a result of breakage of bonds between the transforming DNA and other molecules, which are either directly or indirectly attached to the microprojectile. It is further contemplated that transformation of a target cell may occur by way of direct illegitimate or homology-dependent recombination between the transforming DNA and the genomic DNA of the recipient cell. Therefore, as used herein, a “coated” microprojectile will be one which is capable of being used to transform a target cell, in that the transforming DNA will be delivered to the target cell, yet will be accessible to the target cell such that transformation may occur.

Any technique for coating microprojectiles which allows for delivery of transforming DNA to the target cells may be used. Methods for coating microprojectiles which have been demonstrated to work well with the current invention have been specifically disclosed herein. DNA may be bound to microprojectile particles using alternative techniques, however. For example, particles may be coated with streptavidin and DNA end labeled with long chain thiol cleavable biotinylated nucleotide chains. The DNA adheres to the particles due to the streptavidin-biotin interaction, but is released in the cell by reduction of the thiol linkage through reducing agents present in the cell.

Alternatively, particles may be prepared by functionalizing the surface of a gold oxide particle, providing free amine groups. DNA, having a strong negative charge, binds to the functionalized particles. Furthermore, charged particles may be deposited in controlled arrays on the surface of mylar flyer disks used in the PDS-1000 Biolistics device, thereby facilitating controlled distribution of particles delivered to target tissue.

b. Other Non-Viral Methods of Polynucleotide Delivery

Transfer of a cloned expression construct in the present invention also may be performed by any of the methods which physically or chemically permeabilize the cell membrane (e.g., calcium phosphate precipitation, DEAE-dextran, electroporation, direct microinjection, DNA-loaded liposomes and lipofectamine-DNA complexes, cell sonication, gene bombardment using high velocity microprojectiles and receptor-mediated transfection.

In certain embodiments, the use of lipid formulations and/or nanocapsules is contemplated for the introduction of a Chlamydia psittaci polynucleotide or polypeptide, or a gene therapy vector into host cells.

Nanocapsules can generally entrap compounds in a stable and/or reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use in the present invention, and/or such particles may be easily made.

In a preferred embodiment of the invention, the polynucleotide or polypeptide may be associated with a lipid. The polynucleotide or polypeptide associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. The lipid or lipid/polynucleotide or polypeptide associated compositions of the present invention are not limited to any particular structure in solution. For example, they may be present in a bilayer structure, as micelles, or with a “collapsed” structure. They may also simply be interspersed in a solution, possibly forming aggregates which are not uniform in either size or shape.

Lipids suitable for use according to the present invention can be obtained from commercial sources. For example, dimyristyl phosphatidylcholine (“DMPC”) can be obtained from Sigma Chemical Co., dicetyl phosphate (“DCP”) is obtained from K & K Laboratories (Plainview, N.Y.); cholesterol (“Chol”) is obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Preferably, chloroform is used as the only solvent since it is more readily evaporated than methanol.

“Liposome” is a generic term encompassing a variety of single and multilamellar lipid vehicles formed by the generation of enclosed lipid bilayers or aggregates. Liposomes may be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). However, the present invention also encompasses compositions that have different structures in solution than the normal vesicular structure. For example, the lipids may assume a micellar structure or merely exist as non-uniform aggregates of lipid molecules. Also contemplated are lipofectamine-nucleic acid complexes.

Liposomes within the scope of the present invention can be prepared in accordance with known laboratory procedures, for example: the method of Bangham et al. (1965), the contents of which are incorporated herein by reference; the method of Gregoriadis, as described in DRUG CARRIERS IN BIOLOGY AND MEDICINE, G. Gregoriadis ed. (1979) pp. 287-341, the contents of which are incorporated herein by reference; the method of Deamer and Uster (1983), the contents of which are incorporated by reference; and the reverse-phase evaporation method as described by Szoka and Papahadjopoulos (1978).

Other vector delivery systems which can be employed to deliver a nucleic acid encoding a therapeutic gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu, 1993).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, 1987) and transferring (Wagner et al., 1990). Recently, a synthetic neoglycoprotein, which recognizes the same receptor as ASOR, has been used as a gene delivery vehicle (Ferkol et al., 1993; Perales et al., 1994) and epidermal growth factor (EGF) has also been used to deliver genes to squamous carcinoma cells (Myers, EPO 0273085).

In other embodiments, the delivery vehicle may comprise a ligand and a liposome. For example, Nicolau et al. (1987) employed lactosyl-ceramide, a galactose-terminal asialganglioside, incorporated into liposomes and observed an increase in the uptake of the insulin gene by hepatocytes. Thus, it is feasible that a nucleic acid encoding a therapeutic gene also may be specifically delivered into a cell type such as prostate, epithelial or tumor endothelial cells, by any number of receptor-ligand systems with or without liposomes. For example, the human prostate-specific antigen (Watt et al., 1986) may be used as the receptor for mediated delivery of a nucleic acid in prostate tissue.

In another embodiment of the invention, the expression construct may simply consist of naked recombinant DNA or plasmids. Transfer of the construct may be performed by any of the methods mentioned above which physically or chemically pertneabilize the cell membrane. This is applicable particularly for transfer in vitro, however, it may be applied for in vivo use as well. Dubensky et al. (1984) successfully injected polyomavirus DNA in the form of CaPO₄ precipitates into liver and spleen of adult and newborn mice demonstrating active viral replication and acute infection. Benvenisty and Neshif (1986) also demonstrated that direct intraperitoneal injection of CaPO₄ precipitated plasmids results in expression of the transfected genes. It is envisioned that DNA encoding a Chlamydia psittaci gene or polynucleotide of interest may also be transferred in a similar manner in vivo and express the gene or polynucleotide product.

2. Viral Vectors

In certain embodiments, it is contemplated that a Chlamydia psittaci gene or polynucleotide that confers immune resistance to infection may be delivered by a viral vector. The capacity of certain viral vectors to efficiently infect or enter cells, to integrate into a host cell genome and stably express viral genes, have led to the development and application of a number of different viral vector systems (Robbins et al., 1998). Viral systems are currently being developed for use as vectors for ex vivo and in vivo gene transfer. For example, adenovirus, herpes-simple virus, retrovirus and adeno-associated virus vectors are being evaluated currently for treatment of diseases such as cancer, cystic fibrosis, Gaucher disease, renal disease and arthritis (Robbins and Ghivizzani, 1998; Imai et al., 1998; U.S. Pat. No. 5,670,488). The various viral vectors described below, present specific advantages and disadvantages, depending on the particular gene-therapeutic application.

a. Adenoviral Vectors

In particular embodiments, an adenoviral expression vector is contemplated for the delivery of expression constructs. “Adenovirus expression vector” is meant to include those constructs containing adenovirus sequences sufficient to (a) support packaging of the construct and (b) to ultimately express a tissue or cell-specific construct that has been cloned therein.

Adenoviruses comprise linear double stranded DNA, with a genome ranging from 30 to 35 kb in size (Reddy et al., 1998; Morrison et al., 1997; Chillon et al., 1999). An adenovirus expression vector according to the present invention comprises a genetically engineered form of the adenovirus. Advantages of adenoviral gene transfer include the ability to infect a wide variety of cell types, including non-dividing cells, a mid-sized genome, ease of manipulation, high infectivity and they can be grown to high titers (Wilson, 1996). Further, adenoviral infection of host cells does not result in chromosomal integration because adenoviral DNA can replicate in an episomal manner, without potential genotoxicity associated with other viral vectors. Adenoviruses also are structurally stable (Marienfeld et al., 1999) and no genome rearrangement has been detected after extensive amplification (Parks et al., 1997; Bett et al., 1993).

Salient features of the adenovirus genome are an early region (E1, E2, E3 and E4 genes), an intermediate region (pIX gene, Iva2 gene), a late region (L1, L2, L3, L4 and L5 genes), a major late promoter (MLP), inverted-terminal-repeats (ITRs) and a ψ sequence (Zheng, et al., 1999; Robbins et al., 1998; Graham and Prevec, 1995). The early genes E1, E2, E3 and E4 are expressed from the virus after infection and encode polypeptides that regulate viral gene expression, cellular gene expression, viral replication, and inhibition of cellular apoptosis. Further on during viral infection, the MLP is activated, resulting in the expression of the late (L) genes, encoding polypeptides required for adenovirus encapsidation. The intermediate region encodes components of the adenoviral capsid. Adenoviral inverted terminal repeats (ITRs; 100-200 bp in length), are cis elements, function as origins of replication and are necessary for viral DNA replication. The ψ sequence is required for the packaging of the adenoviral genome.

A common approach for generating an adenoviruses for use as a gene transfer vector is the deletion of the E1 gene (E1⁻), which is involved in the induction of the E2, E3 and E4 promoters (Graham and Prevec, 1995). Subsequently, a therapeutic gene or genes can be inserted recombinantly in place of the E1 gene, wherein expression of the therapeutic gene(s) is driven by the E1 promoter or a heterologous promoter. The E1⁻, replication-deficient virus is then proliferated in a “helper” cell line that provides the E1 polypeptides in trans (e.g., the human embryonic kidney cell line 293). Thus, in the present invention it may be convenient to introduce the transforming construct at the position from which the E1-coding sequences have been removed. However, the position of insertion of the construct within the adenovirus sequences is not critical to the invention. Alternatively, the E3 region, portions of the E4 region or both may be deleted, wherein a heterologous nucleic acid sequence under the control of a promoter operable in eukaryotic cells is inserted into the adenovirus genome for use in gene transfer (U.S. Pat. No. 5,670,488; U.S. Pat. No. 5,932,210, each specifically incorporated herein by reference).

Although adenovirus based vectors offer several unique advantages over other vector systems, they often are limited by vector immunogenicity, size constraints for insertion of recombinant genes and low levels of replication. The preparation of a recombinant adenovirus vector deleted of all open reading frames, comprising a full length dystrophin gene and the terminal repeats required for replication (Haecker et al., 1997) offers some potentially promising advantages to the above mentioned adenoviral shortcomings. The vector was grown to high titer with a helper virus in 293 cells and was capable of efficiently transducing dystrophin in mdx mice, in myotubes in vitro and muscle fibers in vivo. Helper-dependent viral vectors are discussed below.

A major concern in using adenoviral vectors is the generation of a replication-competent virus during vector production in a packaging cell line or during gene therapy treatment of an individual. The generation of a replication-competent virus could pose serious threat of an unintended viral infection and pathological consequences for the patient. Armentano et al., describe the preparation of a replication-defective adenovirus vector, claimed to eliminate the potential for the inadvertent generation of a replication-competent adenovirus (U.S. Pat. No. 5,824,544, specifically incorporated herein by reference). The replication-defective adenovirus method comprises a deleted El region and a relocated protein 1×gene, wherein the vector expresses a heterologous, mammalian gene.

Other than the requirement that the adenovirus vector be replication defective, or at least conditionally defective, the nature of the adenovirus vector is not believed to be crucial to the successful practice of the invention. The adenovirus may be of any of the 42 different known serotypes and/or subgroups A-F. Adenovirus type 5 of subgroup C is the preferred starting material in order to obtain the conditional replication-defective adenovirus vector for use in the present invention. This is because adenovirus type 5 is a human adenovirus about which a great deal of biochemical and genetic information is known, and it has historically been used for most constructions employing adenovirus as a vector.

As stated above, the typical vector according to the present invention is replication defective and will not have an adenovirus El region. Adenovirus growth and manipulation is known to those of skill in the art, and exhibits broad host range in vitro and in vivo (U.S. Pat. No. 5,670,488; U.S. Pat. No. 5,932,210; U.S. Pat. No. 5,824,54). This group of viruses can be obtained in high titers, e.g., 10⁹ to 10¹¹ plaque-forming units per ml, and they are highly infective. The life cycle of adenovirus does not require integration into the host cell genome. The foreign genes delivered by adenovirus vectors are episomal and, therefore, have low genotoxicity to host cells. Many experiments, innovations, preclinical studies and clinical trials are currently under investigation for the use of adenoviruses as gene delivery vectors. For example, adenoviral gene delivery-based gene therapies are being developed for liver diseases (Han et al., 1999), psychiatric diseases (Lesch, 1999), neurological diseases (Smith, 1998; Hermens and Verhaagen, 1998), coronary diseases (Feldman et al., 1996), muscular diseases (Petrof, 1998), gastrointestinal diseases (Wu, 1998) and various cancers such as colorectal (Fujiwara and Tanaka, 1998; Dorai et al., 1999), pancreatic (Carrion et al., 1999), bladder (Irie et al., 1999), head and neck (Blackwell et al., 1999), breast (Stewart et al., 1999), lung (Batra et al., 1999) and ovarian (Vanderkwaak et al., 1999).

b. Retroviral Vectors

In certain embodiments of the invention, the use of retroviruses for gene delivery are contemplated. Retroviruses are RNA viruses comprising an RNA genome. When a host cell is infected by a retrovirus, the genomic RNA is reverse transcribed into a DNA intermediate which is integrated into the chromosomal DNA of infected cells. This integrated DNA intermediate is referred to as a provirus. A particular advantage of retroviruses is that they can stably infect dividing cells with a gene of interest (e.g., a therapeutic gene) by integrating into the host DNA, without expressing immunogenic viral proteins. Theoretically, the integrated retroviral vector will be maintained for the life of the infected host cell, expressing the gene of interest.

The retroviral genome and the proviral DNA have three genes: gag, pol, and env, which are flanked by two long terminal repeat (LTR) sequences. The gag gene encodes the internal structural (matrix, capsid, and nucleocapsid) proteins; the pol gene encodes the RNA-directed DNA polymerase (reverse transcriptase) and the env gene encodes viral envelope glycoproteins. The 5′ and 3′ LTRs serve to promote transcription and polyadenylation of the virion RNAs. The LTR contains all other cis-acting sequences necessary for viral replication.

A recombinant retrovirus of the present invention may be genetically modified in such a way that some of the structural, infectious genes of the native virus have been removed and replaced instead with a nucleic acid sequence to be delivered to a target cell (U.S. Pat. No. 5,858,744; U.S. Pat. No. 5,739,018, each incorporated herein by reference). After infection of a cell by the virus, the virus injects its nucleic acid into the cell and the retrovirus genetic material can integrate into the host cell genome. The transferred retrovirus genetic material is then transcribed and translated into proteins within the host cell. As with other viral vector systems, the generation of a replication-competent retrovirus during vector production or during therapy is a major concern. Retroviral vectors suitable for use in the present invention are generally defective retroviral vectors that are capable of infecting the target cell, reverse transcribing their RNA genomes, and integrating the reverse transcribed DNA into the target cell genome, but are incapable of replicating within the target cell to produce infectious retroviral particles (e.g., the retroviral genome transferred into the target cell is defective in gag, the gene encoding virion structural proteins, and/or in pol, the gene encoding reverse transcriptase). Thus, transcription of the provirus and assembly into infectious virus occurs in the presence of an appropriate helper virus or in a cell line containing appropriate sequences enabling encapsidation without coincident production of a contaminating helper virus.

The growth and maintenance of retroviruses is known in the art (U.S. Pat. No. 5,955,331; U.S. Pat. No. 5,888,502, each specifically incorporated herein by reference). Nolan et al. describe the production of stable high titre, helper-free retrovirus comprising a heterologous gene (U.S. Pat. No. 5,830,725, specifically incorporated herein by reference). Methods for constructing packaging cell lines useful for the generation of helper-free recombinant retroviruses with amphoteric or ecotrophic host ranges, as well as methods of using the recombinant retroviruses to introduce a gene of interest into eukaryotic cells in vivo and in vitro are contemplated in the present invention (U.S. Pat. No. 5,955,331).

Currently, the majority of all clinical trials for vector mediated gene delivery use murine leukemia virus (MLV)-based retroviral vector gene delivery (Robbins et al., 1998; Miller et al., 1993). Disadvantages of retroviral gene delivery includes a requirement for ongoing cell division for stable infection and a coding capacity that prevents the delivery of large genes. However, recent development of vectors such as lentivirus (e.g., HIV), simian immunodeficiency virus (SIV) and equine infectious-anemia virus (EIAV), which can infect certain non-dividing cells, potentially allow the in vivo use of retroviral vectors for gene therapy applications (Amado and Chen, 1999; Klimatcheva et al., 1999; White et al., 1999; Case et al., 1999). For example, HIV-based vectors have been used to infect non-dividing cells such as neurons (Takashi et al., 1999; Miyake et al., 1999), islets (Leibowitz et al., 1999) and muscle cells (Johnston et al., 1999). The therapeutic delivery of genes via retroviruses are currently being assessed for the treatment of various disorders such as inflammatory disease (Moldawer et al., 1999),

AIDS (Amado et al., 1999; Engel and Kohn, 1999), cancer (Clay et al., 1999), cerebrovascular disease (Weihl et al., 1999) and hemophilia (Kay, 1998).

C. Herpes-Simplex Viral Vectors

Herpes simplex virus (HSV) type I and type II contain a double-stranded, linear DNA genome of approximately 150 kb, encoding 70-80 genes. Wild type HSV are able to infect cells lytically and to establish latency in certain cell types (e.g., neurons).

Similar to adenovirus, HSV also can infect a variety of cell types including muscle (Yeung et al., 1999), ear (Derby et al., 1999), eye (Kaufman et al., 1999), tumors (Yoon et al., 1999; Howard et al., 1999), lung (Kohut et al., 1998), neuronal (Gamido et al., 1999; Lachmann and Efstathiou, 1999), liver (Miytake et al., 1999; Kooby et al., 1999) and pancreatic islets (Rabinovitch et al., 1999).

HSV viral genes are transcribed by cellular RNA polymerase II and are temporally regulated, resulting in the transcription and subsequent synthesis of gene products in roughly three discernable phases or kinetic classes. These phases of genes are referred to as the Immediate Early (IE) or alpha genes, Early (E) or beta genes and Late (L) or gamma genes. Immediately following the arrival of the genome of a virus in the nucleus of a newly infected cell, the IE genes are transcribed. The efficient expression of these genes does not require prior viral protein synthesis. The products of IE genes are required to activate transcription and regulate the remainder of the viral genome.

For use in therapeutic gene delivery, HSV must be rendered replication-defective. Protocols for generating replication-defective HSV helper virus-free cell lines have been described (U.S. Pat. No. 5,879,934; U.S. Pat. No. 5,851,826, each specifically incorporated herein by reference in its entirety). One IE protein, Infected Cell Polypeptide 4 (ICP4), also known as alpha 4 or Vmw175, is absolutely required for both virus infectivity and the transition from IE to later transcription. Thus, due to its complex, multifunctional nature and central role in the regulation of HSV gene expression, ICP4 has typically been the target of HSV genetic studies.

Phenotypic studies of HSV viruses deleted of ICP4 indicate that such viruses will be potentially useful for gene transfer purposes (Krisky et al., 1998a). One property of viruses deleted for ICP4 that makes them desirable for gene transfer is that they only express the five other IE genes: ICP0, ICP6, ICP27, ICP22 and ICP47 (DeLuca et al., 1985), without the expression of viral genes encoding proteins that direct viral DNA synthesis, as well as the structural proteins of the virus. This property is desirable for minimizing possible deleterious effects on host cell metabolism or an immune response following gene transfer. Further deletion of IE genes ICP22 and ICP27, in addition to ICP4, substantially improve reduction of HSV cytotoxicity and prevented early and late viral gene expression (Krisky et al., 1998b).

The therapeutic potential of HSV in gene transfer has been demonstrated in various in vitro model systems and in vivo for diseases such as Parkinson's (Yamada et al., 1999), retinoblastoma (Hayashi et al., 1999), intracerebral and intradermal tumors (Moriuchi et al., 1998), B cell malignancies (Suzuki et al., 1998), ovarian cancer (Wang et al., 1998) and Duchenne muscular dystrophy (Huard et al., 1997).

d. Adeno-Associated Viral Vectors

Adeno-associated virus (AAV), a member of the parvovirus family, is a human virus that is increasingly being used for gene delivery therapeutics. AAV has several advantageous features not found in other viral systems. First, AAV can infect a wide range of host cells, including non-dividing cells. Second, AAV can infect cells from different species. Third, AAV has not been associated with any human or animal disease and does not appear to alter the biological properties of the host cell upon integration. For example, it is estimated that 80-85% of the human population has been exposed to AAV. Finally, AAV is stable at a wide range of physical and chemical conditions which lends itself to production, storage and transportation requirements.

The AAV genome is a linear, single-stranded DNA molecule containing 4681 nucleotides. The AAV genome generally comprises an internal non-repeating genome flanked on each end by inverted terminal repeats (ITRs) of approximately 145 bp in length. The ITRs have multiple functions, including origins of DNA replication, and as packaging signals for the viral genome. The internal non-repeated portion of the genome includes two large open reading frames, known as the AAV replication (rep) and capsid (cap) genes. The rep and cap genes code for viral proteins that allow the virus to replicate and package the viral genome into a virion. A family of at least four viral proteins are expressed from the AAV rep region, Rep 78, Rep 68, Rep 52, and Rep 40, named according to their apparent molecular weight. The AAV cap region encodes at least three proteins, VP1, VP2, and VP3.

AAV is a helper-dependent virus requiring co-infection with a helper virus (e.g., adenovirus, herpesvirus or vaccinia) in order to form AAV virions. In the absence of co-infection with a helper virus, AAV establishes a latent state in which the viral genome inserts into a host cell chromosome, but infectious virions are not produced. Subsequent infection by a helper virus “rescues” the integrated genome, allowing it to replicate and package its genome into infectious AAV virions. Although AAV can infect cells from different species, the helper virus must be of the same species as the host cell (e.g., human AAV will replicate in canine cells co-infected with a canine adenovirus).

AAV has been engineered to deliver genes of interest by deleting the internal non-repeating portion of the AAV genome and inserting a heterologous gene between the ITRs. The heterologous gene may be functionally linked to a heterologous promoter (constitutive, cell-specific, or inducible) capable of driving gene expression in target cells. To produce infectious recombinant AAV (rAAV) containing a heterologous gene, a suitable producer cell line is transfected with a rAAV vector containing a heterologous gene. The producer cell is concurrently transfected with a second plasmid harboring the AAV rep and cap genes under the control of their respective endogenous promoters or heterologous promoters. Finally, the producer cell is infected with a helper virus.

Once these factors come together, the heterologous gene is replicated and packaged as though it were a wild-type AAV genome. When target cells are infected with the resulting rAAV virions, the heterologous gene enters and is expressed in the target cells. Because the target cells lack the rep and cap genes and the adenovirus helper genes, the rAAV cannot further replicate, package or form wild-type AAV.

The use of helper virus, however, presents a number of problems. First, the use of adenovirus in a rAAV production system causes the host cells to produce both rAAV and infectious adenovirus. The contaminating infectious adenovirus can be inactivated by heat treatment (56.degree. C. for 1 hour). Heat treatment, however, results in approximately a 50% drop in the titer of functional rAAV virions. Second, varying amounts of adenovirus proteins are present in these preparations. For example, approximately 50% or greater of the total protein obtained in such rAAV virion preparations is free adenovirus fiber protein. If not completely removed, these adenovirus proteins have the potential of eliciting an immune response from the patient. Third, AAV vector production methods which employ a helper virus require the use and manipulation of large amounts of high titer infectious helper virus, which presents a number of health and safety concerns, particularly in regard to the use of a herpesvirus. Fourth, concomitant production of helper virus particles in rAAV virion producing cells diverts large amounts of host cellular resources away from rAAV virion production, potentially resulting in lower rAAV virion yields.

e. Other Viral Vectors

The development and utility of viral vectors for gene delivery is constantly improving and evolving. Other viral vectors such as poxyirus; e.g., vaccinia virus (Gnant et al., 1999; Gnant et al., 1999), alpha virus; e.g., sindbis virus, Semliki forest virus (Lundstrom, 1999), reovirus (Coffey et al., 1998) and influenza A virus (Neumann et al., 1999) are contemplated for use in the present invention and may be selected according to the requisite properties of the target system.

In certain embodiments, vaccinia viral vectors are contemplated for use in the present invention. Vaccinia virus is a particularly useful eukaryotic viral vector system for expressing heterologous genes. For example, when recombinant vaccinia virus is properly engineered, the proteins are synthesized, processed and transported to the plasma membrane. Vaccinia viruses as gene delivery vectors have recently been demonstrated to transfer genes to human tumor cells, e.g., EMAP-II (Gnant et al., 1999), inner ear (Derby et al., 1999), glioma cells, e.g., p53 (Timiryasova et al., 1999) and various mammalian cells, e.g., P-450 (U.S. Pat. No. 5,506,138). The preparation, growth and manipulation of vaccinia viruses are described in U.S. Pat. No. 5,849,304 and U.S. Pat. No. 5,506,138 (each specifically incorporated herein by reference).

In other embodiments, sindbis viral vectors are contemplated for use in gene delivery. Sindbis virus is a species of the alphavirus genus (Garoff and Li, 1998) which includes such important pathogens as Venezuelan, Western and Eastern equine encephalitis viruses (Sawai et al., 1999; Mastrangelo et al., 1999). In vitro, sindbis virus infects a variety of avian, mammalian, reptilian, and amphibian cells. The genome of sindbis virus consists of a single molecule of single-stranded RNA, 11,703 nucleotides in length. The genomic RNA is infectious, is capped at the 5′ terminus and polyadenylated at the 3′ terminus, and serves as mRNA. Translation of a vaccinia virus 26S mRNA produces a polyprotein that is cleaved co- and post-translationally by a combination of viral and presumably host-encoded proteases to give the three virus structural proteins, a capsid protein (C) and the two envelope glycoproteins (E1 and PE2, precursors of the virion E2).

Three features of sindbis virus suggest that it would be a useful vector for the expression of heterologous genes. First, its wide host range, both in nature and in the laboratory. Second, gene expression occurs in the cytoplasm of the host cell and is rapid and efficient. Third, temperature-sensitive mutations in RNA synthesis are available that may be used to modulate the expression of heterologous coding sequences by simply shifting cultures to the non-permissive temperature at various time after infection. The growth and maintenance of sindbis virus is known in the art (U.S. Pat. No. 5,217,879, specifically incorporated herein by reference).

f. Chimeric Viral Vectors

Chimeric or hybrid viral vectors are being developed for use in therapeutic gene delivery and are contemplated for use in the present invention. Chimeric poxyiral/retroviral vectors (Holzer et al., 1999), adenoviral/retroviral vectors (Feng et al., 1997; Bilbao et al., 1997; Caplen et al., 1999) and adenoviral/adeno-associated viral vectors (Fisher et al., 1996; U.S. Pat. No. 5,871,982) have been described.

These “chimeric” viral gene transfer systems can exploit the favorable features of two or more parent viral species. For example, Wilson et al., provide a chimeric vector construct which comprises a portion of an adenovirus, AAV 5′ and 3′ ITR sequences and a selected transgene, described below (U.S. Pat. No. 5,871,983, specifically incorporate herein by reference).

The adenovirus/AAV chimeric virus uses adenovirus nucleic acid sequences as a shuttle to deliver a recombinant AAV/transgene genome to a target cell. The adenovirus nucleic acid sequences employed in the hybrid vector can range from a minimum sequence amount, which requires the use of a helper virus to produce the hybrid virus particle, to only selected deletions of adenovirus genes, which deleted gene products can be supplied in the hybrid viral production process by a selected packaging cell. At a minimum, the adenovirus nucleic acid sequences employed in the pAdA shuttle vector are adenovirus genomic sequences from which all viral genes are deleted and which contain only those adenovirus sequences required for packaging adenoviral genomic DNA into a preformed capsid head. More specifically, the adenovirus sequences employed are the cis-acting 5′ and 3′ inverted terminal repeat (ITR) sequences of an adenovirus (which function as origins of replication) and the native 5′ packaging/enhancer domain, that contains sequences necessary for packaging linear Ad genomes and enhancer elements for the El promoter. The adenovirus sequences may be modified to contain desired deletions, substitutions, or mutations, provided that the desired function is not eliminated.

The AAV sequences useful in the above chimeric vector are the viral sequences from which the rep and cap polypeptide encoding sequences are deleted. More specifically, the AAV sequences employed are the cis-acting 5′ and 3′ inverted terminal repeat (ITR) sequences. These chimeras are characterized by high titer transgene delivery to a host cell and the ability to stably integrate the transgene into the host cell chromosome (U.S. Pat. No. 5,871,983, specifically incorporate herein by reference). In the hybrid vector construct, the AAV sequences are flanked by the selected adenovirus sequences discussed above. The 5′ and 3′ AAV ITR sequences themselves flank a selected transgene sequence and associated regulatory elements, described below. Thus, the sequence formed by the transgene and flanking 5′ and 3′ AAV sequences may be inserted at any deletion site in the adenovirus sequences of the vector. For example, the AAV sequences are desirably inserted at the site of the deleted E1a/E1b genes of the adenovirus. Alternatively, the AAV sequences may be inserted at an E3 deletion, E2a deletion, and so on. If only the adenovirus 5′ ITR/packaging sequences and 3′ ITR sequences are used in the hybrid virus, the AAV sequences are inserted between them.

The transgene sequence of the vector and recombinant virus can be a gene, a nucleic acid sequence or reverse transcript thereof, heterologous to the adenovirus sequence, which encodes a protein, polypeptide or peptide fragment of interest. The transgene is operatively linked to regulatory components in a manner which permits transgene transcription. The composition of the transgene sequence will depend upon the use to which the resulting hybrid vector will be put. For example, one type of transgene sequence includes a therapeutic gene which expresses a desired gene product in a host cell. These therapeutic genes or nucleic acid sequences typically encode products for administration and expression in a patient in vivo or ex vivo to replace or correct an inherited or non-inherited genetic defect or treat an epigenetic disorder or disease.

E. Antibodies Against Chlamydia psittaci Proteins.

In another aspect, the present invention provides antibody compositions that are immunoreactive with a Chlamydia psittaci polypeptide of the present invention, or any portion thereof.

An antibody can be a polyclonal or a monoclonal antibody. An antibody may also be monovalent or bivalent. A prototype antibody is an immunoglobulin composed by four polypeptide chains, two heavy and two light chains, held together by disulfide bonds. Each pair of heavy and light chains forms an antigen binding site, also defined as complementarity-determining region (CDR). Therefore, the prototype antibody has two CDRs, can bind two antigens, and because of this feature is defined bivalent. The prototype antibody can be split by a variety of biological or chemical means. Each half of the antibody can only bind one antigen and, therefore, is defined monovalent. Means for preparing and characterizing antibodies are well known in the art (see, e.g., Howell and Lane, 1988).

Peptides corresponding to one or more antigenic determinants of a Chlamydia psittaci polypeptide of the present invention also can be prepared. Such peptides should generally be at least five or six amino acid residues in length, will preferably be about 10, 15, 20, 25 or about 30 amino acid residues in length, and may contain up to about 35-50 residues or so. Synthetic peptides will generally be about 35 residues long, which is the approximate upper length limit of automated peptide synthesis machines, such as those available from Applied Biosystems (Foster City, Calif.). Longer peptides also may be prepared, e.g., by recombinant means.

The identification and preparation of epitopes from primary amino acid sequences on the basis of hydrophilicity is taught in U.S. Pat. No. 4,554,101 (Hopp), incorporated herein by reference. Through the methods disclosed in Hopp, one of skill in the art would be able to identify epitopes from within an amino acid sequence such as a Chlamydia psittaci polypeptide sequence.

Numerous scientific publications have also been devoted to the prediction of secondary structure, and to the identification of epitopes, from analyses of amino acid sequences (Chou & Fasman, 1974a; Chou & Fasman, 1974b; Chou & Fasman, 1978a; Chou & Fasman, 1978b; Chou & Fasman, 1979). Any of these may be used, if desired, to supplement the teachings of Hopp in U.S. Pat. No. 4,554,101.

Moreover, computer programs are currently available to assist with predicting antigenic portions and epitopic core regions of proteins. Examples include those programs based upon the Jameson-Wolf analysis (Jameson & Wolf, 1988; Wolf et al., 1988), the program PEPPLOT® (Brutlag et al., 1990; Weinberger et al., 1985), and other new programs for protein tertiary structure prediction (Fetrow & Bryant, 1993). Another commercially available software program capable of carrying out such analyses is MACVECTOR (IBI, New Haven, Conn.).

In further embodiments, major antigenic determinants of a Chlamydia psittaci polypeptide may be identified by an empirical approach in which portions of the gene encoding the polypeptide are expressed in a recombinant host, and the resulting proteins tested for their ability to elicit an immune response. For example, PCR can be used to prepare a range of peptides lacking successively longer fragments of the C-terminus of the protein. The immunoactivity of each of these peptides is determined to identify those fragments or domains of the polypeptide that are immunodominant. Further studies in which only a small number of amino acids are removed at each iteration then allows the location of the antigenic determinants of the polypeptide to be more precisely determined.

Another method for determining the major antigenic determinants of a polypeptide is the SPOTS system (Genosys Biotechnologies, Inc., The Woodlands, Tex.). In this method, overlapping peptides are synthesized on a cellulose membrane, which following synthesis and deprotection, is screened using a polyclonal or monoclonal antibody. The antigenic determinants of the peptides which are initially identified can be further localized by performing subsequent syntheses of smaller peptides with larger overlaps, and by eventually replacing individual amino acids at each position along the immunoreactive peptide.

Once one or more such analyses are completed, polypeptides are prepared that contain at least the essential features of one or more antigenic determinants. The peptides are then employed in the generation of antisera against the polypeptide. Minigenes or gene fusions encoding these determinants also can be constructed and inserted into expression vectors by standard methods, for example, using PCR cloning methodology.

The use of such small peptides for antibody generation or vaccination typically requires conjugation of the peptide to an immunogenic carrier protein, such as hepatitis B surface antigen, keyhole limpet hemocyanin or bovine serum albumin. Methods for performing this conjugation are well known in the art.

1. Anti-Chlamydia psittaci Antibody Generation

The present invention provides monoclonal antibody compositions that are immunoreactive with a Chlamydia psittaci polypeptide. As detailed above, in addition to antibodies generated against a full length Chlamydia psittaci polypeptide, antibodies also may be generated in response to smaller constructs comprising epitopic core regions, including wild-type and mutant epitopes. In other embodiments of the invention, the use of anti-Chlamydia psittaci single chain antibodies, chimeric antibodies, diabodies and the like are contemplated.

As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting.

Monoclonal antibodies (mAbs) are recognized to have certain advantages, e.g., reproducibility and large-scale production, and their use is generally preferred.

However, “humanized” Chlamydia psittaci antibodies also are contemplated, as are chimeric antibodies from mouse, rat, goat or other species, fusion proteins, single chain antibodies, diabodies, bispecific antibodies, and other engineered antibodies and fragments thereof. As defined herein, a “humanized” antibody comprises constant regions from a human antibody gene and variable regions from a non-human antibody gene. A “chimeric antibody, comprises constant and variable regions from two genetically distinct individuals. An anti-Chlamydia psittaci humanized or chimeric antibody can be genetically engineered to comprise a Chlamydia psittaci antigen binding site of a given of molecular weight and biological lifetime, as long as the antibody retains its Chlamydia psittaci antigen binding site.

The term “antibody” is used to refer to any antibody-like molecule that has an antigen binding region, and includes antibody fragments such as Fab′, Fab, F(ab′)₂, single domain antibodies (DABs), Fv, scFv (single chain Fv), chimeras and the like. Methods and techniques of producing the above antibody-based constructs and fragments are well known in the art (U.S. Pat. No. 5,889,157; U.S. Pat. No. 5,821,333; U.S. Pat. No. 5,888,773, each specifically incorporated herein by reference).

U.S. Pat. No. 5,889,157 describes a humanized B3 scFv antibody preparation. The B3 scFv is encoded from a recombinant, fused DNA molecule, that comprises a DNA sequence encoding humanized Fv heavy and light chain regions of a B3 antibody and a DNA sequence that encodes an effector molecule. The effector molecule can be any agent having a particular biological activity which is to be directed to a particular target cell or molecule. Described in U.S. Pat. No. 5,888,773, is the preparation of scFv antibodies produced in eukaryotic cells, wherein the scFv antibodies are secreted from the eukaryotic cells into the cell culture medium and retain their biological activity. It is contemplated that similar methods for preparing multi-functional anti-Chlamydia psittaci fusion proteins, as described above, may be utilized in the present invention.

Means for preparing and characterizing antibodies also are well known in the art (See, e.g., Antibodies: A Laboratory Manual, Cold Spring Harbor Laboratory, 1988; incorporated herein by reference). The methods for generating monoclonal antibodies (mAbs) generally begin along the same lines as those for preparing polyclonal antibodies. Briefly, a polyclonal antibody is prepared by immunizing an animal with an immunogenic Chlamydia psittaci polypeptide composition in accordance with the present invention and collecting antisera from that immunized animal.

A wide range of animal species can be used for the production of antisera. Typically the animal used for production of antisera is a rabbit, a mouse, a rat, a hamster, a guinea pig or a goat. Because of the relatively large blood volume of rabbits, a rabbit is a preferred choice for production of polyclonal antibodies.

As is well known in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin also can be used as carriers. Means for conjugating a polypeptide to a carrier protein are well known in the art and include glutaraldehyde, m-maleimidobenzoyl-N-hydroxysuccinimide ester, carbodiimide and bis-biazotized benzidine.

As also well known in the art, the immunogenicity of a particular immunogen composition can be enhanced by the use of non-specific stimulators of the immune response, known as adjuvants. Suitable molecule adjuvants include all acceptable immunostimulatory compounds, such as cytokines, toxins or synthetic compositions.

Adjuvants that may be used include IL-1, IL-2, IL-4, IL-7, IL-12, γ-interferon, GMCSP, BCG, aluminum hydroxide, MDP compounds, such as thur-MDP and nor-MDP, CGP (MTP-PE), lipid A, and monophosphoryl lipid A (MPL). RIBI, which contains three components extracted from bacteria, MPL, trehalose dimycolate (TDM) and cell wall skeleton (CWS) in a 2% squalene/Tween 80 emulsion also is contemplated. MHC antigens may even be used. Exemplary, often preferred adjuvants include complete Freund's adjuvant (a non-specific stimulator of the immune response containing killed Mycobacterium tuberculosis), incomplete Freund's adjuvants and aluminum hydroxide adjuvant.

In addition to adjuvants, it may be desirable to coadminister biologic response modifiers (BRM), which have been shown to upregulate T cell immunity or downregulate suppressor cell activity. Such BRMs include, but are not limited to, Cimetidine (CIM; 1200 mg/d) (SmithKline Beecham, Pa.); low-dose Cyclophosphamide (CYP; 300 mg/m²) (Johnson/Mead, N.J.), cytokines such as γ-interferon, IL-2, or IL-12 or genes encoding proteins involved in immune helper functions, such as B-7.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization.

A second, booster injection, also may be given. The process of boosting and titering is repeated until a suitable titer is achieved. When a desired level of immunogenicity is obtained, the immunized animal can be bled and the serum isolated and stored, and/or the animal can be used to generate mAbs.

For production of rabbit polyclonal antibodies, the animal can be bled through an ear vein or alternatively by cardiac puncture. The removed blood is allowed to coagulate and then centrifuged to separate serum components from whole cells and blood clots. The serum may be used as is for various applications or else the desired antibody fraction may be purified by well-known methods, such as affinity chromatography using another antibody, a peptide bound to a solid matrix, or by using, e.g., protein A or protein G chromatography.

mAbs may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified Chlamydia psittaci polypeptide, peptide or domain, be it a wild-type or mutant composition. The immunizing composition is administered in a manner effective to stimulate antibody producing cells.

The methods for generating monoclonal antibodies (“Abs) generally begin along the same lines as those for preparing polyclonal antibodies. Rodents such as mice and rats are preferred animals, however, the use of rabbit, sheep or frog cells also is possible. The use of rats may provide certain advantages (Goding, 1986, pp. 60-61), but mice are preferred, with the BALB/c mouse being most preferred as this is most routinely used and generally gives a higher percentage of stable fusions.

The animals are injected with antigen, generally as described above. The antigen may be coupled to carrier molecules such as keyhole limpet hemocyanin if necessary. The antigen would typically be mixed with adjuvant, such as Freund's complete or incomplete adjuvant. Booster injections with the same antigen would occur at approximately two-week intervals, or the gene encoding the protein of interest can be directly injected.

Following immunization, somatic cells with the potential for producing antibodies, specifically B lymphocytes (B cells), are selected for use in the mAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible.

Often, a panel of animals will have been immunized and the spleen of an animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Any one of a number of myeloma cells may be used, as are known to those of skill in the art (Goding, pp. 65-66, 1986; Campbell, pp. 75-83, 1984). For example, where the immunized animal is a mouse, one may use P3-X63/Ag8, X63-Ag8.653, NS1/1.Ag 41, Sp210-Ag14, FO, NSO/IU, MPC-11, MPCll-X45-GTG 1.7 and S194/5XX0 Bul; for rats, one may use R210.RCY3, Y3-Ag 1.2.3, IR983F and 4B210; and U-266, GM1500-GRG2, LICR-LON-HMy2 and UC729-6 are all useful in connection with human cell fusions.

One preferred murine myeloma cell is the NS-1 myeloma cell line (also termed P3-NS-1-Ag4-1), which is readily available from the NIGMS Human Genetic Mutant Cell Repository by requesting cell line repository number GM3573. Another mouse myeloma cell line that may be used is the 8-azaguanine-resistant mouse murine myeloma SP2/0 non-producer cell line.

Methods for generating hybrids of antibody-producing spleen or lymph node cells and myeloma cells usually comprise mixing somatic cells with myeloma cells in a 2:1 proportion, though the proportion may vary from about 20:1 to about 1:1, respectively, in the presence of an agent or agents (chemical or electrical) that promote the fusion of cell membranes. Fusion methods using Sendai virus have been described by Kohler and Milstein (1975; 1976), and those using polyethylene glycol (PEG), such as 37% (v/v) PEG, by Gefter et al. (1977). The use of electrically induced fusion methods also is appropriate (Goding pp. 71-74, 1986).

Fusion procedures usually produce viable hybrids at low frequencies, about 1×10⁻⁶ to 1×10⁻⁸. However, this does not pose a problem, as the viable, fused hybrids are differentiated from the parental, unfused cells (particularly the unfused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. HAT medium, a growth medium containing hypoxanthine, aminopterin and thymidine, is well known in the art as a medium for selection of hybrid cells. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

The preferred selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells.

This culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays, dot immunobinding assays, and the like.

The selected hybridomas then would be serially diluted and cloned into individual antibody-producing cell lines, which clones can then be propagated indefinitely to provide mAbs. The cell lines may be exploited for mAb production in two basic ways. First, a sample of the hybridoma can be injected (often into the peritoneal cavity) into a histocompatible animal of the type that was used to provide the somatic and myeloma cells for the original fusion (e.g., a syngeneic mouse). Optionally, the animals are primed with a hydrocarbon, especially oils such as pristane (tetramethylpentadecane) prior to injection. The injected animal develops tumors secreting the specific monoclonal antibody produced by the fused cell hybrid. The body fluids of the animal, such as serum or ascites fluid, can then be tapped to provide mAbs in high concentration. Second, the individual cell lines could be cultured in vitro, where the mAbs are naturally secreted into the culture medium from which they can be readily obtained in high concentrations.

mAbs produced by either means may be further purified, if desired, using filtration, centrifugation and various chromatographic methods such as HPLC or affinity chromatography. Fragments of the monoclonal antibodies of the invention can be obtained from the monoclonal antibodies so produced by methods which include digestion with enzymes, such as pepsin or papain, and/or by cleavage of disulfide bonds by chemical reduction. Alternatively, monoclonal antibody fragments encompassed by the present invention can be synthesized using an automated peptide synthesizer.

It also is contemplated that a molecular cloning approach may be used to generate monoclonals. For this, combinatorial immunoglobulin phagemid libraries are prepared from RNA isolated from the spleen of the immunized animal, and phagemids expressing appropriate antibodies are selected by panning using cells expressing the antigen and control cells. The advantages of this approach over conventional hybridoma techniques are that approximately 104 times as many antibodies can be produced and screened in a single round, and that new specificities are generated by H and L chain combination which further increases the chance of finding appropriate antibodies.

Alternatively, monoclonal antibody fragments encompassed by the present invention can be synthesized using an automated peptide synthesizer, or by expression of full-length gene or of gene fragments in, for example, E. coli.

F. Pharmaceutical Compositions

Aqueous compositions of the present invention comprise an effective amount of a purified polynucleotide comprising a Chlamydia psittaci sequence and/or a purified a protein, polypeptide, peptide, epitopic core region of a Chlamydia psittaci protein, and the like, dissolved and/or dispersed in a pharmaceutically acceptable carrier and/or aqueous medium. Aqueous compositions of gene therapy vectors expressing any of the foregoing are also contemplated.

The phrases “pharmaceutically and/or pharmacologically acceptable” refer to molecular entities and/or compositions that do not produce an adverse, allergic and/or other untoward reaction when administered to an animal.

As used herein, “pharmaceutically acceptable carrier” includes any and/or all solvents, dispersion media, coatings, antibacterial and/or antifungal agents, isotonic and/or absorption delaying agents and the like. The use of such media and/or agents for pharmaceutical active substances is well known in the art. Except insofar as any conventional media and/or agent is incompatible with the active ingredient, its use in the therapeutic compositions is contemplated. Supplementary active ingredients can also be incorporated into the compositions. For animal and more particularly human administration, preparations should meet sterility, pyrogenicity, general safety and/or purity standards as required by FDA Office of Biologics standards.

The biological material should be extensively dialyzed to remove undesired small molecular weight molecules and/or lyophilized for more ready formulation into a desired vehicle, where appropriate. The active compounds may generally be formulated for parenteral administration, e.g., formulated for injection via the intravenous, intramuscular, sub-cutaneous, intralesional, and/or even intraperitoneal routes, or formulated for oral or inhaled delivery. The preparation of an aqueous compositions that contain an effective amount of purified Chlamydia psittaci polynucleotide or polypeptide agent as an active component and/or ingredient will be known to those of skill in the art in light of the present disclosure. Typically, such compositions can be prepared as injectables, either as liquid solutions and/or suspensions; solid forms suitable for using to prepare solutions and/or suspensions upon the addition of a liquid prior to injection can also be prepared; and/or the preparations can also be emulsified.

The pharmaceutical forms suitable for injectable use include sterile aqueous solutions and/or dispersions; formulations including sesame oil, peanut oil and/or aqueous propylene glycol; and/or sterile powders for the extemporaneous preparation of sterile injectable solutions and/or dispersions. In all cases the form must be sterile and/or must be fluid to the extent that easy syringability exists. It must be stable under the conditions of manufacture and/or storage and/or must be preserved against the contaminating action of microorganisms, such as bacteria and/or fungi.

Solutions of the active compounds as free base and/or pharmacologically acceptable salts can be prepared in water suitably mixed with a surfactant, such as hydroxypropylcellulose. Dispersions can also be prepared in glycerol, liquid polyethylene glycols, and/or mixtures thereof and/or in oils. Under ordinary conditions of storage and/or use, these preparations contain a preservative to prevent the growth of microorganisms.

A Chlamydia psittaci polynucleotide or polypeptide of the present invention can be formulated into a composition in a neutral and/or salt form. Pharmaceutically acceptable salts, include the acid addition salts (formed with the free amino groups of the protein) and/or which are formed with inorganic acids such as, for example, hydrochloric and/or phosphoric acids, and/or such organic acids as acetic, oxalic, tartaric, mandelic, and/or the like. Salts formed with the free carboxyl groups can also be derived from inorganic bases such as, for example, sodium, potassium, ammonium, calcium, and/or ferric hydroxides, and/or such organic bases as isopropylamine, trimethylamine, histidine, procaine and/or the like. In terms of using peptide therapeutics as active ingredients, the technology of U.S. Pat. Nos. 4,608,251; 4,601,903; 4,599,231; 4,599,230; 4,596,792; and/or 4,578,770, each incorporated herein by reference, may be used.

The carrier can also be a solvent and/or dispersion medium containing, for example, water, ethanol, polyol (for example, glycerol, propylene glycol, and/or liquid polyethylene glycol, and/or the like), suitable mixtures thereof, and/or vegetable oils. The proper fluidity can be maintained, for example, by the use of a coating, such as lecithin, by the maintenance of the required particle size in the case of dispersion and/or by the use of surfactants. The prevention of the action of microorganisms can be brought about by various antibacterial and/or antifungal agents, for example, parabens, chlorobutanol, phenol, sorbic acid, thimerosal, and/or the like. In many cases, it will be preferable to include isotonic agents, for example, sugars and/or sodium chloride. Prolonged absorption of the injectable compositions can be brought about by the use in the compositions of agents delaying absorption, for example, aluminum monostearate and/or gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the required other ingredients from those enumerated above. In the case of sterile powders for the preparation of sterile injectable solutions, the preferred methods of preparation are vacuum-drying and/or freeze-drying techniques which yield a powder of the active ingredient plus any additional desired ingredient from a previously sterile-filtered solution thereof. The preparation of more, and/or highly, concentrated solutions for direct injection is also contemplated, where the use of DMSO as solvent is envisioned to result in extremely rapid penetration, delivering high concentrations of the active agents to a small tumor area.

Upon formulation, solutions will be administered in a manner compatible with the dosage formulation and/or in such amount as is therapeutically effective. The formulations are easily administered in a variety of dosage forms, such as the type of injectable solutions described above, but drug release capsules and/or the like can also be employed.

For parenteral administration in an aqueous solution, for example, the solution should be suitably buffered if necessary and/or the liquid diluent first rendered isotonic with sufficient saline and/or glucose. These particular aqueous solutions are especially suitable for intravenous, intramuscular, subcutaneous and/or intraperitoneal administration. In this connection, sterile aqueous media which can be employed will be known to those of skill in the art in light of the present disclosure. For example, one dosage could be dissolved in 1 ml of isotonic NaCl solution and/or either added to 1000 ml of hypodermoclysis fluid and/or injected at the proposed site of infusion, (see for example, “Remington's Pharmaceutical Sciences” 15th Edition, pages 1035-1038 and/or 1570-1580). Some variation in dosage will necessarily occur depending on the condition of the subject being treated. The person responsible for administration will, in any event, determine the appropriate dose for the individual subject.

A Chlamydia psittaci polynucleotide or protein-derived peptides and/or agents may be formulated within a therapeutic mixture to comprise about 0.0001 to 1.0 milligrams, and/or about 0.001 to 0.1 milligrams, and/or about 0.1 to 1.0 and/or even about 10 milligrams per dose and/or so. Multiple doses can also be administered.

In addition to the compounds formulated for parenteral administration, such as intravenous and/or intramuscular injection, other pharmaceutically acceptable forms include, e.g., tablets and/or other solids for oral administration; liposomal formulations; time release capsules; and/or any other form currently used, including cremes.

One may also use nasal solutions and/or sprays, aerosols and/or inhalants in the present invention. Nasal solutions are usually aqueous solutions designed to be administered to the nasal passages in drops and/or sprays. Nasal solutions are prepared so that they are similar in many respects to nasal secretions, so that normal ciliary action is maintained. Thus, the aqueous nasal solutions usually are isotonic and/or slightly buffered to maintain a pH of 5.5 to 6.5. In addition, antimicrobial preservatives, similar to those used in ophthalmic preparations, and/or appropriate drug stabilizers, if required, may be included in the formulation. Various commercial nasal preparations are known and/or include, for example, antibiotics and/or antihistamines and/or are used for asthma prophylaxis.

Additional formulations which are suitable for other modes of administration include vaginal suppositories and/or pessaries. A rectal pessary and/or suppository may also be used. Suppositories are solid dosage forms of various weights and/or shapes, usually medicated, for insertion into the rectum, vagina and/or the urethra. After insertion, suppositories soften, melt and/or dissolve in the cavity fluids. In general, for suppositories, traditional binders and/or carriers may include, for example, polyalkylene glycols and/or triglycerides; such suppositories may be formed from mixtures containing the active ingredient in the range of 0.5% to 10%, preferably 1%-2%.

Oral formulations include such normally employed excipients as, for example, pharmaceutical grades of mannitol, lactose, starch, magnesium stearate, sodium saccharine, cellulose, magnesium carbonate and/or the like. These compositions take the form of solutions, suspensions, tablets, pills, capsules, sustained release formulations and/or powders. In certain defined embodiments, oral pharmaceutical compositions will comprise an inert diluent and/or assimilable edible carrier, and/or they may be enclosed in hard and/or soft shell gelatin capsule, and/or they may be compressed into tablets, and/or they may be incorporated directly with the food of the diet. For oral therapeutic administration, the active compounds may be incorporated with excipients and/or used in the form of ingestible tablets, buccal tables, troches, capsules, elixirs, suspensions, syrups, wafers, and/or the like. Such compositions and/or preparations should contain at least 0.1% of active compound. The percentage of the compositions and/or preparations may, of course, be varied and/or may conveniently be between about 2 to about 75% of the weight of the unit, and/or preferably between 25-60%. The amount of active compounds in such therapeutically useful compositions is such that a suitable dosage will be obtained.

The tablets, troches, pills, capsules and/or the like may also contain the following: a binder, as gum tragacanth, acacia, cornstarch, and/or gelatin; excipients, such as dicalcium phosphate; a disintegrating agent, such as corn starch, potato starch, alginic acid and/or the like; a lubricant, such as magnesium stearate; and/or a sweetening agent, such as sucrose, lactose and/or saccharin may be added and/or a flavoring agent, such as peppermint, oil of wintergreen, and/or cherry flavoring. When the dosage unit form is a capsule, it may contain, in addition to materials of the above type, a liquid carrier. Various other materials may be present as coatings and/or to otherwise modify the physical form of the dosage unit. For instance, tablets, pills, and/or capsules may be coated with shellac, sugar and/or both. A syrup of elixir may contain the active compounds sucrose as a sweetening agent methyl and/or propylparabens as preservatives, a dye and/or flavoring, such as cherry and/or orange flavor.

G. Kits

Therapeutic kits of the present invention are kits comprising a Chlamydia psittaci polynucleotide or polypeptide. Such kits will generally contain, in suitable container means, a pharmaceutically acceptable formulation of a Chlamydia psittaci polynucleotide or polypeptide or vector expressing any of the foregoing in a pharmaceutically acceptable formulation. The kit may have a single container means, and/or it may have distinct container means for each compound.

When the components of the kit are provided in one and/or more liquid solutions, the liquid solution is an aqueous solution, with a sterile aqueous solution being particularly preferred. The Chlamydia psittaci polynucleotide or polypeptide compositions may also be formulated into a syringeable composition. In which case, the container means may itself be a syringe, pipette, and/or other such like apparatus, from which the formulation may be applied to an infected area of the body, injected into an animal, and/or even applied to and/or mixed with the other components of the kit.

However, the components of the kit may be provided as dried powder(s). When reagents and/or components are provided as a dry powder, the powder can be reconstituted by the addition of a suitable solvent. It is envisioned that the solvent may also be provided in another container means.

The container means will generally include at least one vial, test tube, flask, bottle, syringe and/or other container means, into which the Chlamydia psittaci polynucleotide or polypeptide formulation are placed, preferably, suitably allocated. The kits may also comprise a second container means for containing a sterile, pharmaceutically acceptable buffer and/or other diluent.

The kits of the present invention will also typically include a means for containing the vials in close confinement for commercial sale, such as, e.g., injection and/or blow-molded plastic containers into which the desired vials are retained.

Irrespective of the number and/or type of containers, the kits of the invention may also comprise, and/or be packaged with, an instrument for assisting with the injection/administration and/or placement of the ultimate Chlamydia psittaci polynucleotide or polypeptide within the body of an animal. Such an instrument may be a syringe, pipette, forceps, and/or any such medically approved delivery vehicle.

### H. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Exemplary ELI Protocol

The following sections outline general methodology that one might use to prepare, screen and utilize ELI according to the present invention. Of course the following methods are merely general guidelines and should not limit one of skill in the art from modifying the present invention to accomplish a desired goal using ELI.

1. Library Construction

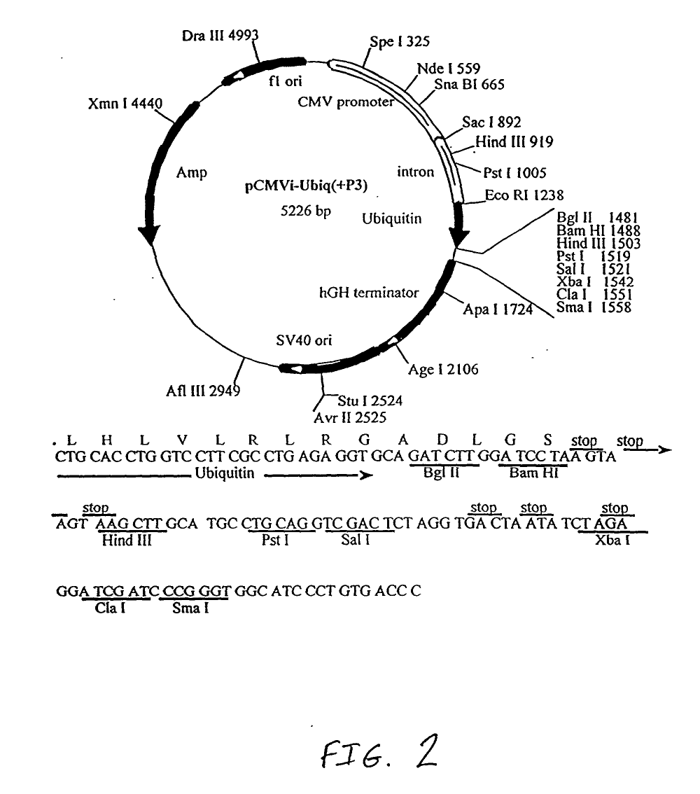

The present invention provides expression library constructs of genus Chlamydia psittaci. An expression library of C. psittaci can be produced by first physically shearing the genomic DNA of C. psittaci (e.g., C. psittaci strain B577) and size-selecting fragments of 300-800 base pairs. The protocol used by the present inventors to produce a C. psittaci library is similar to that described in Sykes and Johnston (1999). Adaptors were added and the DNA fragments ligated into a genetic immunization vector (FIG. 2) designed to link fragments to the mouse ubiquitin gene. However, the fragments can be blunt-end cloned.

This vector is known to enhance MHC class I-restricted immune responses (Sykes and Johnston, 1999), while sterilizing immunity against Chlamydia is thought to be MHC class II-dependent (Morrison et al., 1995). However, any genetic immunization procedure, by the mechanism of intracellular expression of the inserted genes, will target towards class I antigen presentation. Nevertheless, both MHC class I- and class II-restricted immune responses to the expressed antigens are well documented (Barry et al., 1995; Sykes and Johnston, 1999). The inventors observed, for instance, pronounced delayed-type hypersensitivity responses, mediated by MHC II-restricted CD4⁺ Th1 cells, against protective C. psittaci B577 antigens, which were expressed from the ubiquitin fusion vector. In addition to the fact that MHC II-restricted immunity is generated by the ubiquitin fusion vector, MHC I-restricted immunity appears to mediate protection in the early phase of chlamydial infection (Morrison et al., 1995; Rottenberg et al., 1999). This duality of the cellular immune response generated by the ubiquitin fusion vector might explain the efficacy of this vector for genetic immunization against intracellular bacteria.

A library of approximately 82,000 individual members was created and tested as 27 sub-libraries each with 2,400-3,400 plasmid clones. The average insert frequency was approximately 67% and the average insert size was 660 base pairs. Nitrocellulose replica filters were made of each original colony plating of a sub-library pool for subsequent retrieval of positive clones. This generated a library with approximately six-fold expression-equivalent redundancy. One expression equivalent is defined as the number of in-frame fragments necessary to completely represent all authentic open reading frames. Since the genome size of C. psittaci is approximately 1×10⁶base pairs and only one-sixth of the actual open-reading frames will be cloned in the right orientation and frame, it requires at least six genomic equivalents to encode one expression equivalent. Each sub-library was propagated on plates and harvested to prepare DNA. DNA representing each sub-library was used for genetic immunization of mice in the following section.

2. Vaccination and Challenge

For the first round of testing, outbred, 6-week old, female N1H-Swiss Webster mice were inoculated with the purified DNA of each sub-library using both intra-muscular (i.m.) and epidermal injection. The epidermal injection was effected with a gene gun (Sanford et al., 1991). Each mouse was given 50 μg DNA i.m. and 5 μg DNA by gene gun. It has been argued that the gene gun immunization favors a Th2 and the i.m. injection a Th1 type response (Feltquate et al., 1997), therefore both types of injection were given to each group. In the first round of testing, the prime inoculation was followed by a boost 9 weeks later, before intranasal challenge with 3×10⁶ inclusion forming units (IFU) of C. psittaci strain B577 13 weeks after prime inoculation. The animals were sacrificed 12 days after the challenge, and lungs were weighed.

3. Library Deconvolution

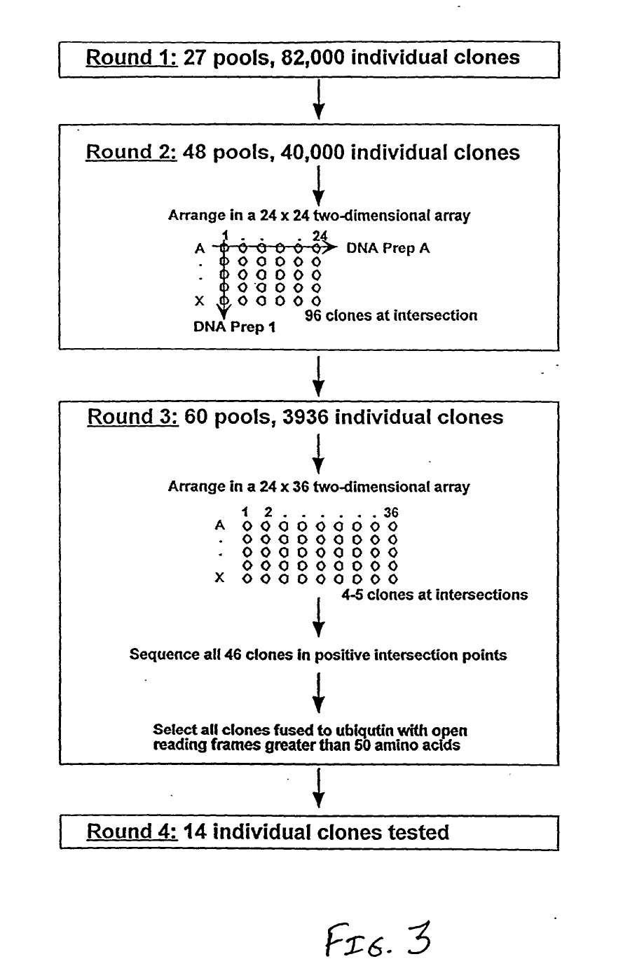

The basic scheme for handling the reduction of the libraries is depicted in FIG. 3. Fourteen groups out of the first round looked promising, so the individual clones from these groups were picked and grown in 96 well microtiter plates. This gave approximately 40,000 wells in microtiter plates, therefore about 40,000 clones. The second round was reduced using a two dimensional array format. As depicted in FIG. 3, the DNA was prepared from colonies pooled from rows and columns of the array. The rationale was that if a row and column conferred protection, the colonies at the intersection would be responsible. This scheme is premised on largely additive effects of the protective clones. This 24×24 array yielded pools of 1,700 clones with each intercession having ˜96 clones. Currently the inventors deconvolute the second round with a 3-dimesional array.

Since the lung weight was highly variable in the outbred N1H-Swiss mice with variable MHC background, the inventors decided to use inbred BALB/c mice in subsequent rounds. The 48 DNA pools for round two were i.m. injected into BALB/c mice at 50 μg DNA/animal, and the animals were boosted at seven weeks by both gene gun inoculation and i.m. injection. The mice were given a higher C. psittaci challenge, 1.6×10⁶ IFU C. psittaci B577, at approximately 12 weeks, again to further differentiate the groups. Animals were sacrificed and results evaluated as in round one.

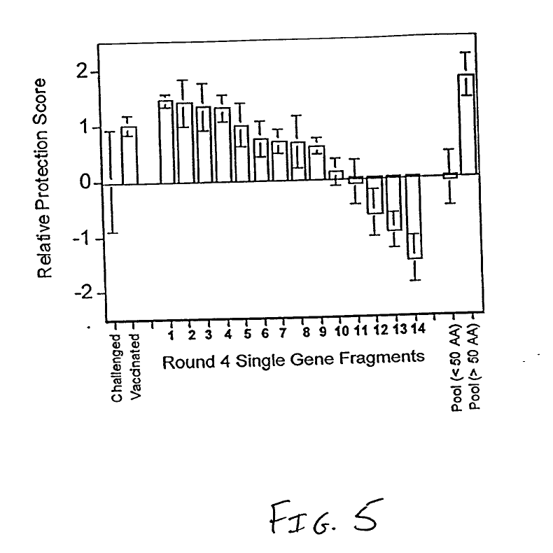

In the fourth round, the animals received two boosts rather than one, and the challenge inoculum was increased to 3×10⁶ IFU C. psittaci B577 to increase the selectivity of protection scoring. Furthermore, because too much DNA may lead to a decrease in cellular immune response, the amount of each individual clone was reduced by half, with the difference made up with pUC 118 DNA, so each mouse received a total 50 μg DNA for i.m. immunization, but only 25 μg of the specific clone. The inventors also decreased the gene gun DNA in the same manner: 1.25 μg/ear of the specific clone and 1.25 μg pUC118. Mice were boosted i.m. at both four and nine weeks after prime inoculation, and were challenged. The results of this final round are depicted in FIG. 5.

4. Analysis of Sequences

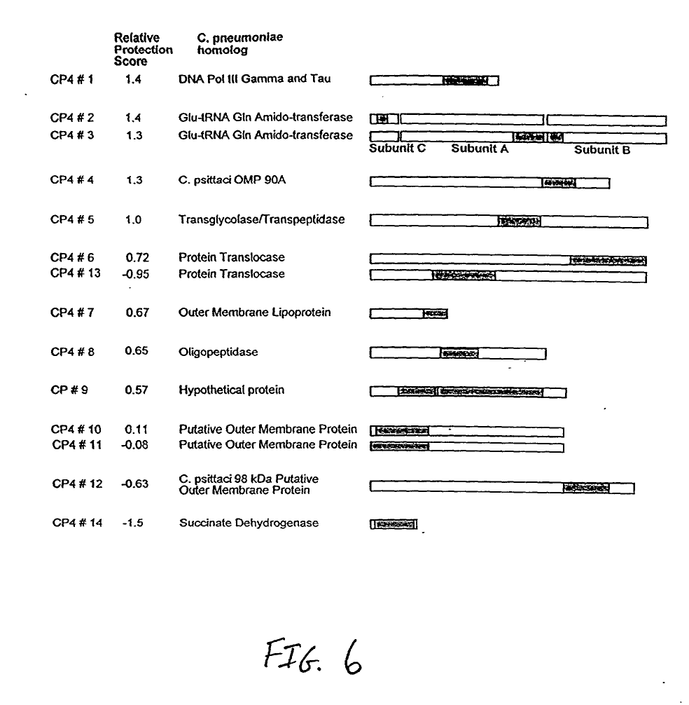

The clones conferring protection were re-sequenced and then compared by BLAST search to Genbank and particularly to the recently completed C. pneumoniae (Kalman et al., 1999) genome sequences (FIG. 6). Of the 14 single genes identified in this study, ten are internal fragments and three contain the C-terminus of the protein. Of the five most protective clones, one was from a putative outer membrane protein and one was from a cell surface protein. The other three were from cytosolic proteins.

Four of the 14 clones have sequence similarity to a class of proteins known as putative outer membrane proteins (POMPs) in Chlamydia psittaci and Chlamydia pneumoniae. Many of the “putative” outer membrane proteins are known to be localized to the outer membrane and to be highly immunogenic (Longbottom et al., 1996; Tan et al., 1990).

5. Mixing Experiment

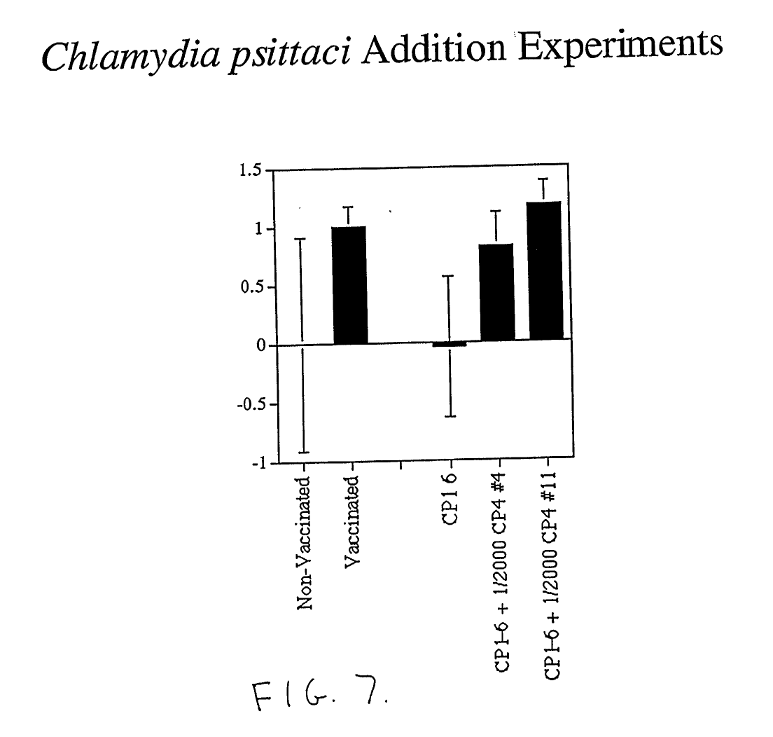

The two dimensional approach used to find protective gene fragments assumes that the protection is due to a single highly protective gene within a pool. To verify that such genes would be found, 25 ng (i.e. 1/2000) of either of the two most protective genes was added to a pool that scored negative (pool 6 round 1). As depicted in FIG. 7, spiking with either clone converted the negative library to a positive.

### Example 2

### Materials and Methods

Library construction. C. psittaci strain B577 (ATCC VR-656) was grown in BGMK cells and elementary bodies (EB) were purified by renograff gradient centrifugation as described (Huang et al., 1999). Genomic DNA was isolated from EB by proteinase K and RNase digestion followed by cetyl-trimethyl ammonium bromide (Kaltenbock et al., 1997).

Genomic DNA was physically sheared using a nebulizer (Glas Col, Terra Haute, Ind.), then size fractioned on a 1.5% TBE agarose gel. Agarose with fragments between 300-700 base pairs was excised and the DNA was electroeluted. Adaptors (top strand 5′: GATCTGGATCCCGAT (SEQ ID NO.2) ATCGGGCTCCA (SEQ ID NO.3) onto the fragments, then the fragments were cloned into pCMVi-UBs at the Bgl II site (See FIG. 6 and Sykes and Johnston, 1999 for more details). The ligations were transformed into DH alpha electrocompetent cells and plated onto 150 mm diameter YT-Ampicillin (75 μg/mL final concentration) plates. The resulting plates had between 2400-3400 individual clones per plate. After the plates were incubated overnight at 37° C., the colonies from were lifted using nitrocellulose filters soaked in L-Broth with 8% DMSO, and these filters were stored at −80° C. The original agar plates were then incubated at 37° C. for an additional six hours. Ten mL of L Broth was added to each plate, the E. coli was scraped into 150 mL of L-Broth and grown at 37° C. for 30 minutes. Ampicillin was then added to a final concentration of 50 μg/mL, and the cultures were grown overnight at 37° C. Cells were pelleted and the DNA was purified using Qiagen tip 500 columns.

Inoculation of DNA. Round One: DNA from the pools was injected into 6-week old female N1H-Swiss mice. All mice received 50 μg total DNA by i.m. injections, evenly distributed between the quadriceps and tibialis anterior muscles. Eighteen of the groups also received gene gun inoculations (wand), with 2.5 μg DNA inoculated into each ear. The animals were boosted once at nine weeks in the same manner as the primary inoculation—all mice received i.m. injections, but only the same 18 groups received gene gun injections—then intranasally challenged with 5.5×10⁵ IFU of C. psittaci strain B577 at 13 weeks. The mice were sacrificed 11 days after the challenge, and lungs were weighed.

Round Two: Nitrocellulose filters from the positive pools were placed on L-Broth Bio-Assay plates supplemented with 75 μg/mL ampicillin and 2% agar. The filters were incubated on the plates for approximately 15 minutes, then the nitrocellulose was discarded. The colonies were grown at 30° C. for 12 hours. The majority of the colonies were picked into 96 well microtiter plates containing HYT media (1.6% Bacto-tryptone, 1.0% Bacto-yeast extract, 85.5 mM NaCl, 36 mM K₂HPO₄, 13.2 mM KH₂PO₄, 1.7 mM Sodium citrate, 0.4 mM MgSO4, 6.8 mM ammonium sulfate, 4.4% wt/vol glycerol) supplemented with 75 μg/mL ampicillin, using a Hybaid colony picker; the plates were then visually inspected and the remainder of the colonies were hand-picked. The microtiter plates were designated by their original pool number and by the order in which they were picked. Hence, plate 5.10 was from original pool 5 and was the tenth plate picked. The colonies were subdivided into groups as is indicated in FIG. 2. All of the microtiter plates comprising a pool were stamped onto on L-Broth Bio-Assay plates supplemented with 75 μg/mL ampicillin and were grown overnight at 37° C. The cells from these plates were harvested by adding L-Broth to the plates and scraping off the cells. The cells were pelleted by centrifugation then resuspended in Qiagen buffer P1. The remainder of the DNA prep proceeded according to manufacture's instructions.

These 48 DNA pools were i.m. injected into 6-week old BALB/c mice at 50 μg DNA/animal. For the initial inoculation, the mice did not receive gene gun inoculations. At seven weeks, the mice were boosted with 50 μg DNA/animal. In addition to the i.m. injections, the first 31 groups received gene gun (Rumsey-Loomis) inoculations at 2.5 μg DNA/ear; however, the gene gun failed at group 32, and the last 17 groups received only i.m. injections. The mice were given a higher challenge, 1.6×10⁶ IFU C. psittaci B577, at 12 weeks. Animals were sacrificed as in round one.

Round Three: Colonies from the microtiter plates that were judged to be positive were arrayed as in FIG. 2. For each pool, new microtiter plates with HYT media supplemented with 75 μg/mL ampicillin were constructed from all of the colonies which comprise the. Colonies were grown and DNA prepared as in round two.

The mice received both gene gun (wand) and i.m. inoculations at the dosage indicated above. At six weeks, the mice were boosted with 50 μg DNA/animal, but only by i.m. injections. The challenge schedule was the same as in Round Two.

Round Four: E. coli from wells at either full by full protection or full by partial protection was streaked out onto YT-plates supplemented with 75 μg/mL ampicillin. Six colonies from each of the plates were tested by PCR colony screening, using the primers FS-UB 5′: CCGCACCCTCTCTGATTAC (SEQ ID No: 4) CTGGAGTGGCAACTTCC. (SEQ ID NO. 5) Colonies with different sizes, hence different inserts, were sequenced using ABI Big Dye terminator and the FS-UB primer. Samples were purified on G-50 spin columns, and run on an ABI 377 Sequencer. The generated sequences were analyzed for open reading frames using a program designed by Simon Raynor, Ph.D.

### Example 3

### Vaccination and Challenge

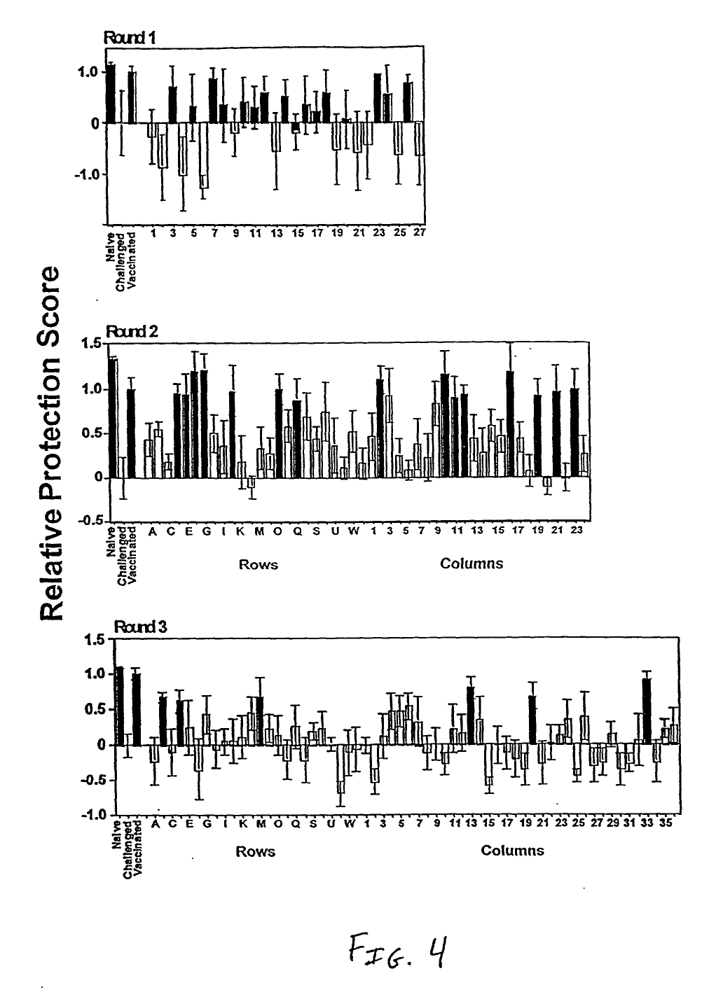

It was established that the weight increase of the infected lung over the lung weight of naive, uninfected controls (˜120 mg) correlated strongly with disease intensity. Maximum disease in this model resulted in approximately 250% lung weight increase, while further lung weight increases were lethal. The lung disease on day 12 after inoculation was characterized by areas of gross lung tissue consolidation and the presence of mononuclear interstitial infiltrates in consolidated tissue. Chlamydial inclusions were observed by immunohistochemistry in many macrophages, but rarely in other cells. Controls for complete protection were established by low level intranasal infection of naïve mice with 3×10⁴ IFU of C. psittaci strain B5774 weeks prior to challenge. These mice were completely protected from disease after challenge infection and had lung weight increases of 10-30% compared to naive animals. Lungs of completely protected mice did not show gross lung lesions, and pathohistological examination revealed no interstitial infiltrates, but prominent peribronchiolar lymphocytic cuffs, interpreted as sign of protective immune stimulation. The chlamydial lung burden on day 11 after challenge was typically 1-3×10⁶ IFU per 100 mg lung tissue in protected, and 2-6×10⁶ IFU per 100 mg lung in diseased animals. Since the lowest chlamydial burden was, however, not consistently associated with lowest disease, the inventors used the disease-dependent parameter lung weight rather than chlamydial burden as readout for evaluation of protection. The lung weights were transformed to relative protection scores in a linear equation that assumed the high average lung weight of the severely ill, naïve, challenged mice as 0 and that of fully protected controls as 1 (FIG. 4).

### Example 4

### Deconvolution of the Libraries

Since the lung weight was highly variable in the outbred N1H-Swiss mice with variable MHC background, the inventors decided to use inbred BALB/c mice in subsequent rounds. The 48 DNA pools for round two were i.m. injected into BALB/c mice at 50 μg DNA/animal, and the animals were boosted at seven weeks by both gene gun inoculation and i.m. injection. The mice were given a higher C. psittaci challenge, 1.6×10⁶ IFU C. psittaci B577, at approximately 12 weeks, again to further differentiate the groups. Animals were sacrificed and results evaluated as in round one.

The results of the Round two challenge are depicted in FIG. 4. Of the 48 groups from round two, 15 were judged to be positive, giving a total of 3936 wells. These wells were again arrayed as in round two, but the array had 112 colonies per column and 156 per row with 4-5 colonies per intersection (See FIG. 3). The mice received both gene gun and i.m. injections at the dosage indicated above. At six weeks, the mice were boosted. Both the challenge and the sacrifice were performed as in Round two.

The positive 46 colonies from the intersection wells from Round three were sequenced, and those clones with open reading frames greater than 50 amino acids long were prepared individually and shot into mice as single genes and as a pool. Fourteen clones met these criteria. The disease scoring on each pool in rounds 1-3 are depicted in FIG. 4.

In the fourth round, the animals received two boosts rather than one, and the challenge inoculum was increased to 3×10⁶ IFU C. psittaci B577 to increase the selectivity of protection scoring. Furthermore, because too much DNA may lead to a decrease in cellular immune response, the amount of each individual clone was reduced by half but made up the difference with pUC 118 DNA, and each mouse received a total 50 μg DNA for i.m. immunization, but only 25 μg of the specific clone. The inventors also decreased the gene gun DNA in the same manner: 1.25 μg/ear of the specific clone and 1.25 μg pUCI18. Mice were boosted i.m. at both four and nine weeks after prime inoculation, and were challenged. The results of this final round are depicted in FIG. 5.

### Example 5

### Comparison of Clones

Based on the hypothesis that sequences from genes conferring a high level of protection might be selected more than once in the ELI process, the clones were compared against each other for overlaps. Interestingly, one of the clones, CP4 #10, did overlap with another gene, CP4 #11. The gene from which these two clones arise had been partially sequenced (Longbottom et al., 1998).

Two of the genes, CP4 #5 and CP4 #9, had an overlapping region, but they were fused to ubiqutin in opposite orientations. CP4 #5, is composed of two different C. psittaci DNA fragments, fused in opposite orientations. The first gene is fused to ubiqutin in the correct orientation and the correct reading frame. Interestingly, the second gene, which is in the opposite orientation to the ubiqutin gene, has an overlapping sequence to CP4 #5. It is doubtful that the protein from the second gene is produced in the mouse.

### Example 6

### Analysis of Sequences

The clones conferring protection were re-sequenced and then compared by BLAST search to Genbank and particularly to the recently completed C. pneumoniae (Kalman et al., 1999) genome sequences (FIG. 6). The full-length Chlamydia psittaci genes were next isolated and sequences. Upon analysis, all nucleic acid sequences, except #4, #10, #11, and #12, were previously undisclosed in any context. Further, only protions of the sequences encoding #10 and #11 were previously disclosed.

Since most protective genes would not have been predicted by any bioinformatics or information-based approach, it is likely that one will need to apply an unbiased, global approach, such as ELI to define vaccine candidates.

Table 2, lists a comparision of the Chlamydia psittaci genes with homologues from Chlamydia trachomatis and Chlamydia pneumoniae.

TABLE 2

| | C. psittaci | C. trachomatis | Identity/Similarity | C. pneumoniae | Identity/Similarity |
|---|---|---|---|---|---|
| CP4 #1 | | DNA Pol III Gamma and Tau | 62/73 | DNA Pol III Gamma and Tau | 66/76 |
| CP4 #2 | | Glu-tRNA Gln Amido- | 49/70 | Glu-tRNA Gln Amido- | 48/63 |
| | | transferase (C subunit) | | transferase (C subunit) | |
| CP4 #3 | | Glu-tRNA Gln Amido- | 71/85 | Glu-tRNA Gln Amido- | 71/84 |
| | | transferase (A subunit) | | transferase (A subunit) | |
| CP4 #4 | OMP 90A | Outer Membrane Protein 5 | 30/45 | Outer Membrane Protein I | 40/54 |
| CP4 #5 | | Transglycolase/transpeptidase | 67/80 | Transglycolase/transpeptidase | 67/77 |
| CP4 #6 | | Protein Translocase | 80/89 | Protein Translocase | 84/92 |
| CP4 #7 | | | | Outer Membrane Lipoprotein | 60/79 |
| CP4 #8 | | Oligopeptidase | 60/75 | Oligopeptidase | 61/74 |
| CP4 #9 | | Hypothetical protein | 62/76 | Hypothetical protein | 62/77 |
| CP4 #10 | | Outer Membrane Protein 4 | 27/42 | Outer Membrane Protein G | 33/51 |
| CP4 #11 | | Outer Membrane Protein 4 | 27/42 | Outer Membrane Protein G | 33/51 |
| CP4 #12 | OMP 98 kDa | Outer Membrane Protein 5 | 30/43 | Outer membrane Protein G | 44/58 |
| CP4 #13 | | Protein Translocase | 80/89 | Protein Translocase | 84/92 |
| CP4 #14 | | Succinate Dehydrogenase | 60/76 | Succinate Dehydrogenase | 61/77 |

Table 3 lists all of the cloned fragments, their corresponsing full length nucleotide sequences, and the amino acid sequences encoded by both the fragments and the full length sequences. Table 2 further describes the fragments.

TABLE 3
SEQUENCE LISTING INDEX

| SEQ ID NO | CP4 NO | Description |
|---|---|---|
| SEQ ID NO: 6 | CP4 #1 | (fragment) homolog to C. pneumoniae DNA Pol III Gamma |
| | | and Tau subunits (dnaX2 gene) |
| SEQ ID NO: 7 | CP4 #1 | Polypeptide translation corresponding to SEQ ID |
| | | NO. 6, homolog to C. pneumoniae DNA Pol III |
| | | Gamma and Tau subunits (dnaX2 gene) |
| SEQ ID NO: 8 | CP4 #1 | (full length) homolog to C. pneumoniae DNA Pol |
| | | III Gamma and Tau subunits (dnaX2 gene) |
| SEQ ID NO: 9 | CP4 #1 | Polypeptide translation corresponding to SEQ ID |
| | | NO. 8, homolog to C. pneumoniae DNA Pol III |
| | | Gamma and Tau subunits (dnaX2 gene) |
| SEQ ID NO: 10 | CP4 #2 | (fragment) homolog to C. pneumoniae Glu-tRNA |
| | | Gln Amido-transferase (C subunit) (gatC gene) |
| SEQ ID NO: 11 | CP4 #2 | Polypeptide translation corresponding to SEQ ID |
| | | NO. 10, homolog to C. pneumoniae Glu-tRNA Gln |
| | | Amido-transferase (C subunit) (gatC gene) |
| SEQ ID NO: 12 | CP4 #2 | (full length) homolog to C. pneumoniae Glu-tRNA |
| | | Gln Amido-transferase (C subunit) (gatC gene) |
| SEQ ID NO: 13 | CP4 #2 | Polypeptide translation corresponding to SEQ ID |
| | | NO. 12, homolog to C. pneumoniae Glu-tRNA Gln |
| | | Amido-transferase (C subunit) (gatC gene) |
| SEQ ID NO: 14 | CP4 #3 | (fragment) homolog to C. pneumoniae Glu-tRNA |
| | | Gln Amido-transferase (A subunit) (gatA gene) |
| SEQ ID NO: 15 | CP4 #3 | Polypeptide translation corresponding to SEQ ID |
| | | NO. 14, homolog to C. pneumoniae Glu-tRNA Gln |
| | | Amido-transferase (A subunit) (gatA gene) |
| SEQ ID NO: 16 | CP4 #3 | (full length) homolog to C. pneumoniae Glu-tRNA |
| | | Gln Amido-transferase (A subunit) (gatA gene) |
| SEQ ID NO: 17 | CP4 #3 | Polypeptide translation corresponding to SEQ ID |
| | | NO. 16, homolog to C. pneumoniae Glu-tRNA Gln |
| | | Amido-transferase (A subunit) (gatA gene) |
| SEQ ID NO: 18 | CP4 #3 | (full length) homolog to C. pneumoniae Glu-tRNA |
| | | Gln Amido-transferase (B subunit) (gatB gene) |
| SEQ ID NO: 19 | CP4 #3 | Polypeptide translation corresponding to SEQ ID |
| | | NO. 18, homolog to C. pneumoniae Glu-Trna Gln |
| | | Amido-transferase (B subunit) (gatB gene) |
| SEQ ID NO: 20 | CP4 #4 | (fragment) C. psittaci 90 kDa outer membrane |
| | | protein (OMP90A gene) (Previously sequenced by |
| | | Longbottom, et al) |
| SEQ ID NO: 21 | CP4 #4 | Polypeptide translation corresponding to SEQ ID |
| | | NO. 20, C. psittaci 90 kDa outer membrane protein |
| | | (OMP90A gene) |
| SEQ ID NO: 22 | CP4 #4 | (full length) C. psittaci 90 kDa outer membrane |
| | | protein (OMP90A gene) (Previously sequenced by |
| | | Longbottom, et al) |
| SEQ ID NO: 23 | CP4 #4 | Polypeptide translation corresponding to SEQ ID |
| | | NO. 22, C. psittaci 90 kDa outer membrane protein |
| | | (OMP90A gene) |
| SEQ ID NO: 24 | CP4 #5 | (fragment) homolog to C. pneumoniae |
| | | transglycolase/transpeptidase (pbp3 gene) |
| SEQ ID NO: 25 | CP4 #5 | Polypeptide translation corresponding to SEQ ID |
| | | NO. 24, homolog to C. pneumoniae |
| | | transglycolase/transpeptidase (pbp3 gene) |
| SEQ ID NO: 26 | CP4 #5 | (full length) homolog to C. pneumoniae |
| | | transglycolase/transpeptidase (pbp3 gene) |
| SEQ ID NO: 27 | CP4 #5 | Polypeptide translation corresponding to SEQ ID |
| | | NO. 26, homolog to C. pneumoniae |
| | | transglycolase/transpeptidase (pbp3 gene) |
| SEQ ID NO: 28 | CP4 #6 | (fragment) homolog to C. pneumoniae Protein |
| | | Translocase (secA2 gene) |
| SEQ ID NO: 29 | CP4 #6 | Polypeptide translation corresponding to SEQ ID |
| | | NO. 28, homolog to C. pneumoniae Protein |
| | | Translocase (secA2 gene) |
| SEQ ID NO: 30 | CP4 #13 | (fragment) homolog to C. pneumoniae Protein |
| | | Translocase (secA2 gene) |
| SEQ ID NO: 31 | CP4 #13 | Polypeptide translation corresponding to SEQ ID |
| | | NO. 30, homolog to C. pneumoniae Protein |
| | | Translocase (secA2 gene) |
| SEQ ID NO: 32 | CP4 #6 & 13 | (full length) homolog to C. pneumoniae Protein |
| | | Translocase (secA2 gene) |
| SEQ ID NO: 33 | CP4 #6 & 13 | Polypeptide translation corresponding to SEQ ID |
| | | NO. 32, homolog to C. pneumoniae Protein |
| | | Translocase (secA2 gene) |
| SEQ ID NO: 34 | CP4 #7 | (fragment) homolog to C. pneumoniae Outer |
| | | Membrane Lipoprotein (Cpn 0278) |
| SEQ ID NO: 35 | CP4 #7 | Polypeptide translation corresponding to SEQ ID |
| | | NO. 34, homolog to C. pneumoniae Outer |
| | | Membrane Lipoprotein (Cpn 0278 gene) |
| SEQ ID NO: 36 | CP4 #7 | (full length) homolog to C. pneumoniae Outer |
| | | Membrane Lipoprotein (Cpn 0278) |
| SEQ ID NO: 37 | CP4 #7 | Polypeptide translation corresponding to SEQ ID |
| | | NO. 36, homolog to C. pneumoniae Outer |
| | | Membrane Lipoprotein (Cpn 0278 gene) |
| SEQ ID NO: 38 | CP4 #8 | (fragment) homolog to C. pneumoniae |
| | | Oligopeptidase (pepF gene) |
| SEQ ID NO: 39 | CP4 #8 | Polypeptide translation corresponding to SEQ ID |
| | | NO. 38, homolog to C. pneumoniae Oligopeptidase |
| | | (pepF gene) |
| SEQ ID NO: 40 | CP4 #8 | (fragment) homolog to C. pneumoniae |
| | | Oligopeptidase (pepF gene) |
| SEQ ID NO: 41 | CP4 #8 | Polypeptide translation corresponding to SEQ ID |
| | | NO. 40, homolog to C. pneumoniae Oligopeptidase |
| | | (pepF gene) |
| SEQ ID NO: 42 | CP4 #9 | (fragment) homolog to C. pneumoniae gene of |
| | | unknown function, co-translationaly coupled to |
| | | Yop N Flagellar-Type ATPase (Cpn 0708 gene) |
| SEQ ID NO: 43 | CP4 #9 | Polypeptide translation corresponding to SEQ ID |
| | | NO. 42, homolog to C. pneumoniae gene of |
| | | unknown function, co-translationally coupled to |
| | | Yop N Flagellar-Type ATPase (Cpn 0708 gene) |
| SEQ ID NO: 44 | CP4 #9 | (full length) homolog to C. pneumoniae gene of |
| | | unknown function, co-translationally coupled to |
| | | Yop N Flagellar-Type ATPase (Cpn 0708 gene) |
| SEQ ID NO: 45 | CP4 #9 | Polypeptide translation corresponding to SEQ ID |
| | | NO. 44, homolog to C. pneumoniae gene of |
| | | unknown function, co-translationally coupled to |
| | | Yop N Flagellar-Type ATPase (Cpn 0708 gene) |
| SEQ ID NO: 46 | CP4 #9 | (full length) homolog to C. pneumoniae Yop N |
| | | Flagellar-Type ATPase (yscN gene) |
| SEQ ID NO: 47 | CP4 #9 | Polypeptide translation corresponding to SEQ ID |
| | | NO. 46, homolog to C. pneumoniae Yop N |
| | | Flagellar-Type ATPase (yscN gene) |
| SEQ ID NO: 48 | CP4 #10 | (fragment) homolog to C. pneumoniae outer |
| | | membrane protein G (pmp 2 gene) (Nucleotides 1-423 |
| | | were previously sequenced by Longbottom et |
| | | al.) |
| SEQ ID NO: 49 | CP4 #10 | Polypeptide translation corresponding to SEQ ID |
| | | NO. 48, homolog to C. pneumoniae outer |
| | | membrane protein G (pmp 2 gene) |
| SEQ ID NO: 50 | CP4 #11 | (fragment) homolog to C. pneumoniae outer |
| | | membrane protein G (pmp 2 gene) (Nucleotides 1-301 |
| | | were previously sequenced by Longbottom et |
| | | al.) |
| SEQ ID NO: 51 | CP4 #11 | Polypeptide translation corresponding to SEQ ID |
| | | NO. 50, homolog to C. pneumoniae outer |
| | | membrane protein G (pmp 2 gene) |
| SEQ ID NO: 52 | CP4 #10 & | (full length) homolog to C. pneumoniae outer |
| | 11 | membrane protein G (pmp 2 gene). This gene |
| | | immediately follows the OMP90A gene on C. |
| | | psittaci, and nucleotides 1-502 were published by |
| | | Longbottom et al., although they did not report this |
| | | as a gene. |
| SEQ ID NO: 53 | CP4 #10 & | Polypeptide translation corresponding to SEQ ID |
| | 11 | NO. 52, homolog to C. pneumoniae outer |
| | | membrane protein G (pmp 2 gene). |
| SEQ ID NO: 54 | CP4 #12 | (fragment) C. psittaci 98 kDa outer membrane |
| | | protein (POMP gene) (Previously sequenced by |
| | | Longbottom, et al) |
| SEQ ID NO: 55 | CP4 #12 | Polypeptide translation corresponding to SEQ ID |
| | | NO. 54, C. psittaci 98 kDa outer membrane protein |
| | | (POMP gene) |
| SEQ ID NO: 56 | CP4 #12 | (full length) C. psittaci 98 kDa outer membrane |
| | | protein (POMP gene) (Previously sequenced by |
| | | Longbottom et al.) |
| SEQ ID NO: 57 | CP4 #12 | Polypeptide translation corresponding to SEQ ID |
| | | NO. 56, C. psittaci 98 kDa outer membrane protein |
| | | (POMP gene) |
| SEQ ID NO: 58 | CP4 #14 | (fragment) homolog to C. pneumoniae Succinate |
| | | Dehydrogenase (sdhC) |
| SEQ ID NO: 59 | CP4 #14 | Polypeptide translation corresponding to SEQ ID |
| | | NO. 58, homolog to C. pneumoniae Succinate |
| | | Dehydrogenase (sdhC gene) |
| SEQ ID NO: 60 | CP4 #14 | (full length) homolog to C. pneumoniae Succinate |
| | | Dehydrogenase (sdhC) |
| SEQ ID NO: 61 | CP4 #14 | Polypeptide translation corresponding to SEQ ID |
| | | NO. 60, homolog to C. pneumoniae Succinate |
| | | Dehydrogenase (sdhC gene) |

Of the 14 single genes identified in this study, ten are internal fragments and three contain the C-terminus of the protein. Of the five most protective clones (CP4 #1-5), one was from a putative outer membrane protein (CP4 #4) and one was from a cell surface protein (CP4 #5). The other three were from cytosolic proteins, with CP4 #2 and CP4 #3 deriving independently from genes encoding a particular amidotransferase complex.

Four of the 14 clones have sequence similarity to a class of proteins known as putative outer membrane proteins (POMPs) in Chlamydia psittaci and Chlamydia pneumoniae (CP4 #4, CP4 #10, CP4 #11 and CP4 #12). Many of the “putative” outer membrane proteins are known to be localized to the outer membrane and to be highly immunogenic (Longbottom et al., 1996; Tan et al., 1990). The clone designated CP4 #4 is an in-frame fragment of POMP90A (Longbottom et al., 1998) and CP4 #12 is an in-frame fragment of a 98 kDa POMP which has been completely sequenced (Accession U72499). The clones CP4 #10 and CP4 #11 immediately follow CP4 #4 in the genome and have sequence similarity to POMPs in C. psittaci, C. trachomatis and C. pneumoniae. As stated earlier, the clone CP4 #10 overlaps the CP4 #11 clone. Of these clones only CP4 #4 confers significant protection in isolation so clearly the criteria of being an outer membrane protein is not sufficient to predict a protective vaccine.

### Example 7

### Mixing Experiment

The two dimensional approach used to find protective gene fragments assumes that the protection is due to a single highly protective gene within a pool. To verify that such genes would be found, 25 ng (i.e. 1/2000) of either CP4 #4 or CP4 #11 was added to a pool that scored negative (pool 6 round 1). As depicted in FIG. 7, spiking with either clone converted the negative library to a positive. Of note is that CP4 #11 did not confer protection when tested individually, however, it does protect in combination.

The fact that a CP4 #4 positive library confers protection validates the sensitivity of the system. The fact that a CP4 #11 positive library protects implies that CP4 #11 con be a useful component of a vaccine, but that it may depend upon having other antigens present. A likely explaination is that CP4 #11 is a good vaccine antigen, but requires immunological help.

### Example 8

### Vaccination in Cattle

An important question is whether the genes identified in this manner in a mouse model are clinically relevant. Of course, this concern is not peculiar to genetic vaccines or ELI, but any system that uses models to identify vaccine candidates. In this case the clinically relevant situation is protection of cattle. In a preliminary experiment, the inventors evaluated the pool of 14 individual clones in the original host in a fertility challenge model. All fourteen clones were used as the individual test data on each clone in mice was not available by the time it was necessary to initiate the cow trial. TABLE 3.

TABLE 3
C. psittaci Vaccine in Cows

| | Percent | | Not |
|---|---|---|---|
| | Pregnant | Pregnant | Pregnant |
| Not Challenged | 75 | 3 | 1 |
| Challenged, | 0 | 0 | 4 |
| Not Vaccinated | | | |
| EB Vaccine | 25 | 1 | 3 |
| Genetic | 33 | 2 | 4 |
| Vaccine | | | |
| (14 gene pool) | | | |

C. psittaci is normally introduced by the fecal-oral and respiratory routes in cattle, and disseminates to other tissues including reproductive organs. C. psittaci infection of the uterine mucosa reduces fertility, the basis of the economic interest in a C. psittaci vaccine. Four groups of heifers were used. One group was the naive unchallenged control, another the naive, challenged control, a third received the same pool of fourteen gene fragments that were tested in mice, and the fourth group was vaccinated with an experimental, inactivated vaccine of elementary bodies (EB) and also challenged. This EB vaccine had shown great promise in field trials but is too expensive to produce. After a prime and one boost, the heifers were estrus synchronized by prostaglandin injection, were in heat 2-3 days later, and were artificially inseminated, simultaneously receiving an intracervical chlamydial challenge of 3×10⁷ inclusion forming units. The heifers were palpated for pregnancy at six weeks after insemination. This challenge was very high in order to maximize the difference between positive and negative control animals. This was necessary because only a small number of cows could be justified for this high-risk experiment.

Although the animal numbers are small, the results are quite encouraging. As is seen in Table 1, three out of four animals became pregnant in the positive control (non-challenged) group, 0/4 in the negative control (non-vaccinated, challenged) group, 2/6 in the genetic immunization group, and 1/4 in the elementary body vaccine group. The genetic vaccine of the pooled genes performed at least as well as the EB vaccine. Also relative to the inventor's interest in therapeutic vaccines, these cows were not sterile with respect to C. psittaci at the time of the prime inoculation. The vaccination was in the face of previous exposure and low level C. psittaci infection, as determined by the high titers of preinoculation antichlamydial antibodies, and occasional positivity of Chlamydia omp1 PCRs from vaginal scrapings.

The next phase in developing a cow vaccine will be to experimentally verify the effectiveness of particular groups of the protective genes and then convert the codon usage of the C. psittaci genes to that of a mammal. This should increase the expression of the antigen in cows and increase the effectiveness of the vaccine. The inventors will test different combinations of those genes which have been found to be individually protective, as well as combinations with CP4 #11. Both the original fragments and their full-length versions can be tested, both as nucleic acid segments and proteins. Once the combinations have been verified in mice or other small mammals, those combinations showing the most promise will be tested in cows. After immunization, the cows will be challenged with C. psittaci, either by direct challenge at insemination or infection by herd-mates. Direct challenge at insemination is a very severe and unnatural form of challenge. Therefore, even if protection is not demonstrated in the wake of such challenge, this does not necessarily mean that no protection has been conferred upon the cows.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

U.S. Pat. No. 5,670,488

U.S. Pat. No. 5,770,414

U.S. Pat. No. 5,824,544

U.S. Pat. No. 5,830,725

U.S. Pat. No. 5,851,826

U.S. Pat. No. 5,858,744

U.S. Pat. No. 5,879,934

U.S. Pat. No. 5,888,502

U.S. Pat. No. 5,932,210

U.S. Pat. No. 5,955,331

Amado and Chen, “Lentiviral vectors-the promise of gene therapy within reach?” Science, 285(5428):674-676, 1999.

Amado, Mitsuyasu, Zack, “Gene therapy for the treatment of AIDS: animal models and human clinical experience,” Front Biosci., 4:D468-475, 1999.

Babiuk, L. A. “Broadening the approaches to developing more effective vaccines,” Vaccine 17, 1587-95 (1999).

Banghametal., J. Mol. Biol., 13:238, 1965.

Barry, M. A., Lai, W. C., and Johnston, S. A. Protection against mycoplasma infection using expression-library immunization. Nature 377, 632-5 (1995).

Batra, Guttridge, Brenner, Dubinett, Baldwin, Boucher, “IkappaBalpha gene transfer is cytotoxic to squamous-cell lung cancer cells and sensitizes them to tumor necrosis factor-alpha-mediated cell death,” Am. J. Respir. Cell Mol. Biol., 21(2):238-245, 1999.

Bett, Prevec, Graham, “Packaging capacity and stability of human adenovirus type 5 vectors,” J. Virol., 67(10):5911-5921, 1993.

Blackwell, Miller, Douglas, Li, Peters, Carroll, Peters, Strong, Curiel, “Retargeting to EGFR enhances adenovirus infection efficiency of squamous cell carcinoma,” Arch. Otolaryngol Head Neck Surg., 125(8):856-863, 1999.

Brayton, K. A., Vogel, S. W., and Allsopp, B. A. (1998). Expression library immunization to identify protective antigens from Cowdria ruminantium. Ann N Y Acad Sci 849, 369-71.

Campbell, L. A., C₁-C., K., and Grayston, J. T. (1998). Chlamydia pneumoniae and cardiovascular disease. Emerging Infectious Disease 4, 571-579.

Carrio, Romagosa, Mercade, Mazo, Nadal, Gomez-Foix, Fillat, “Enhanced pancreatic tumor regression by a combination of adenovirus and retrovirus-mediated delivery of the herpes simplex virus thymidine kinase gene,” Gene Ther., 6(4):547-553, 1999.

Case, Price, Jordan, Yu, Wang, Bauer, Haas, Xu, Stripecke, Naldini, Kohn, Crooks, “Stable transduction of quiescent CD34(+)CD38(−) human hematopoietic cells by HIV-1-based lentiviral vectors,” Proc. Natl. Acad. Sci. USA, 96(6):2988-2993, 1999.

Chillon, Bosch, Zabner, Law, Armentano, Welsh, Davidson, “Group D adenoviruses infect primary central nervous system cells more efficiently than those from group C,” J. Virol., 73(3):2537-2540, 1999.

Clay, Custer, Spiess, Nishimura, “Potential use of T cell receptor genes to modify hematopoietic stem cells for the gene therapy fo cancer,” Pathol. Oncol. Res., 5(1):3-15, 1999.

Danesh, J., Collins, R., and Peto, R. (1997). Chronic infections and coronary heart disease: is there a link? [see comments]. Lancet 350, 430-6.

Deamer and Uster, “Liposome Preparation: Methods and Mechanisms,” LIPOSOMES, M. Ostro ed. (1983).

Derby, Sena-esteves, Breakefield, Corey, “Gene transfer into the mammalian inner ear using HSV-1 and vaccinia virus vectors,” Hear Res., 134(1-2):1-8, 1999.

Dongji, Z., Xi, Y., Caixia, S., and Brunham, R. C. (1998). The immune responses following intramuscular DNA immunization with the MOMP gene of Chlamydia trachomatis. In Chlamydial Infections: Proceedings of the 9th International Symposium on Human Chlamydial Infection., e. a. R. Stephens, ed. (Napa, California: University of California Press, Berkeley, USA.), pp. 442-445.

Dorai, Perlman, Walsh, Shabsigh, Goluboff, Olsson, Buttyan, “A recombinant defective adenoviral agent expressing anti-bcl-2 ribozyme promotes apoptosis of bcl-2-expressing human prostate cancer cells,” Int. J. Cancer, 82(6):846-852, 1999.

Ellis, R. W. (1999). New technologies for making vaccines. Vaccine 17, 1596-604.

Engel and Kohn, “Stem cell directed gene therapy,” Front Biosci., 4:e26-33, 1999.

Feldman, Tahlil, Steg, “Adenovirus-mediated arterial gene therapy for restenosis: problems and perspectives,” Semin Interv. Cardiol., 1(3):203-208, 1996.

Feltquate, D. M., Heaney, S., Webster, R. G., and Robinson, H. L. (1997). Different T helper cell types and antibody isotypes generated by saline and gene gun DNA immunization. J Immunol 158, 2278-84.

Fujiwara and Tanaka, “Molecular surgery for human colorectal cancer with tumor suppressor p53 gene transfer,” Nippon Geka gakkai Zasshi, 99(7):463-468, 1998.

Garrido, Carnicero, Lim, Schimmang, “Differential effects on the survival of neuronal and non-neuronal cells after infection by herpes simplex virus type 1 mutants,” J. Neurovirol., 5(30)280-288, 1999.

Ghosh and Bachhawat, “Targeting of liposomes to hepatocytes,” In: Wu G. Wu C ed., Liver diseases, targeted diagnosis and therapy using specific receptors and ligands, New York: Marel Dekker, pp. 87-104, 1991.

Graham and Prevec, “Methods for construction of adenovirus vectors,” Mol. Biotechnol., 3(3):207-220, 1995.

Grayston, J. T., Woolridge, R. L., and Wang, S.-P. (1962). Trachoma vaccine studies on Taiwan. Ann. NY Acad. Sci. 98, 352-362.

Gregoriadis, DRUG CARRIERS IN BIOLOGY AND MEDICINE, G. Gregoriadis (ed.), 1979, pp. 287-341.

Haecker, Stedman, Balice-Gordon, Smith, Greelish, Mitchell, Wells, Sweeney, Wilson, “In vivo expression of full-length human dystrophin from adenoviral vectors deleted of all viral genes,””Hum. Gene Ther., 7(15):1907-1914, 1996.

Hermens and Vergaagen, “Viral vectors, tools for gene transfer in the nervous system,” Prog. Neurobiol., 55(4):399-432, 1998.

Howard, Kalthoff, Fong, “Ablation of tumor cells in vivo by direct injection of HSV-thymidine kinase retroviral vector and ganciclovir therapy,” Ann. N.Y. Acad. Sci., 880-352-365, 1999.

Huang, J., Wang, M.-D., Lenz, S. D., Gao, D., and Kaltenboeck, B. (1999). Interleukin-12 administered during Chlamydia psittaci lung infection in mice confers immediate and long-term protection and reduces MIP-2 level and neutrophil infiltration in lung tissue. J. Immunol. 162, 2217-2226.

Ilan, Saito, Thummala, Chowdhury, “Adenovirus-mediated gene therapy of liver diseases,” Semin Liver Dis., 19(1):49-59, 1999.

Irie, Anderegg, Kashani-Sabet, Ohkawa, Suzuki, Halks-Miller, Curiel, Scanlon, “Therapeutic efficacy of an adenovirus-mediated anti-H-ras ribozyme in experimental bladder cancer,” Antisense Nucleic Acid Drug Dev., 9(4):341-349, 1999.

Johnston, Gasmi, Lim, Elder, Yee, Jolly, Campbell, Davidson, Sauter, “Minimum requirements for efficient trandsduction of dividing and nondividing cells by feline immunodeficiency virus vectors,” J. Virol., 73(6):4991-5000, 1999.

Kalman, S., Mitchell, W., Marathe, R., Lammel, C., Fan, J., Hyman, R. W., Olinger, L., Grimwood, J., Davis, R. W., and Stephens, R. S. (1999). Comparative genomes of Chlamydia pneumoniae and C. trachomatis. Nature Genetics 21, 385-389.

Kaltenbock, B., Schmeer, N., and Schneider, R. (1997). Evidence for numerous omp1 alleles of porcine Chlamydia trachomatis and novel chlamydial species obtained by PCR. J Clin Microbiol 35, 1835-41.

Kaltenboeck, B., Huang, J., Wang, M.-D., Lenz, S. D., and Gao, D. (1998). Genetically determined vigorous innate immunity is associated with protection against primary chlamydial lung infection in mice, but with profound disease exacerbation in reinfection. In Chlamydial Infections: Proceedings of the 9th International Symposium on Human Chlamydial Infection, e. a. R. Stephens, ed. (Napa, California: University of California Press).

Kaltenboeck, B., Kousoulas, K. G., and Storz, J. (1993). Structures of and allelic diversity and relationships among the major outer membrane protein (ompA) genes of the four chlamydial species. J Bacteriol 175, 487-502.

Kaneda et al., “Increased expression of DNA cointroduced with nuclear protein in adult rat liver,” Science, 243:375-378, 1989.

Kato et al., “Expression of hepatitis B virus surface antigen in adult rat liver,” J. Biol. Chem., 266:3361-3364, 1991.

Kaufman, Jia, Tan, Chen, Gabelt, Booth, Tufaro, Cynader, “A perspective of gene therapy in the glaucomas,” Surv. Ophthalmol., 43(1S):91-97, 1999.

Kay, “Hepatic gene therapy for haemophilia B.,” Haemophilia, 4(4):389-392, 1998.

Klimatcheva, Rosenblatt, Planelles, “Lentiviral vectors and gene therapy,” Front Biosci., 4:D481-496, 1999.

Lachmann and Efstathiou, “Use of herpes simplex virus type 1 for transfene expression within the nervous system,” Clin. Sci., 96(6):533-541, 1999.

Leibowitz, Beattie, Kafri, Cirulli, Lopez, Hayek, Levine, “Gene transfer to human pancreatic endocrine cells using viral vectors,” 48(4):745-753, 1999.

Lesch, “Gene transfer to the brain: emerging therapeutic strategy in psychiatry,” Biol. Psychiatry, 45(3):247-253, 1999.

Longbottom, D., Russell, M., Dunbar, S. M., Jones, G. E., and Herring, A. J. (1998).

Molecular cloning and characterization of the genes coding for the highly immunogenic cluster of 90-kilodalton envelope proteins from the Chlamydia psittaci subtype that causes abortion in sheep. Infect Immun 66, 1317-24.

Longbottom, D., Russell, M., Jones, G. E., Lainson, A., and Herring, A. J. (1996). Identification of a multigene familiy coding for the 90 kDa proteins of the ovine abortion subtype of Chlamydia psittaci. FEMS Microbiology Letters 142, 277-281.

Marienfeld, Haack, Thalheimer, Schneider-Rasp, Brackmann, Poller, “Autoreplication of the vector genome in recombinant adenorviral vectors with different El region deletions and transgenes, Gene Ther., 6(6):1101-1113, 1999.

Miller, Miller, Garcia, Lynch, “Use of retroviral vectors for gene transfer and expression,” Methods Enzymol., 217:581-599, 1993.

Miyake, Suzuki, Matsuoka, Tohyama, Shimada, “Stable integration of human immunodeficiency virus-based retroviral vectors into the chromosomes of nondividing cells,” Hum. Gene Ther., 9(4):467-475, 1998.

Miyatake, Tani, Feigenbaum, Sundaresan, Toda, Narumi, Kikuchi, Hashimoto, Hangai, Martuza, Rabkin, “Hepatoma-specific antitumor activity of an albumin enhancer/promoter regulated herpes simplex virus in vivo, Gene Ther., 6(4):564,572, 1999.

Moldawer, Edwards, Josephs, Minter, Copeland, MacKay, “Application of gene therapy to acute inflammatory diseases,” Shock, 12(2):83-101, 1999.

Morrison, Onions, Nicolson, “Complete DNA sequence of canine adenovirus type 1,” J. Gen. Virol.,78(Pt4):873-878, 1997.

Morrison, R. P., Feilzer, K., and Tumas, D. B. (1995). Gene knockout mice establish a primary protective role for major histocmpatibility complex class II-restricted responses in Chlamydia trachomatis genital tract infection. Infect. Immun. 63, 4661-4668.

Morrison, R. P., J., B. R., Lyng, K., and Caldwell, H. D. (1989). Chlamydia disease pathogenesis. The 57-kDa chlamydial hypersensitivity antigen is a stress response protein. J Exp Med 170, 1271.

Morrison, R. P., Lyng, K., and Caldwell, H. (1989). Chlamydial disease pathogenesis: ocular hypersensitivity elicited by a genus-specific 57-kD protein. J. Exp. Med. 169, 663-675.

Muhlestein, J. B. (1998). Bacterial infections and atherosclerosis. J. Invest. Med. 46, 396-402.

Nicolau et al., “Liposomes as carriers for in vivo gene transfer and expression,” Methods Enzymol., 149:157-176, 1987.

Pal, S., Barnhart, K. M., Wei, Q., Abai, A. M., Peterson, E. M., and de la Maza, L. M. (1999). Vaccination of mice with DNA plasmids coding for the Chlamydia trachomatis major outer membrane protein elicits an immune response but fails to protect against a genital challenge. Vaccine 17, 459-65.

Petrof, “Respiratory muscles as a target for adenovirus-mediated gene therapy,” Eur. Respir. J., 11(2):492-497, 1998.

Piedrafita, D., Xu, D., Hunter, D., Harrison, R. A., and Liew, F. Y. (1999). Protective immune response induced by vaccination with an expression genomic library of Leishmania major. Journal of Immunology 163, 1467-1472.

Quinn (1998). Does Chlamydia pneumoniae cause coronary heart disease? Current Opinions in Infectious Diseases 11, 301-307.

Rabinovitch, Suarez-Pinzon, Strynadka, Ju, Edelstein, Brownlee, Korbutt, Rajotte, “Transfection of human pancreatic islets with an anti-apoptotic gene (bcl-2) protects beta-cells from cytokine-induced destruction,” Diabetes, 48(6):1223-1229, 1999.

Reddy, Idamakanti, Zakhartchouk, Baxi, Lee, Pyne, Babiuk, Tikoo, “Nucleotide sequence, genome organization, and transcription map of bovine adenovirus type 3,” J. Virol., 72(2):1394-1402, 1998.

Robbins, Tahara, Ghivizzani, “Viral vectors for gene therapy,” Trends Biotech., 16(1):35-40, 1998

Rottenberg, M. E., Gigliotti Rothfuchs, A. C., Gigliotti, D., Svanholm, C., Bandtholtz, L., and Wigzell, H. (1999). Role of innate and adaptive immunity in the outcome of primary infection with Chlamydia pneumoniae, as analyzed in genetically modified mice. J. Immunol., 2829-2836.

Sanford, J. C., Devit, M. J., Russel, J. A., Smith, F. D., Harpending, P. R., Roy, M. K., and Johnston, S. A. (1991). An improved, helium-driven biolistic device. Technique 3, 3-16.

Schachter, J. (1988). Overview of human diseases. In Microbiology of Chlamydia, A. L. Barron, ed. (Boca Raton, Fla.: CRC Press, Inc.), pp. 153-165.

Smith, “Adenovirus-mediated gene transfer to treat neurologic disease,” Arch. Neurol., 55(8):1061-1064, 1998.

Stamm, W. E. (1999). Chlamydia trachomatis infections: progress and problems. J. Inf. Dis. 179 Suppl 2, S380-383.

Stewart, Lassam, Quirt, Bailey, Rotstein, Krajden, Dessureault, Gallinger, Cappe, Wan, Addison, Moen, Gauldie, Graham, “Adenovector-mediated gene delivery of interleukin-2 in metastatic breast cancer and melanoma: results of a phase 1 clinical trial,” Gene Ther., 6(3):350-363, 1999.

Storz, J. (1966). Antigenic structures and interrelations of PL agents associated with polyarthritis, enzootic abortion, intrauterine and latent intestinal infections. J. Comp. Pathol. 76, 351.

Storz, J., and Kaltenboeck, B. (1993). Disease diversity of chlamydial infections. In Rickettsial and chlamydial diseases of domestic animals, Z. Woldehiwet and M. Ristic, eds. (Oxford, UK: Pergamon Press), pp. 363-392.

Sykes, K. F., and Johnston, S. A. (1999). Genetically-live vaccines mimic the antigenicity but not pathogenicity of live viruses. DNA Cell. Biol. 18, 521-531.

Szoka and Papahadjopoulos, Proc. Nat'l Acad. Sci. U.S.A. 75:4194-98 (1978).

Takahashi, Miyoshi, Verma, Gage, “Rescue from photoreceptor degeneration in the rd mouse by human immunodeficiency virus vector-mediated gene transfer,” J. Virol., 73(9):7812-7816, 1999.

Tan, T. W., Herring, A. J., Anderson, I. E., and Jones, G. E. (1990). Protection of sheep against Chlamydia psittaci infection with a subcellular vaccine containing the major outer membrane protein. Infect Immun 58, 3101-8.

Tang, D. C., DeVit, M., and Johnston, S. A. (1992). Genetic immunization is a simple method for eliciting an immune response. Nature 356, 152-4.

Vanderkwaak, Wang, Gomez-Navarro, Rancourt, Dmitriev, Krasnykh, Barnes, Siegal, Alvarez, Curiel, “An advanced generation of adenoviral vectors selectively enhances gene transfer for ovarian cancer gene therapy approaches,” Gynecol. Oncol., 74(2):227-234, 1999.

Vanrompay, D., Cox, E., Vandenbussche, F., Volckaert, G., and Goddeeris, B. (1999). Protection against Chlamydia psittaci challenge by gene gun-based DNA immunizations. Vaccine 17, 2628-2635.

Wang, R., Doolan, D. L., Charoenvit, Y., Hedstrom., R. C., Gardner, M. J., Hobart, P., Tine, J., Sedegah, M., Fallarme, V., Sacci jr., J. B., Kaur, M., Klinman, D. M., Hoffman, S. L., and Weiss, W. R. (1998). Simultaneous induction of multiple antigen-specific cytoxic T lymphocytes in nonhuman primates by immunization with a mixture of four Plasmodium falciparum DNA plasmids. Infec. Immun. 66, 4193-4202.

Weihl, Macdonald, Stoodley, Luders, Lin, “Gene therapy for cerebrovascular disease,” Neurosurgery, 44(2):239-252, 1999.

White, Renda, Nam, Klimatcheva, Zhu, Fisk, Halterman, Rimel, Federoff, Pandya, Rosenblatt, Planelles, “Lentivirus vectors using human and simian immunodeficiency virus elements,” J. Virol., 73(4):2832-2840, 1999.

Wilson, “Adenoviruses as gene-delivery vehicles,” N. Engl. J. Med., 334(18):1185-1187, 1996.

Wong et al., “Appearance of β-lactamase activity in animal cells upon liposome mediated gene transfer,” Gene, 10:87-94, 1980.

Wu, “Recent advances in gene therapy of GI and liver diseases,” 39(5):297-300, 1998.

Yeung, Bockhold, Tufaro, “Efficient infection of mature skeletal muscle with herpes simplex virus vectors by using dextran sulfate as a co-receptor,” Gene Ther., 6(9):1536-1544, 1999.

Yoon, Carroll, Chiocca, Tanabe, “Influence of p53 on herpes simplex virus type 1 vectors for cancer gene therapy,” J. Gastrointest. Surg., 3(10):34-48, 1999.

Zheng, Graham, Prevec, “Transcription units of E1a, E1b and pIX regions of bovine adenovirus type 3,” J. Gen. Virol., 80(Pt7):1735-1742, 1999.

What is claimed is:

1. An isolated polynucleotide comprising a region having a sequence of SEQ ID NO:6, SEQ ID NO:8, SEQ ID NO:10, SEQ ID NO:12, SEQ ID NO:14, SEQ ID NO:16, 5 SEQ ID NO:18, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:58, or SEQ ID NO:60, or fragment thereof.

2. An isolated polynucleotide comprising a region having a sequence having at least 17 contiguous nucleotides in common with at least one of SEQ ID NO:6, SEQ ID NO:8, SEQ ID NO:10, SEQ ID NO:12, SEQ ID NO:14, SEQ ID NO:16, SEQ ID NO:18, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID 15 NO:44, SEQ ID NO:46, SEQ ID NO:58, or SEQ ID NO:60, or its complement.

3. The isolated polynucleotide of claim 2, further defined as comprising a sequence having least 50 contiguous nucleotides in common with at least one of SEQ ID NO:6, SEQ ID NO:8, SEQ ID NO:10, SEQ ID NO:12, SEQ ID NO:14, SEQ ID NO:16, SEQ ID NO:18, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:58, or SEQ ID NO:60, or its complement.

4. The isolated polynucleotide of claim 3, further defined as comprising a sequence having all nucleotides in common with at least one of SEQ ID NO: 6, SEQ ID NO:8, SEQ ID NO:10, SEQ ID NO:12, SEQ ID NO:14, SEQ ID NO:16, SEQ ID NO:18, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:58, or SEQ ID NO:60, or its complement.

5. A polypeptide having a sequence of SEQ ID NO:7, SEQ ID NO:9, SEQ ID NO:11, SEQ ID NO:13, SEQ ID NO:15, SEQ ID NO:17, SEQ ID NO:19, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:59, SEQ ID NO:61 or fragment thereof.

6. The polypeptide of claim 5, further defined as a recombinant polypeptide.

7. A method of producing a polypeptide having a sequence of SEQ ID NO:7, SEQ ID NO:9, SEQ ID NO:1, SEQ ID NO:13, SEQ ID NO:15, SEQ ID NO:17, SEQ ID NO:19, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:59, SEQ ID NO:61 or fragment thereof, comprising:
a) obtaining a polynucleotide comprising a region encoding the sequence; and
b) expressing the polynucleotide to obtain the polypeptide.

8. The method of claim 7, wherein the polynucleotide has a region having a sequence of SEQ ID NO:6, SEQ ID NO:8, SEQ ID NO:10, SEQ ID NO:12, SEQ ID NO:14, SEQ ID NO:16, SEQ ID NO:18, SEQ ID NO:24, SEQ ID NO:26, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:58, or SEQ ID NO:60, or fragment thereof

9. An antibody directed against an antigen having sequence of SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, or SEQ ID NO:61, or antigenic fragment thereof.

10. The antibody of claim 9, further defined as a monoclonal antibody.

11. A vaccine for the immunization of a bovine against Chlamydia psittaci comprising:
(a) a pharmaceutically acceptable carrier, and
(b) at least one polynucleotide having a Chlamydia psittaci sequence.

12. The vaccine of claim 1 1, wherein the at least one polynucleotide has a sequence isolated from a Chlamydia psittaci genomic DNA expression library

13. The vaccine of claim 11, wherein the at least one polynucleotide has a sequence of SEQ ID NO:6, SEQ ID NO:8, SEQ ID NO:11, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO52:, SEQ ID NO:54, SEQ ID NO:56, SEQ ID NO: 58, or SEQ ID NO:60, or fragment thereof.

14. The vaccine of claim 13, wherein the at least one polynucleotide has a sequence of SEQ ID NO:6, SEQ ID NO:8, SEQ ID NO:10, SEQ ID NO:12, SEQ ID NO:14, SEQ ID NO:16, SEQ ID NO:20, SEQ ID NO:22, SEQ ID NO:24, or SEQ ID NO:26, or fragment thereof.

15. The vaccine of claim 13, wherein the at least one polynucleotide has a sequence of SEQ ID NO:6, SEQ ID NO:10, SEQ ID NO:14, SEQ ID NO:20, or SEQ ID NO:24.

16. The vaccine of claim 11, wherein the polynucleotide is comprised in a genetic immunization vector.

17. The vaccine of claim 16, wherein the vector comprises a gene encoding a mouse ubiquitin fusion polypeptide.

18. The vaccine of claim 16, wherein the vector comprises a promoter operable in eukaryotic cells.

19. The vaccine of claim 18, wherein the promoter is a CMV promoter.

20. The vaccine of claim 11, wherein the polynucleotide is cloned into a viral expression vector.

21. The vaccine of claim 20, wherein the viral expression vector is selected from the group consisting of adenovirus, adeno-associated virus, retrovirus and herpes-simplex virus.

22. The vaccine of claim 11, comprising a polynucleotide encoding a antigen having a sequence of SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, or SEQ ID NO:61, or antigenic fragment thereof.

23. The vaccine of claim 11, comprising at least a first polynucleotide having a Chlamydia psittaci sequence and a second polynucleotide having a Chlamydia psittaci sequence, wherein the first polynucleotide and the second polynucleotide have different Chlamydia psittaci sequences.

24. The vaccine of claim 23, wherein the first polynucleotide has a sequence of SEQ ID NO:50.

25. A vaccine for the immunization of a bovine against Chlamydia psittaci comprising:
(a) a pharmaceutically acceptable carrier; and
(b) at least one Chlamydia psittaci antigen.

26. The vaccine of claim 25, wherein the at least one Chlamydia psittaci antigen has a sequence of SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO=35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, or SEQ ID NO:61, or antigenic fragment thereof.

27. The vaccine of claim 25, wherein the at least one Chlamydia psittaci antigen has a sequence of SEQ ID NO:7, SEQ ID NO:9, SEQ ID NO:11, SEQ ID NO:13, SEQ ID NO:15, SEQ ID NO:17, SEQ ID NO:21, SEQ ID NO:23, SEQ ID NO:25, or SEQ ID NO:27, or an antigenic fragment thereof.

28. The vaccine of claim 25, wherein the at least one Chlamydia psittaci antigen has a sequence of SEQ ID NO:7, SEQ ID NO:11, SEQ ID NO:15, SEQ ID NO:21, or SEQ ID NO:25.

29. A method of immunizing a bovine comprising providing to the bovine at least one Chlamydia psittaci antigen, or antigenic fragment thereof, in an amount effective to induce an immune response.

30. The method of claim 29, wherein the at least one Chlamydia psittaci antigen has a sequence of SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, or SEQ ID NO:61, or an antigenic fragment thereof.

31. The method of claim 29, wherein the provision of the at least one Chlamydia psittaci antigen comprises:
(a) preparing a cloned expression library from fragmented genomic DNA, cDNA or sequenced genes of Chlamydia psittaci;
(b) administering at least one clone of the library in a pharmaceutically acceptable carrier into the bovine; and
(c) expressing at least one Chlamydia psittaci antigen in the bovine.

32. The method of claim 31, wherein the expression library comprises at least one or more polynucleotide having a sequence of SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO52:, SEQ ID NO:54, SEQ ID NO:56, SEQ ID NO: 58, or SEQ ID NO:60, or fragment thereof.

33. The method of claim 32, wherein the expression library is cloned in a genetic immunization vector of SEQ ID NO: 1.

34. The method of claim 33, wherein the vector comprises a gene encoding a mouse ubiquitin fusion polypeptide designed to link the expression library polynucleotides to the ubiquitin gene.

35. The method of claim 34, wherein the vector comprises a promoter operable in eukaryotic cells.

36. The method of claim 35, wherein the promoter is a CMV promoter.

37. The method of claim 32, wherein the polynucleotide is administered by a intramuscular injection or epidermal injection.

38. The method of claim 37, wherein the polynucleotide is administered by intravenous, subcutaneous, intralesional, intraperitoneal, oral or inhaled routes of administration.

39. The method of claim 38, wherein a second intramuscular injection and epidermal injection are administered at least about three weeks after the first injection.

40. The method of claim 32, wherein the polynucleotide is cloned into a viral expression vector.

41. The method of claim 29, wherein the provision of the Chlamydia psittaci antigen(s) comprises:
(a) preparing a pharmaceutical composition comprising at least one polynucleotide having a sequence of SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO52:, SEQ ID NO:54, SEQ ID NO:56, SEQ ID NO: 58, or SEQ ID NO:60, or fragment thereof;
(b) administering one or more clones of the library in a pharmaceutically acceptable carrier into the bovine; and
(c) expressing one or more Chlamydia psittaci antigens in the bovine.

42. The method of claim 41, wherein the one or more polynucleotides is in one or more expression vectors.

43. The method of claim 42, wherein the one or more polynucleotides are cloned in a genetic immunization vector of SEQ ID NO:1.

44. The method of claim 29, wherein the provision of the Chlamydia antigen(s) comprises:
(a) preparing a pharmaceutical composition of at least one Chlamydia antigen having a sequence of SEQ ID NO:7, SEQ ID NO:9, SEQ ID NO: 11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, or SEQ ID NO:61, or an antigenic fragment thereof; and
(b) administering the at least one antigen or fragment into the bovine.

45. The method of claim 44, wherein the antigen(s) is administered by a first intramuscular injection, intravenous injection, parenteral injection, epidermal injection, inhalation or oral route.

46. The method of claim 45, wherein a second intramuscular injection, intravenous injection, parenteral injection or epidermal injection is administered at least about three weeks after the first injection.

47. A method of assaying for the presence of Chlamydia psittaci infection in a bovine comprising:
(a) obtaining an antibody directed against a Chlamydia psittaci antigen;
(b) obtaining a sample from the bovine;
(c) admixing the antibody with the sample; and
(d) assaying the sample for antigen-antibody binding,
wherein the antigen-antibody binding indicates Chlamydia psittaci infection in the bovine.

48. The method of claim 47, wherein the antibody directed against the antigen is further defined as a monoclonal antibody.

49. The method of claim 47, wherein assaying the sample for antigen-antibody binding is by precipitin reaction, radioimmunoassay, ELISA, Western blot or immunofluorescence.

50. A kit for assaying a Chlamydia psittaci infection comprising, in a suitable container:
(a) a pharmaceutically acceptable carrier; and
(b) an antibody directed against a Chlamydia psittaci antigen.

51. A method of assaying for the presence of a Chlamydia psittaci infection in a bovine comprising:
(a) obtaining an oligonucleotide probe comprising a sequence comprised within one of SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO52, SEQ ID NO:54, SEQ ID NO:56, SEQ ID NO: 58, or SEQ ID NO:60, or a complement thereof, and
(b) employing the probe in a PCR detection protocol.


SEQUENCE LISTING (61 sequences)

SEQ ID NO: 1 - DNA, 127 bp, Artificial Sequence
  Description of Artificial Sequence  Synthetic Primer
ctgcacctggtccttcgcctgagaggtgcagatcttggatcctaagtaagtaagcttgcatgcctgcaggtcgactctaggtgactaatatctagaggatcgatcccgggtggcatccctgtgaccc

SEQ ID NO: 2 - DNA, 15 bp, Artificial Sequence
  Description of Artificial Sequence  Synthetic Primer
gatctggatcccgat

SEQ ID NO: 3 - DNA, 11 bp, Artificial Sequence
  Description of Artificial Sequence  Synthetic Primer
atcgggctcca

SEQ ID NO: 4 - DNA, 19 bp, Artificial Sequence
  Description of Artificial Sequence  Synthetic Primer
ccgcaccctctctgattac

SEQ ID NO: 5 - DNA, 17 bp, Artificial Sequence
  Description of Artificial Sequence  Synthetic Primer
ctggagtggcaacttcc

SEQ ID NO: 6 - DNA, 449 bp, Artificial Sequence
  Description of Artificial Sequence  Synthetic Primer
gaatgtattcgcacgcaaaaatacgctgaagctttgcttcctgtcacgacagcgatcaattctggagtcgcgcctatcaccttcctccatgacctcactgttttttatcgcgatgtactgctaaacaaagatcagggaaattctcctctatcggccatcgccatgcactattccagtgaatgtttattagaaatcattgatttccttggtgaagcggccaaacatctacaacaaactatttttgaaaaaacatttttagaaacagtcatcatccatcttattcggatatgccaacgtccctctttagaaactctgttttctcaactgaaaacatccacgtttgatacagtgagaaacgtaccccagcagcaagaaccctcgaaaccgagtatacaacctgaaaaacactatcaagatcagagtttcttaacttcaccttctcccacgcc

SEQ ID NO: 7 - protein, 149 aa, Chlamydia psittaci
ECIRTQKYAEALLPVTTAINSGVAPITFLHDLTVFYRDVLLNKDQGNSPLSAIAMHYSSECLLEIIDFLGEAAKHLQQTIFEKTFLETVIIHLIRICQRPSLETLFSQLKTSTFDTVRNVPQQQEPSKPSIQPEKHYQDQSFLTSPSPT

SEQ ID NO: 8 - DNA, 1332 bp, Chlamydia psittaci
atgacatcagcaacataccaagtttcttctagaaaataccgccctcaaacatttgccgaaatgctggggcaagatgccgtggtcactgttttaaaaaatgctttgcagtttcaacgtgtcgcgcatgcgtatttattttcagggattcgcggaacaggaaaaacaactttagcaagaatctttgcaaaagccttaaactgtaaagagctgactcctgaacatgaaccatgcaaccagtgttgtgtttgtaaagaaatctcttcaggaacctccttagacgtgatcgaaatcgatggtgcctcgcaccgaggtattgaagatatccgtcaaatcaatgaaaccgtgctctttactcctgccaaatcacaatataaaatctatatcatagatgaagtccatatgctgactaaggaggcgtttaattccttactcaaaactttagaagagcctccgagccatgtaaaattcttcttagcgactacagaaaattataaaatacccagcaccattttaagtcgttgtcaaaaaatgcacctaaagagaattcctgagacaatgattgtagataagctagcatccatatctcaagcaggtgggatagaaacctctcgagaagctcttcttcctattgctagagcagcacagggaagcttacgcgatgctgaatctctttatgattatgtcatagggttattccctacatctttatccccagagttggttgcagacgcattaggtttattatctcaagacaccttagctacattatcagaatgtattcgcacgcaaaaatacgctgaagctttgcttcctgtcacgacagcgatcaattctggagtcgcgcctatcaccttcctccatgacctcactgttttttatcgcgatgtactgctaaacaaagatcagggaaattctcctctatcggccatcgccatgcactattccagtgaatgtttattagaaatcattgatttccttggtgaagcggccaaacatctacaacaaactatttttgaaaaaacatttttagaaacagtcatcatccatcttattcggatatgccaacgtccctctttagaaactctgttttctcaactgaaaacatccacgtttgatacagtgagaaacgtaccccagcagcaagaaccctcgaaaccgagtatacaacctgaaaaacactatcaagatcagagtttcttaacttcaccttctcccacgccaaaagttcagcatcaaaaagaagcttccccttctttagtgggatcagctactatagatacgcttttacaatttgctgttgttgagttttccggaattttaaccaaggagtaa

SEQ ID NO: 9 - protein, 443 aa, Chlamydia psittaci
MTSATYQVSSRKYRPQTFAEMLGQDAVVTVLKNALQFQRVAHAYLFSGIRGTGKTTLARIFAKALNCKELTPEHEPCNQCCVCKEISSGTSLDVIEIDGASHRGIEDIRQINETVLFTPAKSQYKIYIIDEVHMLTKEAFNSLLKTLEEPPSHVKFFLATTENYKIPSTILSRCQKMHLKRIPETMIVDKLASISQAGGIETSREALLPIARAAQGSLRDAESLYDYVIGLFPTSLSPELVADALGLLSQDTLATLSECIRTQKYAEALLPVTTAINSGVAPITFLHDLTVFYRDVLLNKDQGNSPLSAIAMHYSSECLLEIIDFLGEAAKHLQQTIFEKTFLETVIIHLIRICQRPSLETLFSQLKTSTFDTVRNVPQQQEPSKPSIQPEKHYQDQSFLTSPSPTPKVQHQKEASPSLVGSATIDTLLQFAVVEFSGILTKE

SEQ ID NO: 10 - DNA, 123 bp, Chlamydia psittaci
gagtttattcaagagtatgaaagttctttaaatgaagtcattaaaactatggcagcatccatcgctatggatgtaaccgacgtggttattgaggttggtttatcccatgtgatcagtcccgaa

SEQ ID NO: 11 - protein, 41 aa, Chlamydia psittaci
EFIQEYESSLNEVIKTMAASIAMDVTDVVIEVGLSHVISPE

SEQ ID NO: 12 - DNA, 303 bp, Chlamydia psittaci
atgacacaaccctatgtaactagagaagacattatacttctggcgaagagttcagctctggaattaagcgaagagtttattcaagagtatgaaagttctttaaatgaagtcattaaaactatggcagcatccatcgctatggatgtaaccgacgtggttattgaggttggtttatcccatgtgatcagtcccgaagatttacgagaagatatcgttgcctcaagtttctctcgtgaggagtttctaactaatgtccctgaatccttagggggattagtaaaagtacccacagtcattaagtag

SEQ ID NO: 13 - protein, 100 aa, Chlamydia psittaci
MTQPYVTREDIILLAKSSALELSEEFIQEYESSLNEVIKTMAASIAMDVTDVVIEVGLSHVISPEDLREDIVASSFSREEFLTNVPESLGGLVKVPTVIK

SEQ ID NO: 14 - DNA, 514 bp, Chlamydia psittaci
gaaaagtgtgatgtgattgcgatgcctgtatgctcatgcccagcattcgccgatggcgaaatccttgatcctacctctctatatctccaggatatctataccgtggctatgaatttagcctacctcccagctatcgccgttccttcagggttttctcgagaagggctgcctctaggattccaggtgattggacaaaagggtaaagatcaacaggtgtgccaggtaggctatagcttccaagaacattcaggaattaagaatttataccctaaaggatgtaacaaacttgttgatggagaggtgaaataatgagcgacgtttatgctgattgggaatccgtcataggtcttgaagtccacgtagaattaaacacaaaatctaaattgttcagttgtgcacgcaaccgttttggagacgaacctaatacaaacatctctcctgtatgcaccggcatgccggggtcactgccagtactgaataaagaagcagtgagaaaggctgttttatttggttg

SEQ ID NO: 15 - protein, 102 aa, Chlamydia psittaci
EKCDVIAMPVCSCPAFADGEILDPTSLYLQDIYTVAMNLAYLPAIAVPSGFSREGLPLGFQVIGQKGKDQQVCQVGYSFQEHSGIKNLYPKGCNKLVDGEVK

SEQ ID NO: 16 - DNA, 1476 bp, Chlamydia psittaci
atgtatcagaagagtgccttagagttaagaaatgccgtagtgagtggagagtcttcagctacagcaatagcaaagtatttttataatagaataaaaacagaagacaatcagataggagcttttctttctctttgtgaagaaagagcttatgagaaagcagctatcatagatgcgaaagtggcgcgaggagaacctttggggaaactcgcaggtgtccccatcgggataaaagataatattcatattcggggtttgcgcaccacttgtgcttctaaaatgttagaaaattatatagcgccttttgatgctacagtcgtcgaacggatagaagctgaagatggggtcattttaggcaaactcaatatggatgagtttgctatgggatcgacaacgcagtattctgctttccatcctacgaaaaatccttggggtttatcctgtgtgccaggaggatcttcagggggatccgccgccgcagtttctgcaagattttgtcctatagcgttaggttcggataccggtggatctatacgtcagccagcagcattttgtggagttgtggggtttaagccctcctatggagccgtctcccgttacggtttagtcgcttttgggtcttcattagatcagataggccctttaacaacagttgtcgaagatgtcgccttagctatggatgtattcgcaggtaaggatgatagagatgcaacttctcagaagttttttacaggatctttccaagaggccttgtctttagacgttccgagtttgatcggcgtgcctatgggatttttagacggtttacgtgatgatgttaaagagaatttctttgcctctttaagtattttggaacgtcagggtagccgcattgttgaagtggatcttaacatcttagatcacgctgtctctgtttactacattgtcgcttctgcagaagccgcaacaaatcttgcaagatttgatggtattcgttacggctatcgttctccagaagcgcatagtatagaagatatttatacgatctcccgcgtacaaggcttcggtaaggaagtcatgcgtaggattcttttaggtaactatgtgttatccactgagcgccaaaatgtctattataagaaaggctccgcaattcgagcaaaaatcattcaagcttttcaaaaagcttatgaaaagtgtgatgtgattgcgatgcctgtatgctcatgcccagcattcgccgatggcgaaatccttgatcctacctctctatatctccaggatatctataccgtggctatgaatttagcctacctcccagctatcgccgttccttcagggttttctcgagaagggctgcctctaggattccaggtgattggacaaaagggtaaagatcaacaggtgtgccaggtaggctatagcttccaagaacattcaggaattaagaatttataccctaaaggatgtaacaaacttgttgatggagaggtgaaataa

SEQ ID NO: 17 - protein, 491 aa, Chlamydia psittaci
MYQKSALELRNAVVSGESSATAIAKYFYNRIKTEDNQIGAFLSLCEERAYEKAAIIDAKVARGEPLGKLAGVPIGIKDNIHIRGLRTTCASKMLENYIAPFDATVVERIEAEDGVILGKLNMDEFAMGSTTQYSAFHPTKNPWGLSCVPGGSSGGSAAAVSARFCPIALGSDTGGSIRQPAAFCGVVGFKPSYGAVSRYGLVAFGSSLDQIGPLTTVVEDVALAMDVFAGKDDRDATSQKFFTGSFQEALSLDVPSLIGVPMGFLDGLRDDVKENFFASLSILERQGSRIVEVDLNILDHAVSVYYIVASAEAATNLARFDGIRYGYRSPEAHSIEDIYTISRVQGFGKEVMRRILLGNYVLSTERQNVYYKKGSAIRAKIIQAFQKAYEKCDVIAMPVCSCPAFADGEILDPTSLYLQDIYTVAMNLAYLPAIAVPSGFSREGLPLGFQVIGQKGKDQQVCQVGYSFQEHSGIKNLYPKGCNKLVDGEVK

SEQ ID NO: 18 - DNA, 1464 bp, Chlamydia psittaci
atgagcgacgtttatgctgattgggaatccgtcataggtcttgaagtccacgtagaattaaacacaaaatctaaattgttcagttgtgcacgcaaccgttttggagacgaacctaatacaaacatctctcctgtatgcaccggcatgccggggtcactgccagtactgaataaagaagcagtgagaaaggctgttttatttggttgtgctgttgaaggcgaagtagctttgctcagccgttttgatagaaagtcctatttttatcccgatagcccaaggaattttcaaattacccaattcgaacatcctattgtgcgaggaggacatataaaagctatcgttcacggtgaggaacgtcattttgaactggctcaagcgcatatcgaagatgatgccggtatgctaaaacatttcggagaatttgctggagtagattataaccgcgctggtgtacctttaatagagattgtgtctaagccgtgcatgttttgtgctgatgatgctgttgcttatgccacagctttggtatccttattagactacataggcatttctgactgtaatatggaagaaggctcggtacgctttgatgtaaacatatccgtacgtcctaaaggtagcgaagaactacgcaataaagtagaaattaaaaatatgaactcctttgcttttatggcccaagctctagaagccgagcgttgtcgtcagatcgatgcatatttagacaatccaaatgcagaccccaaaactgttattccaggagcgacataccgttgggatcctgaaaagaaaaaaacagtgttgatgcgtcttaaggaacgagctgaagattacaagtatttcatagagcctgatctcccagtattgcaattaacagaagcatatattgatgaaattcgtcatacgcttcccgagctccctttcaacaaataccaaaggtatttgcacgaatatgctcttgccgaagacatcgctgccattttaattagcgataagcatagtgcgcacttctttgaattagccgctcaggaatgtaaaaactacagagccctttctaattggttaactgttgagtttgccggacgttgtaaactcaagggtaagaatctcgctttctcaggtatcctgcccagtagtgtagctcagcttgtgaattttattgatcaaggcgtgattaccggaaagatcgctaaggatatcgcagacatgatgatggaatctcctgaaaagagtcctgagactatcctcaaagaaaatcctgaaatgttgcccatgacagatgaaagtgcgttggtggcgatcatttccgaggtgattaccgcaaatccgcagtctgtcgtagactacaaaagtggtaagaccaaggcgttaggatttttagttgggcaaattatgaaacgtacccagggcaaggcccctccaaatagggtaaatgaacttttgcttgtggaattaagtaaataa

SEQ ID NO: 19 - protein, 487 aa, Chlamydia psittaci
MSDVYADWESVIGLEVHVELNTKSKLFSCARNRFGDEPNTNISPVCTGMPGSLPVLNKEAVRKAVLFGCAVEGEVALLSRFDRKSYFYPDSPRNFQITQFEHPIVRGGHIKAIVHGEERHFELAQAHIEDDAGMLKHFGEFAGVDYNRAGVPLIEIVSKPCMFCADDAVAYATALVSLLDYIGISDCNMEEGSVRFDVNISVRPKGSEELRNKVEIKNMNSFAFMAQALEAERCRQIDAYLDNPNADPKTVIPGATYRWDPEKKKTVLMRLKERAEDYKYFIEPDLPVLQLTEAYIDEIRHTLPELPFNKYQRYLHEYALAEDIAAILISDKHSAHFFELAAQECKNYRALSNWLTVEFAGRCKLKGKNLAFSGILPSSVAQLVNFIDQGVITGKIAKDIADMMMESPEKSPETILKENPEMLPMTDESALVAIISEVITANPQSVVDYKSGKTKALGFLVGQIMKRTQGKAPPNRVNELLLVELSK

SEQ ID NO: 20 - DNA, 379 bp, Chlamydia psittaci
tatttagtgtcgaaaaacaacgccaacatttacgcaggttctctctattatcagcatatctcctattggagcgcttggcagaatctgctacaaaacactatcggtgcagaagctccgttagtccttaacgcacagttaacttattgtcatgcttcaaacgacatgaaaaccaacatgacgactacttacgctcctcgtaaaacaacgtatgcagaaatcaagggtgattggggtaacgattgtttcggagtcgagcttggtgcaactgtgcctatccaaacagaatcttctctcctattcgatatgtactcacctttcctgaagtttcaacttgtgcatacgcaccaagatgactttaaggaaaacaatagcgatcagg

SEQ ID NO: 21 - protein, 126 aa, Chlamydia psittaci
YLVSKNNANIYAGSLYYQHISYWSAWQNLLQNTIGAEAPLVLNAQLTYCHASNDMKTNMTTTYAPRKTTYAEIKGDWGNDCFGVELGATVPIQTESSLLFDMYSPFLKFQLVHTHQDDFKENNSDQ

SEQ ID NO: 22 - DNA, 2520 bp, Chlamydia psittaci
atgaaacatccagtctactggttcttaatatcctcgagcctatttgcctcgaattctttgagcttcgctaacgacgctcaaacagccttaactccctccgatagctataatggaaatgtgacctctgaggagttccaggtaaaagaaacttcatcaggaacaacgtatacttgtgaaggcaatgtgtgtatctcctttgcagggaaagattcaggtctaaagaaaagttgtttctcagctactgataaccttaccttcctaggaaacgggtatactctttgctttgataatattactactacagctagtaaccccggagccattaatgttcaaggtcaaggaaaaaccttaggcatctcaggattttctttattttcatgtgcttattgtcctccaggcacaactggttacggagctatacagactaaaggcaacacaactttaaaagataactctagtcttgtcttccataaaaactgctcaacagcagaaggtggggctatccagtgtaaaggaagcagtgatgctgaattaaaaatagaaaataatcagaatctggttttctcagaaaactcctccacttcaaaaggcggggctatttatgctgataaactcaccattgtctcaggtgggcctacattattttctaacaactctgtatccaacggttcatcccctaaaggcggagctattagcataaaagattcaagtggtgaatgtagcctaaccgctgatctcggagatattaccttcgatgggaacaaaatcatcaaaactagtggtggaagttctacagtaacaagaaattccatagatctcggcacagggaaatttacaaagctacgtgctaaagacggcttcggaattttcttctatgaccctattactgggggaggatctgatgaactaaacattaataaaaaagaaactgttgattatacaggaaagatcgtcttctcaggtgaaaaattatccgatgaagaaaaagcacgagcggaaaacctagcttctactttcaaccaacccatcacattatcagcaggatctcttgtacttaaagatggtgtatctgtaaccgcaaaacaagtaacgcaggaagcgggatctaccgttgtcatggatctagggaccacattacagacgccttcttcaggtggagaaaccatcaccctaactaatctagatattaacatcgcctcgttgggggggggggggggtacctctcctgctaaactcgcaacaaatacagcaagtcaagctataactattaacgctgtcaatctagtcgatgctgatggcaatgcttatgaagatcctattcttgctacgtctaaacctttcacagcaatagtagctacaactaacgctagtacagtcacacagcctacagataatctaacaaattatgtccctcctactcattacggttaccaaggaaattggacagtaacttgggacaccgaaacagctacaaaaacagccactctaacttgggaacaaactggctactcccctaacccagaacgtcaaggacctttagtcccgaatactctttggggtgcattctctgacctcagagctatacaaaacttaatggatattagcgtcaatggcgctgactaccatagaggtttttgggtatccggtctagctaacttcttacacaaaagtggctctgatactaaacgcaagttccgtcacaatagcgccggatacgctttaggcgtctacgcaaaaactccttctgatgatattttcagtgcggctttctgccaactcttcggaaaggacaaagactatttagtgtcgaaaaacaacgccaacatttacgcaggttctctctattatcagcatatctcctattggagcgcttggcagaatctgctacaaaacactatcggtgcagaagctccgttagtccttaacgcacagttaacttattgtcatgcttcaaacgacatgaaaaccaacatgacgactacttacgctcctcgtaaaacaacgtatgcagaaatcaagggtgattggggtaacgattgtttcggagtcgagcttggtgcaactgtgcctatccaaacagaatcttctctcctattcgatatgtactcacctttcctgaagtttcaacttgtgcatacgcaccaagatgactttaaggaaaacaatagcgatcagggaagatacttcgaaagcagcaatctcaccaacctttctctgcctatcggcatcaagtttgagagatttgctaacaacgatacagcttcttatcatgtcactgctgcttattctcctgatatcgtaagaagtaaccctgactgtactacttctctgttagtaagccccgactctgctgtctgggtaacgaaagccaacaaccttgcgcgaagcgccttcatgctacaagcaggaaactacttgtctttaagtcacaacatagaaatcttcagccagttcggtttcgagctcaggggatcttcacgaacctataacgtagatctcggatcgaagatccagttctaa

SEQ ID NO: 23 - protein, 839 aa, Chlamydia psittaci
MKHPVYWFLISSSLFASNSLSFANDAQTALTPSDSYNGNVTSEEFQVKETSSGTTYTCEGNVCISFAGKDSGLKKSCFSATDNLTFLGNGYTLCFDNITTTASNPGAINVQGQGKTLGISGFSLFSCAYCPPGTTGYGAIQTKGNTTLKDNSSLVFHKNCSTAEGGAIQCKGSSDAELKIENNQNLVFSENSSTSKGGAIYADKLTIVSGGPTLFSNNSVSNGSSPKGGAISIKDSSGECSLTADLGDITFDGNKIIKTSGGSSTVTRNSIDLGTGKFTKLRAKDGFGIFFYDPITGGGSDELNINKKETVDYTGKIVFSGEKLSDEEKARAENLASTFNQPITLSAGSLVLKDGVSVTAKQVTQEAGSTVVMDLGTTLQTPSSGGETITLTNLDINIASLGGGGGTSPAKLATNTASQAITINAVNLVDADGNAYEDPILATSKPFTAIVATTNASTVTQPTDNLTNYVPPTHYGYQGNWTVTWDTETATKTATLTWEQTGYSPNPERQGPLVPNTLWGAFSDLRAIQNLMDISVNGADYHRGFWVSGLANFLHKSGSDTKRKFRHNSAGYALGVYAKTPSDDIFSAAFCQLFGKDKDYLVSKNNANIYAGSLYYQHISYWSAWQNLLQNTIGAEAPLVLNAQLTYCHASNDMKTNMTTTYAPRKTTYAEIKGDWGNDCFGVELGATVPIQTESSLLFDMYSPFLKFQLVHTHQDDFKENNSDQGRYFESSNLTNLSLPIGIKFERFANNDTASYHVTAAYSPDIVRSNPDCTTSLLVSPDSAVWVTKANNLARSAFMLQAGNYLSLSHNIEIFSQFGFELRGSSRTYNVDLGSKIQF

SEQ ID NO: 24 - DNA, 1039 bp, Chlamydia psittaci
aaacgttttcatattaatggggttcctgaatggtctttatctacgccttattctcttgctatggggtataatatcttggctacgggagtgcagatggttaaagcctatgccattcttgccaacggtggttatgatgtgcgccctaccttgataaaaaaaatagtcactacttctggaaaagagtacgtgttgcatcctcaagttcgtggagaaagaattctttctcaggacattgtggatgaggtattgaaagctacgcgttttactacctatcctggaggaacgggatttcgggctgcgcctaaaaagcattccagtgcagggaaaacaggaacaacagaaaagctagttcatggaaagtatgataagcatcggcatatttcttcatttataggtatcacgccgatatacccttcggcaggggggagtgttcctttggtcatgcttgtctctatcagttatacgaccgacaacggtagtcaagtgtacgtcgttcaattgcgacatgagggtatcgaaatctgtcgtcaattcgtccatgttaacctaattgtgtggtcattatcgctttctttatactacttaccgtagttcctacggatactagcaaaaagttctgctctttgcgttgctctttgaacagcatactgtacttttaaaaagtctgctaaattttcccgttctccattcctatctgagaagtagagaagggctctatttaacacttcttctccagaagacacccaattgaccatcttacgggcaacggactcgtgttcttcttcttttttggtttgtaagttttgttgcgtatgctcagctatatcattcagatcaccattgattaaatcaatgatcacactgacagcttcaaaatgttcttgcgatagtttattttgatcttgttgtagagtggattgtgcatcccataaacgctcttttagattgtttatttgctctttcagctcttccgaatctaacgcctcttccagttcaggatcgataatgttagagtttctgtcttgcatcatcgccatag

SEQ ID NO: 25 - protein, 196 aa, Chlamydia psittaci
KRFHINGVPEWSLSTPYSLAMGYNILATGVQMVKAYAILANGGYDVRPTLIKKIVTTSGKEYVLHPQVRGERILSQDIVDEVLKATRFTTYPGGTGFRAAPKKHSSAGKTGTTEKLVHGKYDKHRHISSFIGITPIYPSAGGSVPLVMLVSISYTTDNGSQVYVVQLRHEGIEICRQFVHVNLIVWSLSLSLYYLP

SEQ ID NO: 26 - DNA, 1950 bp, Chlamydia psittaci
atgaatcaccgtaaatgcttaaccatgattacctatggagttctgctctcctattctttcctgatcatacggtattataaaattcagatttgtgaggagaaacgttgggcagcagaagctttaggacaacatgaatttcgagtaaaggacccttttcgtagggggacgtttttttctcagatgaatttacgtaagggagattcagagcaacgacaagctctggccgtggacattacgaaatttcatctttgtttagatgctgtagctgttcctgaagaacaccgtgatgtgattgctaagaaagtttttagtctcattggagaaggtgattatgacaaactccgtgcggagtttgataaaaaatctcgctatcgaaagttatttctttggttagatcgtgcggatcatgaccgcatcctgtcttggtggcgggggtacgcagcaaaatctaaaataccctcgaatgctttgtttttcatgaccgactatcaaagatcttatccctttggcaaacttttaggccaagttctacatactctgagagaagtcaaggatgagaaaacaggcaaagctttccctacaggaggtttagaagcctattttaaccacgtccttgaaggagagccaggagaacggaaattcctacgttctcctttaaatcgtttagatctagataaagtcacaaagattcctagggatggttcggatatttatctcacagtcaatccctgtatacagactatagcggaagaggaattagaaaaaggggtaaaggaagccaaagctaaaggtgggcgtctaattttaatgaatgcttatacaggcgagattcttgctttagcacagtatcctttctttaatccttcggaatacaaggaatttttcaatgataaggaaaaaatagagcacacaaaagtaacatcagtcagtgatgtgtttgaacccggctctatcatgaaacctctgactctggctatagcgttgctggccaacgaagagatggtgaaaagatcaggaaagcccttatttgatcctaatgaacctatagatgtaacccgcaggattttcccaggaagaaagcaatttccgcttaaggatatctcatcgaatcggcgtttaaatatgtacatggcgattcaaaagtcttcgaacgtttatgtagcgcaacttgctgatcttatagtgcaacatctagggaaccactggtatgaagacaagttattgttattaggatttggtaaaaagacggggatagaattgccaggggaagcgtcaggattggtaccttcacctaaacgttttcatattaatggggttcctgaatggtctttatctacgccttattctcttgctatggggtataatatcttggctacgggagtgcagatggttaaagcctatgccattcttgccaacggtggttatgatgtgcgccctaccttgataaaaaaaatagtcactacttctggaaaagagtacgtgttgcatcctcaagttcgtggagaaagaattctttctcaggacattgtggatgaggtattgaaagctacgcgttttactacctatcctggaggaacgggatttcgggctgcgcctaaaaagcattccagtgcagggaaaacaggaacaacagaaaagctagttcatggaaagtatgataagcatcggcatatttcttcatttataggtatcacgccgatatacccttcggcaggggggagtgttcctttggtcatgcttgtctctatagatgatcctgatcattgtgttcgcgaggatggaacaaagaactatatgggaggccgatgtgccgcccctgtatttggcagagttgcggatcgtgttttatcttatctaggagttcccgaagataaagaaaaatacagttatcagagtgaggtggctgctatgaaagctttgtatgaggaatggaatcgttcggggaaataa

SEQ ID NO: 27 - protein, 649 aa, Chlamydia psittaci
MNHRKCLTMITYGVLLSYSFLIIRYYKIQICEEKRWAAEALGQHEFRVKDPFRRGTFFSQMNLRKGDSEQRQALAVDITKFHLCLDAVAVPEEHRDVIAKKVFSLIGEGDYDKLRAEFDKKSRYRKLFLWLDRADHDRILSWWRGYAAKSKIPSNALFFMTDYQRSYPFGKLLGQVLHTLREVKDEKTGKAFPTGGLEAYFNHVLEGEPGERKFLRSPLNRLDLDKVTKIPRDGSDIYLTVNPCIQTIAEEELEKGVKEAKAKGGRLILMNAYTGEILALAQYPFFNPSEYKEFFNDKEKIEHTKVTSVSDVFEPGSIMKPLTLAIALLANEEMVKRSGKPLFDPNEPIDVTRRIFPGRKQFPLKDISSNRRLNMYMAIQKSSNVYVAQLADLIVQHLGNHWYEDKLLLLGFGKKTGIELPGEASGLVPSPKRFHINGVPEWSLSTPYSLAMGYNILATGVQMVKAYAILANGGYDVRPTLIKKIVTTSGKEYVLHPQVRGERILSQDIVDEVLKATRFTTYPGGTGFRAAPKKHSSAGKTGTTEKLVHGKYDKHRHISSFIGITPIYPSAGGSVPLVMLVSIDDPDHCVREDGTKNYMGGRCAAPVFGRVADRVLSYLGVPEDKEKYSYQSEVAAMKALYEEWNRSGK

SEQ ID NO: 28 - DNA, 960 bp, Chlamydia psittaci
atgttcaataagctcattgaaacagcacagaaacgggtggaagcaagaaactatactattcgaaagcatactcttgagtatgacgatgttatgaataggcaaaggcagacgatctatgcttttcgtaatgacgttatccgctctgaagatatctttggtttagctaaggaagcaatatctcatgttgcattaatgatcgcttcgttgatagtgagccgtgatcatcctacagggaattctcttcctaggctggaagaatggatgaactattctttcccactgcaattgaatattgaagaattgaaaagattgaagtctatagatgccattgccgaacgggttgctgatgatctcatagaagttttccagaataagtttgcttctatggtgcaggaaattaccgaagcagccggagaaaaagtcgatgctaatggtgtctgtaaagatgttattcgctcggtcatgattatgcatatcgatgagcagtggaaaattcatcttgtagatatggatttattacgtagtgaagtaggtttacgtactgtcggtcagaaagaccctcttatcgaatttaaacatgagtcgttcttactattcgaaagtcttattcgcgatattcgtattgctattgtaaagcatttgttccgtttagagttgacgatgactagagaacagcggcctcaaaatgtcgtgcctgttgttgccacatctttccaaaataatgaaaatttcggtcctttggaactcacagttatcagtgattctgacgatgaataaaaagagctttagggctgggctagcttccagccttttcccttacgttattgatttatagttttaaataaatacggaccactcagaccaggattgtgtgtcgtggtggcgtatccaaaatgttctgtgattatcctcaatcagaaattgtacatgatgatcgcgattgcgtgttgtcatgcaaat

SEQ ID NO: 29 - protein, 258 aa, Chlamydia psittaci
MFNKLIETAQKRVEARNYTIRKHTLEYDDVMNRQRQTIYAFRNDVIRSEDIFGLAKEAISHVALMIASLIVSRDHPTGNSLPRLEEWMNYSFPLQLNIEELKRLKSIDAIAERVADDLIEVFQNKFASMVQEITEAAGEKVDANGVCKDVIRSVMIMHIDEQWKIHLVDMDLLRSEVGLRTVGQKDPLIEFKHESFLLFESLIRDIRIAIVKHLFRLELTMTREQRPQNVVPVVATSFQNNENFGPLELTVISDSDDE

SEQ ID NO: 30 - DNA, 697 bp, Chlamydia psittaci
gggtttgattatctcagagataattctattgcaacttctgtggatgagcaggtgggacgtgggttttattttgctattatcgatgaagtcgactcgattttaattgatgaagccagaactcctttaattatttctggtcctggggaaaaacataatcctgtgtatttcgaactcaaagataaagtggctgacctcgttcagttacaaagggagttatgtaaccagttagctcttgaagctagacggggactagaattgttcttggatatggatattcttcctaaggataaaaaagttatcgaagctatctccgaattttgccgtagcttatggttagttagtaagggaatgcctttaaatcgtgttttgcgtagagtgcgcgaacacccagatttgcgagccatgatagataaatgggatacttattatcatgctgagcaaaataaagaagagagtatagagaagctatctcagctgtatatcattgttgatgaacataataacgattttgaattgacagatcgtggcatgcaacaatgggtggataaggctggaggttctgctgaagattttgtcatgatggacatggggcatgaatatgctcttatagatggtgacgataccttatcaccgacagagaaaatcaatagaaaaatagctatttccgaagaagatacgaggagaaaagctcgagctc

SEQ ID NO: 31 - protein, 232 aa, Chlamydia psittaci
GFDYLRDNSIATSVDEQVGRGFYFAIIDEVDSILIDEARTPLIISGPGEKHNPVYFELKDKVADLVQLQRELCNQLALEARRGLELFLDMDILPKDKKVIEAISEFCRSLWLVSKGMPLNRVLRRVREHPDLRAMIDKWDTYYHAEQNKEESIEKLSQLYIIVDEHNNDFELTDRGMQQWVDKAGGSAEDFVMMDMGHEYALIDGDDTLSPTEKINRKIAISEEDTRRKARA

SEQ ID NO: 32 - DNA, 2910 bp, Chlamydia psittaci
atgttagattttcttaaacgtttctttggatcttctcaagagcgcaccttaaaaaaatttcaaaaacttgtggataaggtcaacctctatgatgagatgctagctcctttgtctgatgaggagttacgtaataaaacagcagagttaaaaaagcgttatcaggacggcgaatccttagatgatatgcttcccgaggcttatgccgtagtgaaaaatgtatgcaggcgtttaacaggaactcctgtagaagtgtcgggttatcatcaaaattgggacatggttccctatgatgtgcaggttctcggtgctatagctatgcataagggctttataaccgagatgcagacaggagaggggaaaactctcaccgctgttatgcctctatatttaaatgcattgacaggcaagcctgtgcatttagtcacagtgaatgattatctcgctcaaagggattgtgagtgggtcggctctatattgcgttggttaggtttaactaccggagtattgatatcaggatcgcctttagaaaaaagaaaagacatttatcgttgtgacgttgtctacggtacagcatcagagttcgggtttgattatctcagagataattctattgcaacttctgtggatgagcaggtgggacgtgggttttattttgctattatcgatgaagtcgactcgattttaattgatgaagccagaactcctttaattatttctggtcctggggaaaaacataatcctgtgtatttcgaactcaaagataaagtggctgacctcgttcagttacaaagggagttatgtaaccagttagctcttgaagctagacggggactagaattgttcttggatatggatattcttcctaaggataaaaaagttatcgaagctatctccgaattttgccgtagcttatggttagttagtaagggaatgcctttaaatcgtgttttgcgtagagtgcgcgaacacccagatttgcgagccatgatagataaatgggatacttattatcatgctgagcaaaataaagaagagagtatagagaagctatctcagctgtatatcattgttgatgaacataataacgattttgaattgacagatcgtggcatgcaacaatgggtggataaggctggaggttctgctgaagattttgtcatgatggacatggggcatgaatatgctcttatagatggtgacgataccttatcaccgacagagaaaatcaatagaaaaatagctatttccgaagaagatacgaggagaaaagctcgagctcatggcttgcgccaactattaagagcgcatcttcttatggaacgcgatgtggattatattgttcgtaatgatcaaattgtcatcattgacgaacatacgggccgcccgcaaccaggtcgtcgtttttccgaaggactgcatcaagccatagaagcaaaagaacatgtcactatccgtaaggaatcacaaacgtttgctacagttaccttacagaatttcttccgtctgtatgaaaaactcgcaggtatgacgggaacagcaattacggaatctaaagagtttaaagagatttataatctttatgtattgcaggtgcccacgtttaaagaatgtttgcgtgtagatcacaatgacgaattttatatgacagagcgtgaaaagtaccacgcgattgttaaggaaattgcccgtatacatgccgtagggaacccgattctcataggaacggagtctgtagaggtttctgagaaactttctcgtattttgagacaaaatcgcatagaacatacagtgttaaatgcgaaaaatcatgctcaagaagcagagatcattgcagcagcaggaaagctgggagctgtgactgtagctaccaatatggctggccgtggtacagatattaagctggatgaagaagctgtagttgttggaggtctccatgttattggtacgagtcggcaccaatcacgccgtatagataggcagttgcgcgggcgttgcgcacgtttaggagatcctggttcggcgaaatttttcctatcttttgaagatcgcctgatgcgcttatttgcatcgcccaagttaaatgccttgattcgtcatttccgtcctcctgaaggagaggctatgtcggatcctatgttcaataagctcattgaaacagcacagaaacgggtggaagcaagaaactatactattcgaaagcatactcttgagtatgacgatgttatgaataggcaaaggcagacgatctatgcttttcgtaatgacgttatccgctctgaagatatctttggtttagctaaggaagcaatatctcatgttgcattaatgatcgcttcgttgatagtgagccgtgatcatcctacagggaattctcttcctaggctggaagaatggatgaactattctttcccactgcaattgaatattgaagaattgaaaagattgaagtctatagatgccattgccgaacgggttgctgatgatctcatagaagttttccagaataagtttgcttctatggtgcaggaaattaccgaagcagccggagaaaaagtcgatgctaatggtgtctgtaaagatgttattcgctcggtcatgattatgcatatcgatgagcagtggaaaattcatcttgtagatatggatttattacgtagtgaagtaggtttacgtactgtcggtcagaaagaccctcttatcgaatttaaacatgagtcgttcttactattcgaaagtcttattcgcgatattcgtattgctattgtaaagcatttgttccgtttagagttgacgatgactagagaacagcggcctcaaaatgtcgtgcctgttgttgccacatctttccaaaataatgaaaatttcggtcctttggaactcacagttatcagtgattctgacgatgaataa

SEQ ID NO: 33 - protein, 969 aa, Chlamydia psittaci
MLDFLKRFFGSSQERTLKKFQKLVDKVNLYDEMLAPLSDEELRNKTAELKKRYQDGESLDDMLPEAYAVVKNVCRRLTGTPVEVSGYHQNWDMVPYDVQVLGAIAMHKGFITEMQTGEGKTLTAVMPLYLNALTGKPVHLVTVNDYLAQRDCEWVGSILRWLGLTTGVLISGSPLEKRKDIYRCDVVYGTASEFGFDYLRDNSIATSVDEQVGRGFYFAIIDEVDSILIDEARTPLIISGPGEKHNPVYFELKDKVADLVQLQRELCNQLALEARRGLELFLDMDILPKDKKVIEAISEFCRSLWLVSKGMPLNRVLRRVREHPDLRAMIDKWDTYYHAEQNKEESIEKLSQLYIIVDEHNNDFELTDRGMQQWVDKAGGSAEDFVMMDMGHEYALIDGDDTLSPTEKINRKIAISEEDTRRKARAHGLRQLLRAHLLMERDVDYIVRNDQIVIIDEHTGRPQPGRRFSEGLHQAIEAKEHVTIRKESQTFATVTLQNFFRLYEKLAGMTGTAITESKEFKEIYNLYVLQVPTFKECLRVDHNDEFYMTEREKYHAIVKEIARIHAVGNPILIGTESVEVSEKLSRILRQNRIEHTVLNAKNHAQEAEIIAAAGKLGAVTVATNMAGRGTDIKLDEEAVVVGGLHVIGTSRHQSRRIDRQLRGRCARLGDPGSAKFFLSFEDRLMRLFASPKLNALIRHFRPPEGEAMSDPMFNKLIETAQKRVEARNYTIRKHTLEYDDVMNRQRQTIYAFRNDVIRSEDIFGLAKEAISHVALMIASLIVSRDHPTGNSLPRLEEWMNYSFPLQLNIEELKRLKSIDAIAERVADDLIEVFQNKFASMVQEITEAAGEKVDANGVCKDVIRSVMIMHIDEQWKIHLVDMDLLRSEVGLRTVGQKDPLIEFKHESFLLFESLIRDIRIAIVKHLFRLELTMTREQRPQNVVPVVATSFQNNENFGPLELTVISDSDDE

SEQ ID NO: 34 - DNA, 577 bp, Chlamydia psittaci
gttgatgctgcagttattccagggaacttcgccattgcagggggaatctgtccgtataaaaacagtctatacctagaagatgtccgtacttcccaatacaccaatgtcgttgtcatacgtgctgaagatatggaagactcgagaatgcataaactaaaacagctattgcaaagcagttctgtgcaggatttctttaatacgaaatataaggggatctttttatcgcagtaacacatctggatggcttagggaagagttgagccaccccgttctccgtaggtttaaggcatattgggaaacgattttcttgaattttttgaaaaactttgactgtttttcttttgattattcgaagcagatgtatgtcgagtatggcggttttagggcccagaggtcctttcagttctccttttacatgttctctatacccaacccacctaaaaatgcacttgctaggttccattcctatagttggcatatacattggagcgaagcggatagccgccgttgctcaatatcatagaatgtgtagagcgaatacaggagtgtctcaggtgattattcaggattcaggatt

SEQ ID NO: 35 - protein, 76 aa, Chlamydia psittaci
VDAAVIPGNFAIAGGICPYKNSLYLEDVRTSQYTNVVVIRAEDMEDSRMHKLKQLLQSSSVQDFFNTKYKGIFLSQ

SEQ ID NO: 36 - DNA, 804 bp, Chlamydia psittaci
atgaaaaaaatcacaatactctcgttacttgctttagccatctctttaacaggttgttgcaagaattcagaaggagtcttgcggattgcggcgagtcccacgccacatgcagagcttctttatagtttagaaaaggaggctcaatcccttggattgcaattgaaaatacttcccatagatgattaccgtgtacctaaccgtttgcttttagataagcaaatagaggcaaattatttccaacatgaagatttcttaaaagatgagtgtgctcggtaccaatgcgaaggaaaacttgcgattttggctaaggtacatttagaacctatgggtttatattctaataaaacccagtctctcgaagagcttaaagtcaaggaacagctacgtatagcggttcctatagatagaacaaacgaacaacgtgcgctagacttattgcgagactgcaatttgattagttacaaagaagcttctcatctagatatcaccgcaaaagatgtctttggttgtggagggaaaaaggtaacgattatagagatggcagcacctttattagtatcttctttaccagacgttgatgctgcagttattccagggaacttcgccattgcagggggaatctgtccgtataaaaacagtctatacctagaagatgtccgtacttcccaatacaccaatgtcgttgtcatacgtgctgaagatatggaagactcgagaatgcataaactaaaacagctattgcaaagcagttctgtgcaggatttctttaatacgaaatataaggggatctttttatcgcagtaa

SEQ ID NO: 37 - protein, 267 aa, Chlamydia psittaci
MKKITILSLLALAISLTGCCKNSEGVLRIAASPTPHAELLYSLEKEAQSLGLQLKILPIDDYRVPNRLLLDKQIEANYFQHEDFLKDECARYQCEGKLAILAKVHLEPMGLYSNKTQSLEELKVKEQLRIAVPIDRTNEQRALDLLRDCNLISYKEASHLDITAKDVFGCGGKKVTIIEMAAPLLVSSLPDVDAAVIPGNFAIAGGICPYKNSLYLEDVRTSQYTNVVVIRAEDMEDSRMHKLKQLLQSSSVQDFFNTKYKGIFLSQ

SEQ ID NO: 38 - DNA, 402 bp, Chlamydia psittaci
catgtattttacgcaaaaaataaacggtataactcctgcttacaagccgcgctataccacaataatatcccgacaaccgtgtacacaaaccttattgatatcgtgaagaaaaattcttcactaatcacgaagtacttttccatcaaacaacgatgcttaaatctaaaagatttccatttttatgatgtttatgctcccctaagtcagtccaaagagaaaaaatatacgttccaagaagctgtggatcttatctatactagcctttctcctctaggaacggaatacattgataccttaaaacaggggttaacaactcaaggctgggtagataaatacgaaaatcttaataaacgctccggagcctattcttcgggatgttacgatagccacccttatgtcctc

SEQ ID NO: 39 - protein, 134 aa, Chlamydia psittaci
HVFYAKNKRYNSCLQAALYHNNIPTTVYTNLIDIVKKNSSLITKYFSIKQRCLNLKDFHFYDVYAPLSQSKEKKYTFQEAVDLIYTSLSPLGTEYIDTLKQGLTTQGWVDKYENLNKRSGAYSSGCYDSHPYVL

SEQ ID NO: 40 - DNA, 1827 bp, Chlamydia psittaci
atgagcgtagaattcaacaagcaacaagtccgtccaagaagtgaaatttcccctcaagattgttgggatatcacccccttatatctaaatagaaaagcatggaaagcagatcttgattctttcggattaaaaacagacggctcacctacgtggcccgctcttcaagcaacgcaataccaactggacaactcagaatctctactatccttattaactactctcttctctattgagagaaaattaaacaaactctacgtttacgctcatctgactcatgatcaggatattacaaatcaagaaggcatcgcagatcttaaatctatcacgcatctacataccttatttgccgaagaaacctcttgggtacaacccgctttaaccagcctatcggaatctctcattgctcagcacctatcagctccctgtttagctccttatagattctatttagagaaaatctttagactatctatacacacaggcactcctggagaagaaaaaattctcgcttccgcctttactcctcttgaagtagccagtaaggcattttcttctttaagtgactctgaaattccctttgggcaagctacagactcagaaggaaactctcacccgctttctcatgcactggcttcattgtatatgcaatccacagatcgggaattacgaaaaacatcctacctagcacaatgtgaaagatatcatagttaccgacatacctttgctaacttactcaatgggaaaatccaagcccatgtattttacgcaaaaaataaacggtataactcctgcttacaagccgcgctataccacaataatatcccgacaaccgtgtacacaaaccttattgatatcgtgaagaaaaattcttcactaatcacgaagtacttttccatcaaacaacgatgcttaaatctaaaagatttccatttttatgatgtttatgctcccctaagtcagtccaaagagaaaaaatatacgttccaagaagctgtggatcttatctatactagcctttctcctctaggaacggaatacattgataccttaaaacaggggttaacaactcaaggctgggtagataaatacgaaaatcttaataaacgctccggagcctattcttcgggatgttacgatagccacccttatgtcctcctaaactatacaggcaccctgtatgatgtatccgtcattgcccacgaaggcggacacagtatgcactcgtattttagtaggaagcatcaacctttccatgacgctcaatatcctattttccttgctgaaattgcttctaccttaaatgaaatgcttcttatggattccatgctgaaggagagcgactcaaaagaagagaaaatcaccattctgacacgatgtttggataccatcttctctacactattccgtcaggtattattcgcctcttttgaatacgatattcatcacgcagcagaacatggggttcctctaactgaagaatacctatcctcaacttacaagaatttacaaaatgagttttacggagaaattatcacatttgatgtcctgtcgaacatagaatgggcaagaattcctcatttctattacaatttctacgtataccaatatgcaacgggcattatagccgccctgtgctttttagaaaaaattcttaacaacgaagataacgctcttaactcctatctcaactttttaaaaagtggtggatcagatttccccttagaaatcttaaaaaaatcaggattagatatgggcacagttgagccaatccaaaaagctttttgctttatcgagaaaaaaatccaggagctatcatctttaatttga

SEQ ID NO: 41 - protein, 608 aa, Chlamydia psittaci
MSVEFNKQQVRPRSEISPQDCWDITPLYLNRKAWKADLDSFGLKTDGSPTWPALQATQYQLDNSESLLSLLTTLFSIERKLNKLYVYAHLTHDQDITNQEGIADLKSITHLHTLFAEETSWVQPALTSLSESLIAQHLSAPCLAPYRFYLEKIFRLSIHTGTPGEEKILASAFTPLEVASKAFSSLSDSEIPFGQATDSEGNSHPLSHALASLYMQSTDRELRKTSYLAQCERYHSYRHTFANLLNGKIQAHVFYAKNKRYNSCLQAALYHNNIPTTVYTNLIDIVKKNSSLITKYFSIKQRCLNLKDFHFYDVYAPLSQSKEKKYTFQEAVDLIYTSLSPLGTEYIDTLKQGLTTQGWVDKYENLNKRSGAYSSGCYDSHPYVLLNYTGTLYDVSVIAHEGGHSMHSYFSRKHQPFHDAQYPIFLAEIASTLNEMLLMDSMLKESDSKEEKITILTRCLDTIFSTLFRQVLFASFEYDIHHAAEHGVPLTEEYLSSTYKNLQNEFYGEIITFDVLSNIEWARIPHFYYNFYVYQYATGIIAALCFLEKILNNEDNALNSYLNFLKSGGSDFPLEILKKSGLDMGTVEPIQKAFCFIEKKIQELSSLI

SEQ ID NO: 42 - DNA, 1517 bp, Chlamydia psittaci
gcgttagattcggaagagctgaaagagcaaataaacaatctaaaagagcgtttatgggatgcacaatccactctacaacaagatcaaaataaactatcgcaagaacattttgaagctgtcagtgtgatcattgatttaatcaatggtgatctgaatgatatagctgagcatacgcaacaaaacttacaaaccaaaaaagaagaagaacacgagtccgttgcccgtaagatggtcaattgggtgtcttctggagaagaagtgttaaatagagcccttctctacttctcagataggaatggagaacgggaaaatttagcagactttttaaaagtacagtatgctgttcaaagagcaacgcaaagagcagaactttttgctagtatcgtaggaactacggtaagtagtataaagacgataatgaccacacaattaggttaacatggacgaattgacgacagatttcgataccctcatgtcgcaattgaacgacgtacacttgactaccgttgtcggtcgtataactgaagtcgtcggtatgttaattaaagctgtcgttcccaatgtacgcgttggggaggtatgcttagttaaacgttatggtatggagccgctcgtgaccgaagtcgtcggcttcacacaaaatttcgcttttttatcgccactaggagaacttactggagtcagcccttcttcagaggttattcccacaggtctgcctttgtatatccgtgcaggtaacggtcttttaggtcgtgtattgaatggtctgggagaacctatcgactccgagatcaaaggacctttggttgatgttaacgaaacctaccctgtgtttcgcgctccaccagatccattgcatagagaaaaattaagaacaattttatccaccggcgtgcggtgtatcgacggtatgctcacagtcgccagaggccagcgtataggcatttttgctggagctggggtgggtaaatcgtctctcttgggaatgatcgctagaaacgcggaagaagccgatgtcaatgtgattgctctcatcggagagcggggccgagaggttcgtgaatttatcgagggcgatctcggagaagaaggaatgaaacgttcggtgatcgtcgtctctacttcagatcaatcctcacagttgcgattaaatgctgcttacgtaggcaccgctatagcagagtattttcgtgatcagggcaaaaccgtagttttgatgatggattctgtcacccgatttgcccgagccctaagagaagtcggtctagctgccggagaaccgccagctcgaggaggatacacaccttctgtattctcaactttgcctaggttattagaacgttccggagcttcggataaaggaacaatcacagccttttacacagtacttgttgccggggatgatatgaatgaaccggtcgctgatgaagttaaatcgattcttgatggtcacgttgtcttgtctaacgctttagctcaggcataccattatcctgctattgatgtcttagcatcta

SEQ ID NO: 43 - protein, 145 aa, Chlamydia psittaci
ALDSEELKEQINNLKERLWDAQSTLQQDQNKLSQEHFEAVSVIIDLINGDLNDIAEHTQQNLQTKKEEEHESVARKMVNWVSSGEEVLNRALLYFSDRNGERENLADFLKVQYAVQRATQRAELFASIVGTTVSSIKTIMTTQLG

SEQ ID NO: 44 - DNA, 669 bp, Chlamydia psittaci
atggtagatcctttgaagcttttcccaaagctagactccgagaaagaaacagcttctatacagaagcctttaggaactcctttagccagtgagttacataaggaagttcctgcattttctttagggacggcagcagactccttgaataaaaatatagaggatgtcaagcctaaccctatggcgatgatgcaagacagaaactctaacattatcgatcctgaactggaagaggcgttagattcggaagagctgaaagagcaaataaacaatctaaaagagcgtttatgggatgcacaatccactctacaacaagatcaaaataaactatcgcaagaacattttgaagctgtcagtgtgatcattgatttaatcaatggtgatctgaatgatatagctgagcatacgcaacaaaacttacaaaccaaaaaagaagaagaacacgagtccgttgcccgtaagatggtcaattgggtgtcttctggagaagaagtgttaaatagagcccttctctacttctcagataggaatggagaacgggaaaatttagcagactttttaaaagtacagtatgctgttcaaagagcaacgcaaagagcagaactttttgctagtatcgtaggaactacggtaagtagtataaagacgataatgaccacacaattaggttaa

SEQ ID NO: 45 - protein, 222 aa, Chlamydia psittaci
MVDPLKLFPKLDSEKETASIQKPLGTPLASELHKEVPAFSLGTAADSLNKNIEDVKPNPMAMMQDRNSNIIDPELEEALDSEELKEQINNLKERLWDAQSTLQQDQNKLSQEHFEAVSVIIDLINGDLNDIAEHTQQNLQTKKEEEHESVARKMVNWVSSGEEVLNRALLYFSDRNGERENLADFLKVQYAVQRATQRAELFASIVGTTVSSIKTIMTTQLG

SEQ ID NO: 46 - DNA, 1329 bp, Chlamydia psittaci
atggacgaattgacgacagatttcgataccctcatgtcgcaattgaacgacgtacacttgactaccgttgtcggtcgtataactgaagtcgtcggtatgttaattaaagctgtcgttcccaatgtacgcgttggggaggtatgcttagttaaacgttatggtatggagccgctcgtgaccgaagtcgtcggcttcacacaaaatttcgcttttttatcgccactaggagaacttactggagtcagcccttcttcagaggttattcccacaggtctgcctttgtatatccgtgcaggtaacggtcttttaggtcgtgtattgaatggtctgggagaacctatcgactccgagatcaaaggacctttggttgatgttaacgaaacctaccctgtgtttcgcgctccaccagatccattgcatagagaaaaattaagaacaattttatccaccggcgtgcggtgtatcgacggtatgctcacagtcgccagaggccagcgtataggcatttttgctggagctggggtgggtaaatcgtctctcttgggaatgatcgctagaaacgcggaagaagccgatgtcaatgtgattgctctcatcggagagcggggccgagaggttcgtgaatttatcgagggcgatctcggagaagaaggaatgaaacgttcggtgatcgtcgtctctacttcagatcaatcctcacagttgcgattaaatgctgcttacgtaggcaccgctatagcagagtattttcgtgatcagggcaaaaccgtagttttgatgatggattctgtcacccgatttgcccgagccctaagagaagtcggtctagctgccggagaaccgccagctcgaggaggatacacaccttctgtattctcaactttgcctaggttattagaacgttccggagcttcggataaaggaacaatcacagccttttacacagtacttgttgccggggatgatatgaatgaaccggtcgctgatgaagttaaatcgattcttgatggtcacgttgtcttgtctaacgctttagctcaggcataccattatcctgctattgatgtcttagcatctatcagccgattgctgacagcaattgttcctgaggaacaacgacgcatcataggaaaagcccgagaggtgctggcaaaatacaaagcaaacgaaatgcttatacgtattggagaatatcgccgagggtccgatcgtgaagtggattttgctatagatcacattgataaattgaacagattcttaaagcaagatattcatgaaaaaacaaattacgaggaagcctcgcaacagcttcgggctattttccgataa

SEQ ID NO: 47 - protein, 442 aa, Chlamydia psittaci
MDELTTDFDTLMSQLNDVHLTTVVGRITEVVGMLIKAVVPNVRVGEVCLVKRYGMEPLVTEVVGFTQNFAFLSPLGELTGVSPSSEVIPTGLPLYIRAGNGLLGRVLNGLGEPIDSEIKGPLVDVNETYPVFRAPPDPLHREKLRTILSTGVRCIDGMLTVARGQRIGIFAGAGVGKSSLLGMIARNAEEADVNVIALIGERGREVREFIEGDLGEEGMKRSVIVVSTSDQSSQLRLNAAYVGTAIAEYFRDQGKTVVLMMDSVTRFARALREVGLAAGEPPARGGYTPSVFSTLPRLLERSGASDKGTITAFYTVLVAGDDMNEPVADEVKSILDGHVVLSNALAQAYHYPAIDVLASISRLLTAIVPEEQRRIIGKAREVLAKYKANEMLIRIGEYRRGSDREVDFAIDHIDKLNRFLKQDIHEKTNYEEASQQLRAIFR

SEQ ID NO: 48 - DNA, 477 bp, Chlamydia psittaci
cttcttgcagatgccgactctgtcaaccttgcaactggattcaacggctccactagtgaaactttcaatgttaaacaaacagataatgctgacgggacaacatatattctaggcagcgcgatcacctttgaacacataaatcaattaaaaccagcaaacactagctgttttgctaatacagctggagatctaacgtttactgggaatcgacgacttctctatttcaataatatttcatcaacagcgaaaggtgccgctatcagcacaactgcggatggtaagacactcacaatatccggggctctacaactgattttctacatgtcgccaagattggccacgggaaatggcgtcatttattctaatagctctgtactcatcgagaacaattctcaaggtagctcgggactgaataagtctgcagggaaaggcgtctttatttgttgtgagaaaagtacggatgtgggagctacatca

SEQ ID NO: 49 - protein, 159 aa, Chlamydia psittaci
LLADADSVNLATGFNGSTSETFNVKQTDNADGTTYILGSAITFEHINQLKPANTSCFANTAGDLTFTGNRRLLYFNNISSTAKGAAISTTADGKTLTISGALQLIFYMSPRLATGNGVIYSNSSVLIENNSQGSSGLNKSAGKGVFICCEKSTDVGATS

SEQ ID NO: 50 - DNA, 591 bp, Chlamydia psittaci
acctttgaacacataaatcaattaaaaccagcaaacactagctgttttgctaatacagctggagatctaacgtttactgggaatcgacgacttctctatttcaataatatttcatcaacagcgaaaggtgccgctatcagcacaactgcggatggtaagacactcacaatatccggggctctacaactgattttctacatgtcgccaagattggccacgggaaatggcgtcatttattctaatagctctgtactcatcgagaacaattctcaaggtagctcgggactgaataagtctgcagggaaaggcgtctttatttgttgtgagaaaagtacggatgtgggagctacatcaccgacattaatcatacggaataacggagagtttcttactgtaggtaatgcagctactagctctggaggagcgatttatgcggagaaaatgatcttatcctcaggaggatatacaaaatttcaatccaatgttagctatgatcaaggtggggccattgccattgctcctaatggagaaattagtctctccgcggataaaggaaatatcgtctttgaaagaaaccttaaaattgccaac

SEQ ID NO: 51 - protein, 197 aa, Chlamydia psittaci
TFEHINQLKPANTSCFANTAGDLTFTGNRRLLYFNNISSTAKGAAISTTADGKTLTISGALQLIFYMSPRLATGNGVIYSNSSVLIENNSQGSSGLNKSAGKGVFICCEKSTDVGATSPTLIIRNNGEFLTVGNAATSSGGAIYAEKMILSSGGYTKFQSNVSYDQGGAIAIAPNGEISLSADKGNIVFERNLKIAN

SEQ ID NO: 52 - DNA, 2040 bp, Chlamydia psittaci
atgcagggaatactaatgaaaaactctatttatggggttttactgttttcctcttttgccttatccactgctaccaaacttcttgcagatgccgactctgtcaaccttgcaactggattcaacggctccactagtgaaactttcaatgttaaacaaacagataatgctgacgggacaacatatattctaggcagcgcgatcacctttgaacacataaatcaattaaaaccagcaaacactagctgttttgctaatacagctggagatctaacgtttactgggaatcgacgacttctctatttcaataatatttcatcaacagcgaaaggtgccgctatcagcacaactgcggatggtaagacactcacaatatccggggctctacaactgattttctacatgtcgccaagattggccacgggaaatggcgtcatttattctaatagctctgtactcatcgagaacaattctcaaggtagctcgggactgaataagtctgcagggaaaggcgtctttatttgttgtgagaaaagtacggatgtgggagctacatcaccgacattaatcatacggaataacggagagtttcttactgtaggtaatgcagctactagctctggaggagcgatttatgcggagaaaatgatcttatcctcaggaggatatacaaaatttcaatccaatgttagctatgatcaaggtggggccattgccattgctcctaatggagaaattagtctctccgcggataaaggaaatatcgtctttgaaagaaaccttaaaattgccaacaaacaaaatactcccaatgccattcacctaggagacaatgcgaaatttcttcaattacgtgctgctaacaacaaagccatatttttttatgacccgattacaaccacgggatctgtggcagatcggctaattattaataactcgcaaggagaagcctcgacttacgatggggcgattgtattttctagtctcaacttattcactcattcccctgaatgtaaactctcttcattttctcaaggtcttactttagcggcaggatcattagttttagaagagggggtatgtgtacaagctccgtcttttgatcaacgtgctcactcccaactattcatgaatcctgggacgaagttacaagctacccagaacatctcggtaaagaatctccatctcaatcttaatagaatagcagaagagccggcgtatatcaccacaacagacgatgcttctagtgtggacatttgcggacctgtagttatgcatatagatgatgagatcttctataatcagacagtattagcaaatgagttgtctgtagagtgtttaaatctcagttctccacatctcgataatatcactattgatgacgttcccgcagtgcctatcatgacgttagaatcgcatcgtggttatcaaggtacatgggaaatctcttggaaagagcaacctaaacttacctttgggaaggcgactatcgcgcctaataagcagatgcaccttatttggaaaccttctggttacgttcctttctcagggggaactggagagtttacgacatctttagtgcctaatagcttatggaatctctttttagatacacgtttttctcaacaagcgattgagaaacatgctgtatcttcaggtaacggtatatggatttcctccatgaccaattcttttcttcaaggttctacgaacaacaaccacggctttcgtcataagagttcaggatataccgcagggggaaaaatacaaacacttcaagatgatatctttagtgtcagtttttctcagctatttgggagatctaaggattttggatctgccacatctaaggatacattcctatcgggctctatctatgctcagcattcgagacgcttacttcctataatgagattccttgcaggaacatcaacatatagaccgcgactcttactgagtattcccaagaatcttcctatcaattttgatgttcttgtgagttacagctatgacagtaaccacatgaaagtacaaaaattctaa

SEQ ID NO: 53 - protein, 679 aa, Chlamydia psittaci
MQGILMKNSIYGVLLFSSFALSTATKLLADADSVNLATGFNGSTSETFNVKQTDNADGTTYILGSAITFEHINQLKPANTSCFANTAGDLTFTGNRRLLYFNNISSTAKGAAISTTADGKTLTISGALQLIFYMSPRLATGNGVIYSNSSVLIENNSQGSSGLNKSAGKGVFICCEKSTDVGATSPTLIIRNNGEFLTVGNAATSSGGAIYAEKMILSSGGYTKFQSNVSYDQGGAIAIAPNGEISLSADKGNIVFERNLKIANKQNTPNAIHLGDNAKFLQLRAANNKAIFFYDPITTTGSVADRLIINNSQGEASTYDGAIVFSSLNLFTHSPECKLSSFSQGLTLAAGSLVLEEGVCVQAPSFDQRAHSQLFMNPGTKLQATQNISVKNLHLNLNRIAEEPAYITTTDDASSVDICGPVVMHIDDEIFYNQTVLANELSVECLNLSSPHLDNITIDDVPAVPIMTLESHRGYQGTWEISWKEQPKLTFGKATIAPNKQMHLIWKPSGYVPFSGGTGEFTTSLVPNSLWNLFLDTRFSQQAIEKHAVSSGNGIWISSMTNSFLQGSTNNNHGFRHKSSGYTAGGKIQTLQDDIFSVSFSQLFGRSKDFGSATSKDTFLSGSIYAQHSRRLLPIMRFLAGTSTYRPRLLLSIPKNLPINFDVLVSYSYDSNHMKVQKF

SEQ ID NO: 54 - DNA, 487 bp, Chlamydia psittaci
acctcgagagaggattctcttagtgtggctttctgtcagttatttgcaaaagataaagactaccttgtaagcaagaacgccgcaaacgtctatgcgggttctgtatattatcagcatgtgagcaagtttgatgatctcacgcggttatttaatgggcctaacacgtgttgttcagggttttctaaagagattcctattttcttggatgcacaaattacctattgccacacggccaacaacatgacaacgtcctatacagactatcctgaagtgaaaggttcttggggtaatgataccctgggcttaactttgtctactagcgtacctatcccggtatttagttcttctatctttgatagttatgcaccgtttgcaaaattacaagttgtctatgcgcaccaagatgactttaaagaaccaacaacagaaggccgggtctttgaaagcagcgatcttctcaacgtttctgtacctataggtataaaat

SEQ ID NO: 55 - protein, 162 aa, Chlamydia psittaci
TSREDSLSVAFCQLFAKDKDYLVSKNAANVYAGSVYYQHVSKFDDLTRLFNGPNTCCSGFSKEIPIFLDAQITYCHTANNMTTSYTDYPEVKGSWGNDTLGLTLSTSVPIPVFSSSIFDSYAPFAKLQVVYAHQDDFKEPTTEGRVFESSDLLNVSVPIGIK

SEQ ID NO: 56 - DNA, 2781 bp, Chlamydia psittaci
atgaggccttctttatataagattttaatatcgtcgacgctgacgttaccaatatcttttcacttctcgcaattgcatgcagaagtggctttaactcaagaatctattctcgatgcaaatggagcattcagtccgcaatctacaagcactgcgggaggaacgatttacaacgtcgagagtgatatttctattgtagatgtaggacagacagcggctcttgcttcctcagcttttgttcagactgcagacaacctaactttcaaagggaacaaccatagcttatccataacgaacgcgaatgccggagctaatcctgcgggaattaacgttaacactgccgataagattcttacgctgacagatttttctaagttgagctttaaggaatgcccatcttctctagtgaatactggaaaaggggctatgaaatccggaggagcattaaacttagcgaataatgccagtattctgtttgatcagaactattccgctgagaatggtggagccatctcttgcaaagctttttctctaaccggctcgagcaaagaaatcagcttcaccactaactctactgcgaaaaaaggtggagcgattgctgctacgggaatagctcatctttcggacaaccaaggcacaatcagattttctgggaacactgctgtgaattctgggggagcagtatattcagaagcttctatgacgattgcaggtaacaaccacgttgcttttagcaacaatgctgtttccggttcatctgatggttgcggtggagctatccattgtagcaaaacaggttcagcaccgacccttactataagagataacaaagtcttgatttttgaggaaaatacttcttcagcaaaaggtggagcgatttacaccgataaactcatattgacttctggtgggcctacggcatttatcaataacaaagttacccatgctacacctaagggtggagctattggtattgctgccaatggagaatgtagcttaaccgctgaacatggggatattacttttgataataacctgatggccacacaagacaatgctacaataaaaagaaatgccattaacattgaaggcaatggtaaattcgtcaacttacgtgcagcgtctggaaagacgatttctttctatgatcctatcacagttgaaggtaatgctgctgatcttctcactttgaataaagctgagggtgataaaacgtataatggaagaattattttttcaggagaaaagctcactgaagaacaagctgctgttgcggataacctaaagacaacatttacacagcctatcactttagctgctggtgaacttgtgttacgcagcggtgtggaagtagaagcaaaaacagtcgtgcaaacagcaggatctttgattctgatggatgcaggcacaaagttatccgcaaaaacagaagatgctacactgacgaatctggctattaatccgaataccttagatgggaaaaaattcgccgtagtcgatgccgttgctgctgggaagaatgtgactttatcaggtgctattggcgttattgatcctacagggaagttttatgaaaaccataagctaaatgatacgttagctttaggaggaattcaactttctgggaaaggttcggtgacaacaaccaacgtgcctagtcatgttgttggtgttgctgaaacccactatggttatcaaggaaactggtctgtcagttgggtcaaagataataactctgatcctaaaacacaaacagcaatctttacctggaataaaacaggatatgttccaaatcctgaacgtcgtgctccgctagtactcaatagcctttggggatcctttatagatttacgttctattcaagatgtcttggaacgtagtgttgatagtattcttgagacacgtcgtggtttgtgggtctctggaattgggaacttcttccataaagatcggaatgctgaaaatcgcaaattccgtcatatcagttcgggatatgtgttaggagccacaacaaatacctcgagagaggattctcttagtgtggctttctgtcagttatttgcaaaagataaagactaccttgtaagcaagaacgccgcaaacgtctatgcgggttctgtatattatcagcatgtgagcaagtttgatgatctcacgcggttatttaatgggcctaacacgtgttgttcagggttttctaaagagattcctattttcttggatgcacaaattacctattgccacacggccaacaacatgacaacgtcctatacagactatcctgaagtgaaaggttcttggggtaatgataccctgggcttaactttgtctactagcgtacctatcccggtatttagttcttctatctttgatagttatgcaccgtttgcaaaattacaagttgtctatgcgcaccaagatgactttaaagaaccaacaacagaaggccgggtctttgaaagcagcgatcttctcaacgtttctgtacctataggtataaaatttgagaaactctcctatggagagagaagtgcttatgatcttacactgatgtatatacctgatgtgtaccgtcataatccaagctgtatgacaggattggcgatcaatgacgtttcctggttaaccacagctacgaatcttgctagacaagctttcatagttcgcgcgggtaaccatattgccttaacctctggtgttgagatgttcagtcagtttggtttcgaattacgaagctcttcaagaaattataacgtagatcttggcgctaaggtcgcgttctaa

SEQ ID NO: 57 - protein, 926 aa, Chlamydia psittaci
MRPSLYKILISSTLTLPISFHFSQLHAEVALTQESILDANGAFSPQSTSTAGGTIYNVESDISIVDVGQTAALASSAFVQTADNLTFKGNNHSLSITNANAGANPAGINVNTADKILTLTDFSKLSFKECPSSLVNTGKGAMKSGGALNLANNASILFDQNYSAENGGAISCKAFSLTGSSKEISFTTNSTAKKGGAIAATGIAHLSDNQGTIRFSGNTAVNSGGAVYSEASMTIAGNNHVAFSNNAVSGSSDGCGGAIHCSKTGSAPTLTIRDNKVLIFEENTSSAKGGAIYTDKLILTSGGPTAFINNKVTHATPKGGAIGIAANGECSLTAEHGDITFDNNLMATQDNATIKRNAINIEGNGKFVNLRAASGKTISFYDPITVEGNAADLLTLNKAEGDKTYNGRIIFSGEKLTEEQAAVADNLKTTFTQPITLAAGELVLRSGVEVEAKTVVQTAGSLILMDAGTKLSAKTEDATLTNLAINPNTLDGKKFAVVDAVAAGKNVTLSGAIGVIDPTGKFYENHKLNDTLALGGIQLSGKGSVTTTNVPSHVVGVAETHYGYQGNWSVSWVKDNNSDPKTQTAIFTWNKTGYVPNPERRAPLVLNSLWGSFIDLRSIQDVLERSVDSILETRRGLWVSGIGNFFHKDRNAENRKFRHISSGYVLGATTNTSREDSLSVAFCQLFAKDKDYLVSKNAANVYAGSVYYQHVSKFDDLTRLFNGPNTCCSGFSKEIPIFLDAQITYCHTANNMTTSYTDYPEVKGSWGNDTLGLTLSTSVPIPVFSSSIFDSYAPFAKLQVVYAHQDDFKEPTTEGRVFESSDLLNVSVPIGIKFEKLSYGERSAYDLTLMYIPDVYRHNPSCMTGLAINDVSWLTTATNLARQAFIVRAGNHIALTSGVEMFSQFGFELRSSSRNYNVDLGAKVAF

SEQ ID NO: 58 - DNA, 559 bp, Chlamydia psittaci
tgtgttcattctttagcaggagttgcatttacgttgtttctctgtgagcatatgtttaccaatatgcttgcttcttcttattttaaggaaggcagtggttttgttcagttagtgagcaaatttcatcagattcctggtctgaagatcatagaaattgtttttttagccctaccgtttacttgtcacgctatcctaggtattttctatctttttcaagcgcaaactaattcacgggcttctgacggcagaaaacccgcgttaatctatgcgagaaatcttgcctatacttggcagagaagaactgcttggattttacttttcggtcttatttttcacgtagttcagtttcgttttcttcgttatcctattcatgtagagctgcatgggcaaacatactatgttgtcgatattgacgcttctcggtatgcggcgatagtgcggggtacacaaggattttttactataaatttttcagctcctcaacttgaaacgattcgtttggataaagaggatcttgacggcagcgcagtttctcaattattagacagaaaagcgtatc

SEQ ID NO: 59 - protein, 186 aa, Chlamydia psittaci
CVHSLAGVAFTLFLCEHMFTNMLASSYFKEGSGFVQLVSKFHQIPGLKIIEIVFLALPFTCHAILGIFYLFQAQTNSRASDGRKPALIYARNLAYTWQRRTAWILLFGLIFHVVQFRFLRYPIHVELHGQTYYVVDIDASRYAAIVRGTQGFFTINFSAPQLETIRLDKEDLDGSAVSQLLDRKAY

SEQ ID NO: 60 - DNA, 687 bp, Chlamydia psittaci
atgatgaatgaaaaggaatcatgttctgaggctactcagaggtcatggaagtactacactagctttgttttacgttgtgttcattctttagcaggagttgcatttacgttgtttctctgtgagcatatgtttaccaatatgcttgcttcttcttattttaaggaaggcagtggttttgttcagttagtgagcaaatttcatcagattcctggtctgaagatcatagaaattgtttttttagccctaccgtttacttgtcacgctatcctaggtattttctatctttttcaagcgcaaactaattcacgggcttctgacggcagaaaacccgcgttaatctatgcgagaaatcttgcctatacttggcagagaagaactgcttggattttacttttcggtcttatttttcacgtagttcagtttcgttttcttcgttatcctattcatgtagagctgcatgggcaaacatactatgttgtcgatattgacgcttctcggtatgcggcgatagtgcggggtacacaaggattttttactataaatttttcagctcctcaacttgaaacgattcgtttggataaagaggatcttgacggcagcgcagtttctcaattattagacagaaaagcgtatctcctgactcctaatgttggaccgcttttctttatgttgttcgggattctttag

SEQ ID NO: 61 - protein, 228 aa, Chlamydia psittaci
MMNEKESCSEATQRSWKYYTSFVLRCVHSLAGVAFTLFLCEHMFTNMLASSYFKEGSGFVQLVSKFHQIPGLKIIEIVFLALPFTCHAILGIFYLFQAQTNSRASDGRKPALIYARNLAYTWQRRTAWILLFGLIFHVVQFRFLRYPIHVELHGQTYYVVDIDASRYAAIVRGTQGFFTINFSAPQLETIRLDKEDLDGSAVSQLLDRKAYLLTPNVGPLFFMLFGIL